(19) United States
(12) Patent Application Publication
(10) Pub. No.: US 2023/0372307 A1
(43) Pub. Date: Nov. 23, 2023

(54) COMBINATION THERAPY USING BAX ACTIVATOR AGENT

(71) Applicant: Albert Einstein College of Medicine, Bronx, NY (US)

(72) Inventor: Evripidis Gavathiotis, Roslyn, NY (US)

(21) Appl. No.: 18/044,606

(22) PCT Filed: Sep. 17, 2021
(86) PCT No.: PCT/US2021/050965

§ 371 Date: Mar. 9, 2023

Related U.S. Application Data
(60) Provisional application No. 63/079,720, filed on Sep. 17, 2020.
(60) Provisional application No. 63/109,097, filed on Nov. 3, 2020.

(51) Int. Cl. A61K 31/427; A61K 31/635; A61P 35/00; C12Q 1/6886

(52) U.S. Cl. CPC A61K 31/427; A61K 31/635; A61P 35/00; C12Q 1/6886; C12Q 2600/106; C12Q 2600/158

(57) ABSTRACT

A pharmaceutical combination comprising a B-cell lymphoma 2 associated X protein (BAX) activating compound an anti-apoptotic protein inhibiting compound is provided. The disclosure also provides a method of treating cancer in a subject by administering a B-cell lymphoma 2 associated X protein (BAX) activating compound in combination with an anti-apoptotic protein inhibiting compound, such as a BCL-XL, BCL-2, BFL-1. BCL-w, or MCL-1 inhibiting compound.

### CROSS REFERENCE TO RELATED APPLICATION

This application claims priority of U.S. Provisional Appl. Ser. No. 63/109,097, filed Nov. 3, 2020 and 63/079,720, filed Sep. 17, 2020, both of which are hereby incorporated by reference in their entirety.

### FEDERAL RESEARCH STATEMENT

This invention was made with government support under grant number T32GM007491 awarded by the National Institute of Health, and under grant numbers R01CA178394, F31CA236434, P30CA013330, and 1S10D01630 awarded by the National Institute of Health, NCI. The government has certain rights in the invention.

### BACKGROUND

Deregulated apoptosis is a hallmark of cancer. Cancer cells prevent apoptosis to ensure their survival and growth and becoming resistant to current treatments. The intrinsic or mitochondrial pathway of apoptosis is regulated by the BCL-2 family of proteins that includes the pro-apoptotic or effector proteins (BAX, BAK and BOK), the anti-apoptotic or survival proteins (e.g., BCL-2, BCL-w, BFL-1, BCL-XL, MCL-1), and the pro-apoptotic BH3-only proteins classified either as activators (e.g., BIM, BID) or sensitizers (e.g., BAD, HRK)). Frequently, cancer cells upregulate anti-apoptotic BCL-2 family members to inhibit pro-apoptotic BCL-2 members BAX, BAK, and BH3-only proteins to prevent apoptosis. More resistant cancers also downregulate or inactivate pro-apoptotic BH3-only proteins to suppress apoptosis, making these tumors more insensitive to current treatments.

Pro-apoptotic BAX is an effector of mitochondrial apoptosis induced by most BH3-mimetics and chemotherapeutic agents. Typically, upon a pro-apoptotic stimulus, BH3-only proteins use their BH3-domain helix to trigger BAX activation leading to BAX translocation and oligomerization at the mitochondrial outer membrane (MOM). This causes MOM permeabilization (MOMP) and release of apoptogens such as cytochrome c and Smack/Diablo that activate the caspase cascade of apoptosis. The elucidation of the BAX trigger site where the BH3-domain helix binds to induce BAX activation, enabled the discovery of direct small-molecule BAX activators that engage the trigger site and mimic BH3-only proteins, thus inducing complete conformational activation of BAX and apoptosis.

Direct BAX activation with targeted small molecules can impede the downregulation of activator BH3-only proteins in cancer. However, there remains a need to develop additional and improved direct BAX activators, particularly for tumors refractory to apoptosis.

### SUMMARY

This disclosure provides a pharmaceutical combination, comprising: a B-cell lymphoma 2 associated X protein (BAX) activating compound; and an anti-apoptotic protein inhibiting compound, such as a B-cell lymphoma 2-extra-large protein (BCL-XL) inhibiting compound, a B Cell Lymphoma 2 (BCL-2) inhibiting compound, a B Cell Lymphoma 2 like protein (BCL-w) inhibiting compound, a Myeloid Cell Leukemia 1 (MCL-1) inhibiting compound, a BFL-1 inhibiting compound, or a BCL-B inhibiting compound

In the pharmaceutical combination the BAX activating compound is a compound having a structure of BTSA1 or BTSA1.2, or a pharmaceutically acceptable salt thereof.

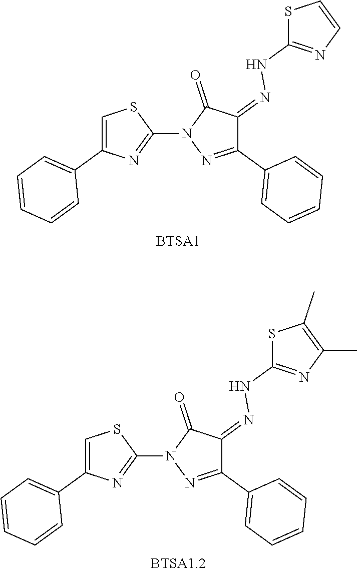 [SMILES: O=C1/C(=N\Nc2nccs2)C(c2ccccc2)=NN1c1nc(-c2ccccc2)cs1 ; Cc1nc(N/N=C2\C(=O)N(c3nc(-c4ccccc4)cs3)N=C2c2ccccc2)sc1C]

The disclosure also provides a method of treating cancer in a subject in need thereof, the method comprising: obtaining a biological sample comprising cancer cells from the subject; detecting a level of BAX:BCL-XL, BAX:BCL-2, BAX:BCL-w, BAX:BFL-1, or BAX: MCL-1 complexes immunoprecipitated from the cancer cells and/or detecting that the cancer cells are anti-apoptotic BCL-XL, BCL-2, BCL-w, BFL-1, or MCL-1 dependent or unprimed to apoptosis; and administering to the subject an anticancer agent comprising a B-cell lymphoma 2 associated X protein (BAX) activating compound and a B-cell lymphoma-extra large protein (BCL-XL) inhibiting compound, or B Cell Lymphoma 2 (BCL-2) inhibiting compound, or Cell Lymphoma 2 like (BCL-w) inhibiting compound or a Myeloid Cell Leukemia 1 (MCL-1) inhibiting compound in an amount effective to treat the cancer.

The disclosure provides a method of treating cancer in a subject in need thereof, the method comprising: obtaining a biological sample from the subject; measuring expression level of at least one gene in the biological sample, wherein the gene comprises MUC13, EPS8L3, IGFBP7, or a combination thereof; and administering to the subject an anticancer agent comprising a B-cell lymphoma 2 associated X protein (BAX) activating compound and an anti-apoptotic protein inhibiting compound. The method can include comparing the expression level of the MUC13, EPS8L3, or IGFBP7 gene, or a combination thereof in the biological sample to a standard expression level for any of these genes and administering the anticancer agent comprising a B-cell lymphoma 2 associated X protein (BAX) activating compound and an anti-apoptotic protein inhibiting compound if the expression level of the gene in the biological sample is higher than the expression level of the gene is higher than the standard expression level. The MUC13, EPS8L3, IGFBP7 markers were identified using BTSA1.2/ Navitoclax combination. Navitoclax is considered a BCL-XL and BCL-2 inhibitor. In certain embodiments the anti-apoptotic inhibiting compound is a B Cell Lymphoma 2 inhibiting compound or preferably, a B-cell lymphoma-extra large protein (BCL-XL) inhibiting compound.

The above described and other features are exemplified by the following figures and detailed description.

### BRIEF DESCRIPTION OF THE DRAWINGS

The following figures are exemplified embodiments.

FIGS. 1A to 1K. Resistance to BAX activation and BCL-XL inhibition is regulated by BCL-XL upregulation and an umprimed state. FIG. 1A: A diverse collection of cancer cell lines (n=46) were treated for 72 hrs with BTSA1.2. Box plot corresponds to the tissue type mean cell viability IC₅₀ (μM), cell lines were categorized as sensitive (IC₅₀<3 μM) or resistant (IC₅₀>3 μM). FIG. 1B, Correlation of sensitivity to BTSA1.2 with BAX and BCL-XL relative protein levels using Pearson- Correlation. Relative protein levels were normalized to b-Actin loading control, p value was calculated using student t-test. FIG. 1C: BAX translocation after 4 hrs. treatment with BTSA1.2 in BxPC-3 cells. FIG. 1D: BAX co-immunoprecipitation (co-IP) after 4 hrs. treatment with BTSA1.2 in BxPC-3 cells. Data are representative of n=3 independent experiments. FIG. 1E: A diverse collection of cancer cells (n=46) treated for 72 hrs. with Navitoclax. Box plot corresponds to the tissue type mean cell viability IC₅₀ (μM), cell lines were categorized as sensitive (IC₅₀<1.5 μM) or resistant (IC₅₀<1.5 μM) . FIG. 1F: Correlation of sensitivity to Navitoclax with BCL-XL and BAX:BCL-XL relative protein levels using Pearson-Correlation. Relative protein levels were normalized to b-Actin loading control, p value was calculated using student t-test. FIG. 1G: Heatmap representation of % mitochondria depolarization of 20 cancer cell lines classified on different apoptotic blocks based on the BH3 profiling approach. FIGS. 1H to 1I: BH3 profiling predicts apoptotic blocks correlated with resistance to (H) BTSA1.2 and (I) Navitoclax. FIG. 1J: Venn diagram comparing cell lines resistant to BTSA1.2 and Navitoclax as single agents. FIG. 1K: Diagram illustrating the therapeutic strategy of combinatorial treatment with BAX activator (BTSA1.2) and BCLXL inhibitor (Navitoclax) to enhance apoptotic cell death. Data in FIGS. 1G and 1J are mean of three replicates from n=2 independent experiments.

FIGS. 1L-1Q. BTSA1.2, analog of BTSA1, with improved binding to BAX, cellular activity and on target engagement activity. FIGS. 1L-1O: IC50 curves of lymphoma cell lines upon treatment with BTSA1 or BTSA1.2. FIG. 1P: Cellular thermal shift assay (CETSA) of BAX melting curves in BxPC-3 cells treated with vehicle (DMSO) or 40 μM BTSA1.2 for 15 minutes. Blot is representative of three independent experiments. FIG. 1Q: The data from FIG. 1P was quantified by fluorescence intensity using LiCor Odyssey Clx and normalized to generate melting curves. Data are mean±SD from n=3 experiments.

FIGS. 2A to 2H. BTSA1.2 and Navitoclax synergize to inhibit cell viability and induce apoptosis in resistant tumor cell lines. FIG. 2A: Navitoclax and BTSA1.2 (1.25 μM or 5 μM) screening. FIG. 2B: Bar graph plot of the cell viability IC₅₀ (μM) fold change of cancer cell line panels (n=46) treated for 72 hrs. with Navitoclax in combination with a constant sensitizing concentration of BTSA1.2 (loss of cell viability <20%). Red bar graphs correspond to IC₅₀ fold change >5×; green bar graphs correspond to IC₅₀ fold change 2-4×; and gray bar graphs correspond to IC₅₀ fold change <2×. Cells were predicted to be sensitive (IC₅₀ fold change >5×), have intermediate sensitivity (IC₅₀ fold change 2-4×) or resistant (IC₅₀ fold change <2×) to the combination. FIG. 2C: Mutation status of TP53 and RAS in cancer cell lines classified as sensitive or resistant to the combination. FIG. 2D: Dose-response curves of Navitoclax in the presence of various doses of BTSA1.2 in a panel of resistant cancer cell lines to single agents (Leukemia=U937, Colon=SW480, Pancreatic=BxPC-3, NSCLC=Calu-6), n=3. FIG. 2E: Bliss synergy score heat map from combinatorial treatment of BTSA1.2 and Navitoclax in different cancer tissue types in b, n=3. FIG. 2F: Caspase 3/7 activity assay in diverse cancer cell lines treated with BTSA1.2 and Navitoclax alone or in combination measured at 8 hrs., n=3. FIG. 2G: Cell viability at 24 hrs in WT and CRISPR/Cas9 BAX KO Calu-6 cell lines treated with Navitoclax alone in the presence of a fixed sensitizing concentration of BTSA1.2 (loss of viability <10%). Comparison of BAX and BAK protein expression levels in indicated cell lines, n=3. FIG. 2H: Caspase 3/7 activity in WT and CRISPR/Cas9 BAX KO Calu-6 cell lines after 8 hrs. treatment with Navitoclax alone and in combination with a fixed sensitizing concentration of BTSA1.2 (loss of viability <10%), n=3. Statistics were obtained using two-way ANOVA: *, p<0.05; **, p<0.01; ***, p<0.001; ****, p<0.0001.

FIG. 2I-2J. FIG. 2L Cell viability at 72 hrs in WT and CRISPR/Cas9 BAX KO Calu-6 cells lines treated with various doses of staurosporine. FIG. 2J: Caspase 3/7 activity in WT and CRISPR/Cas9 BAX CO Calu-6 cell lines at 24 hrs treatment with various doses of staurosporine. Data are ±SD of four technical replicates from n=3 independent experiments.

FIGS. 3A to 3J. BAX interaction with BCL-XL dictates sensitivity to BTSA1.2 and Navitoclax combination. FIG. 3A: BH3 profiling predicts apoptotic blocks correlated with BTSA1.2 and Navitoclax combination sensitivity. FIGS. 3B-3C Western blot analysis of BAX Co-IP in a panel of (FIG. 3B) NSCLC and (FIG. 3C) colorectal cells. FIG. 3D: Quantification of co-immunoprecipitated BAX with BCL-XL in solid tumor cell lines panel grouped between sensitivity towards the BTSA1.2 and Navitoclax combination (FIG. 3B-C). FIGS. 3E-3F: Western blot analysis of BAX IP in (FIG. 3E) NSCLC cancer cell line Calu-6 and (FIG. 3F) colorectal cell line SW480 after 4 hrs. treatment with BTSA1.2 and Navitoclax. FIGS. 3G-3H: Detection of cleaved Caspase-3 apoptotic marker by western blot analysis in (3G) NSCLC cancer cell line Calu6 and (FIG. 3H) colorectal cell line SW480 after 4 hrs. treatment with BTSA1.2 and Navitoclax. FIG. 3I: Schematic of sensitive cells to the BTSA1.2 and Navitoclax combination. Data are representative of three independent experiments. FIG. 3J: Apoptotic priming with activator BIM BH3 peptide increased upon the combination treatment in sensitive cell lines but not on resistant cells.

FIGS. 4A to 4G. Combination of BTSA1.2 and Navitoclax is well tolerated and does not enhance Navitoclax driven toxicity in the hematopoietic system. FIG. 4A: Schematic of BTSA1.2 and Navitoclax combination toxicity study. FIG. 4B: Body weight measurements of CD1-IGS mice 0, 3, 7, 11 and 14 days after the first treatment with vehicle, 100 mg/kg Navitoclax, 200 mg/kg BTSA1.2 or the combination. FIGS. 4C-4F: Counts of peripheral (C) red blood cells, (D) white blood cells, (E) lymphocytes, and (F) platelets in CD1-IGS mice treated with vehicle, 100 mg/kg Navitoclax, 200 mg/kg BTSA1.2 or the combination after treatment 1 and 7 days after treatment. Normal blood counts range for CD-IGS male mice are indicated in gray. Data in (FIGS. 4B-4F) represent mean±SD (Vehicle, BTSA1.2 and Navitoclax n=5, Combination n=6). Scale bars, 100 μm. Statistics were obtained using student t-test: *, p<0.05; **, p<0.01; ***, p<0.001; ****, p<0.0001. FIG. 4G: Representative tissue sections spleen, bone marrow, heart, liver, brain, lungs and kidney using Hematoxylin and Eosin (H&E) staining from mice after treatment of vehicle, 100 mg/kg Navitoclax, 200 mg/kg BTSA1.2 or the combination.

FIGS SA to 5I. Combination therapy of BTSA1.2 and Navitoclax shows potent efficacy in resistant colorectal tumor xenografts. FIG. SA: Schematic of SW480 xenograft efficacy study. FIG. 5B: Body weight measurements of Nu/Nu mice at 0, 7, and last day of treatment with vehicle, 100 mg/kg Navitoclax, 200 mg/kg BTSA1.2 or the combination. FIG. SC: Tumor volume curves of vehicle, Navitoclax, BTSA1.2 or the combination cohorts. FIG. SD: Tumor weight after completing study. Data in (B-D) represent mean ±SD (Vehicle, BTSA1.2 and Navitoclax n=5, Combination n=6). FIG. 5E: Schematic of SW480 pharmacodynamic xenograft study. FIG. 5F: Example of kinetic curve of mitochondria potential in tumors treated with Vehicle or combination upon stimuli of BH3-BIM peptide, Puma2A, CCCP or Alamethicin. FIG. 5G: Dynamic BH3 profiling of tumors from mice treated with vehicle or Navitoclax and BTSA1.2 combination. Bar graph represent % of mitochondria depolarization of tumor cells detected by JC-1 upon treatment BH3-BIM derived peptide or DMSO (Vehicle n=2; Combination n=3). FIGs. SH-SI, Detection of cleaved Caspase-3 and cleaved PARP apoptotic markers by Western Blot analysis from SW480 tumors, n=3. Relative protein levels were normalized to b-Actin loading control. Statistics were obtained using one-way Anova: *, p<0.05; **, p<0.01; ***, p<0.001; ****, p<0.0001.

FIGS. 6A to 6L. Predictive markers identify sensitive tumors to the combination therapy of BTSA1.2 and Navitoclax. FIG. 6A: Schematic of tumors characterization by BH3-Profiling and BAX co-IP to predict clinical sensitivity. FIG. 6B: BH3-Profile of colorectal PDXs. Heatmap represent % of mitochondria depolarization of isolated tumor cells detected by JC-1 upon treatment BH3-derived peptides, n=3. FIG. 6C: Quantification of co-immunoprecipitated BAX with BCL-XL in colorectal PDX. FIG. 6D: Cell viability of COLO-1 PDX isolated cells after 24 hrs. treatment with 1.25 μM Navitoclax, 10 μM BTSA1.2 or combination, n=3. FIG. 6E: Schematic of COLO-1 PDX efficacy study. FIG. 6F: Body weight measurements of NOD SCID mice at 0, 6, and last day of treatment with vehicle, 50 mg/kg Navitoclax, 200 mg/kg BTSA1.2 or the combination. FIG. 6G: Tumor volume curves of vehicle, Navitoclax, BTSA1.2 or the combination cohorts. Data in FIGS. 6F-6G represents individual measurements (vehicle n=9, BTSA1.2, Navitoclax, and combination n=12). FIG. 6H: Survival of COLO-1 PDX after 18 days of treatment with vehicle, 50 mg/kg Navitoclax, 200 mg/kg BTSA1.2 or the combination, n=8. FIG. 6I: Dynamic BH3-profiling of COLO-1 tumors from mice treated with vehicle or Navitoclax and BTSA1.2 combination. Bar graph represent % of mitochondria depolarization of tumor cells detected by JC-1 upon treatment BH3-BIM, BH3-BID or Puma2A derived peptide, n=2. FIG. 6J: Schematic of COLO-2 PDX efficacy study. FIG. 6K: Tumor volume curves of vehicle, Navitoclax, BTSA1.2 or the combination cohorts. FIG. 6L: Tumors of mice treated with BTSA1.2 or Navitoclax had a significant increase of BCL-XL protein levels while MCL-1 levels remained constant. Data represents individual measurements (vehicle n=5, BTSA1.2, Navitoclax, and combination n=8). Statistics were obtained using one-way Anova: *, p<0.05; **, p<0.01; ***, p<0.001; ****, p<0.0001

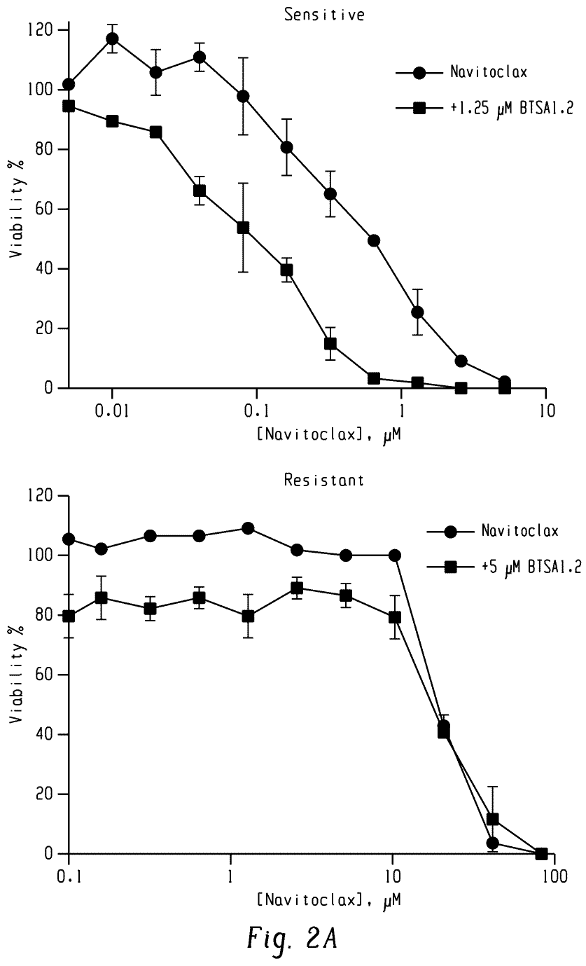

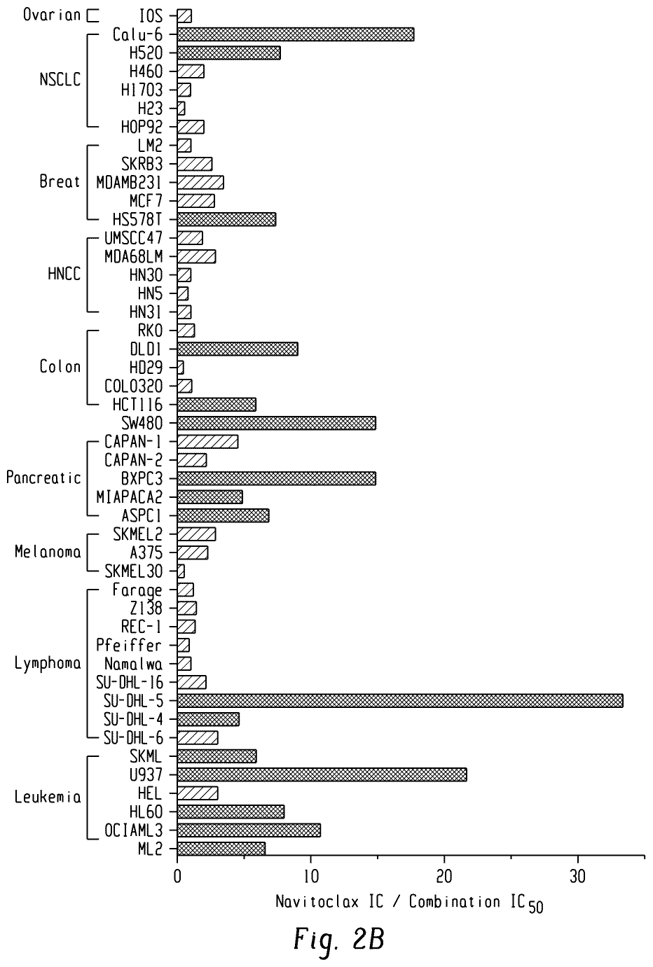

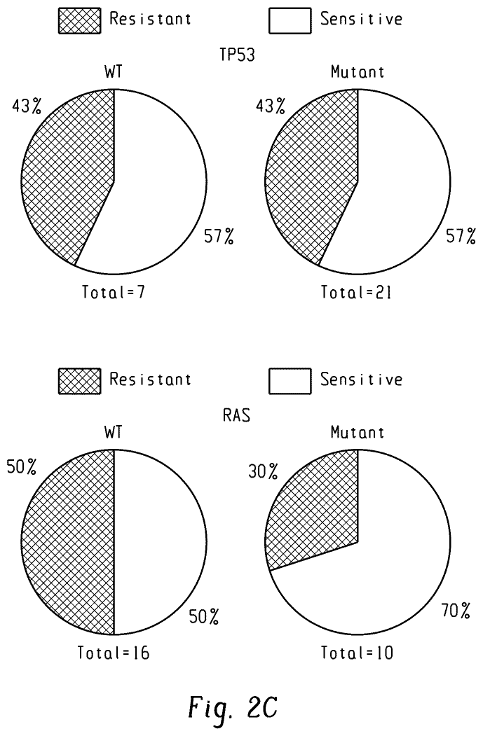

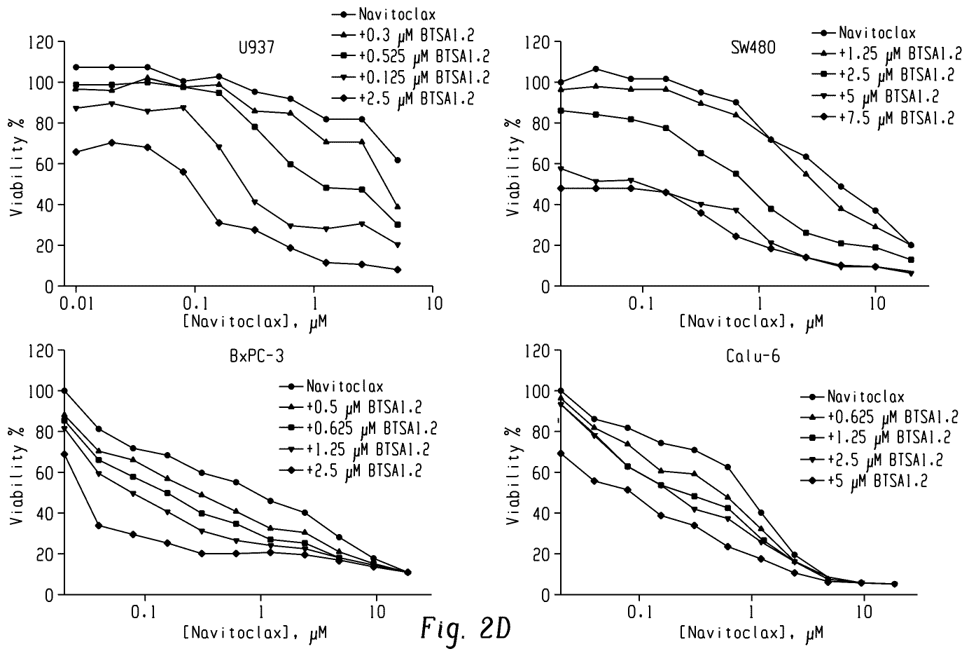

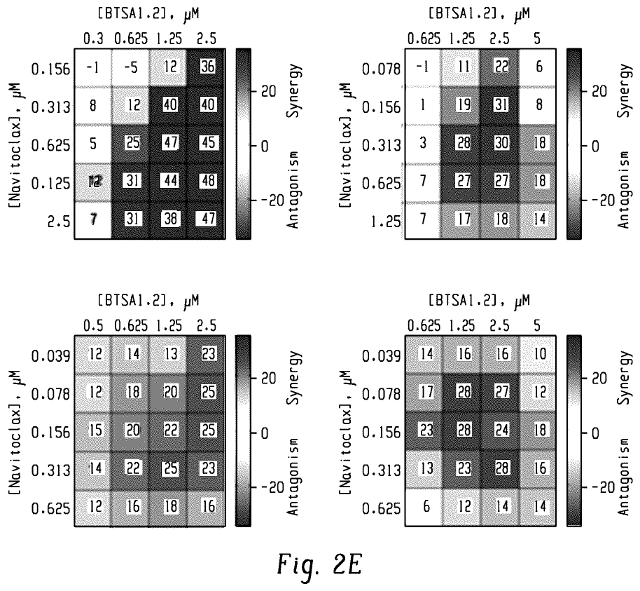

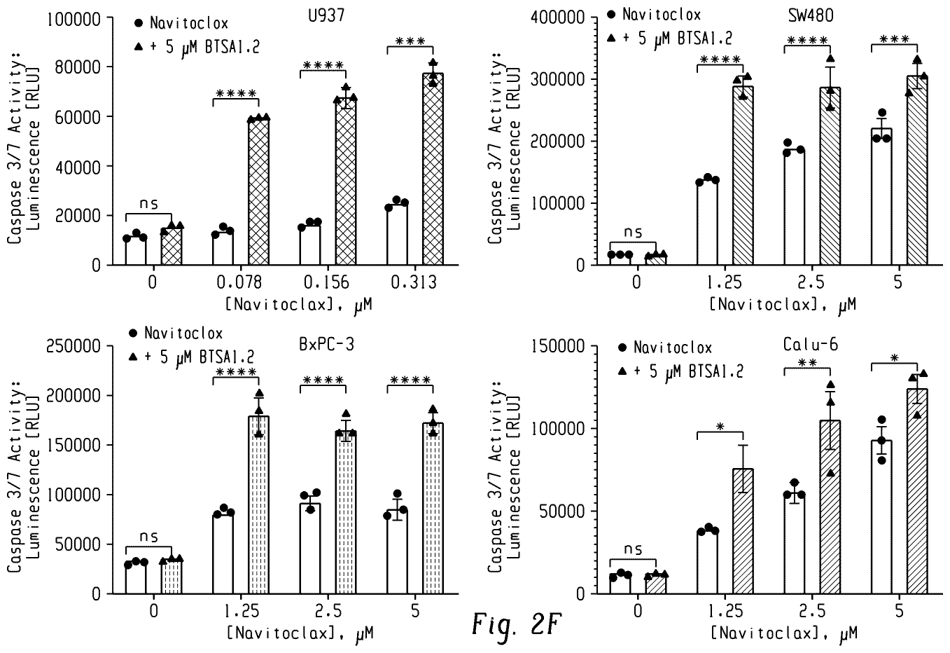

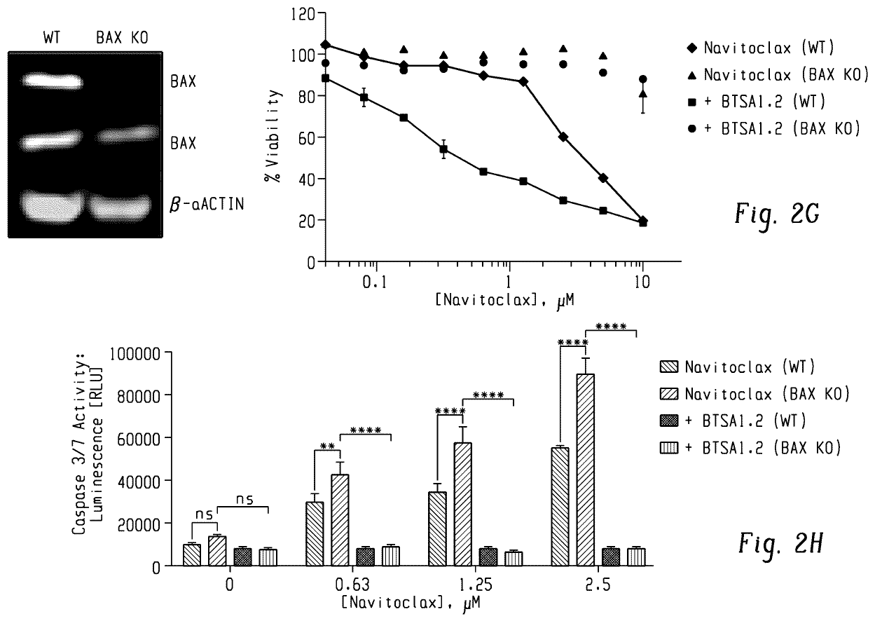

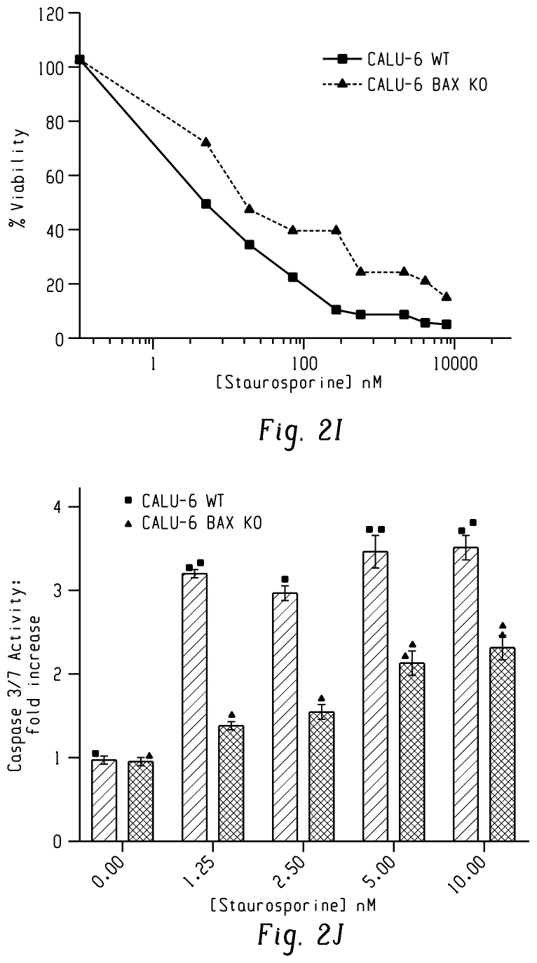

FIGS. 7A to 7E. Bioinformatic analysis predicts markers of sensitivity and resistance to the BTSA1.2 and Navitoclax combination. FIG. 7A: Volcano plot showing the expression change and significance level of genes between sensitive and resistant cell lines group defined by their IC₅₀ change from Navitoclax alone to BTSA1.2 and Navitoclax combined (corresponding to FIG. 2B). Top 250 predicted markers of sensitivity (red) and resistance (gray) are highlighted. FIG. 7B: Validation of top hits associated with sensitivity and resistance to the combination by RT-qPCR in cell lines categorized as sensitive or resistant to the combination. Relative gene expression was normalized using RPL27. FIG. 7C: Correlation of BCL2L1 (corresponds to BCL-XL protein) relative gene expression levels and MUC13 gene expression levels in cell lines categorized as sensitive or resistant to the combination (corresponding to FIG. 2B) using Pearson-Correlation. FIG. 7D: Correlation of MUC13 expression with sensitivity to the combination (corresponding to FIG. 2B). FIG. 7E: MUC13 cancer patient's expression data using TCGA and other non-redundant data from cbioportal.org. Statistics were obtained using student t-test: *, p<0.05; **, p<0.01; ***, p<0.001; ****, p<0.0001.

FIGS. 8A to 8B. BTSA1.2, an improved analog of BTSA1, has activity in a diverse collection of human cancer cell lines. FIG. 8A: Structures of BTSA1 and BTSA1.2. FIG. 8B: Competitive fluorescence polarization assay of BTSA1 and BTSA1.2 competing BIM BH3 binding to BAX. Data are representative of n=2 independent experiments.

FIGS. 9A-9B. BTSA1.2 activity in a diverse collection of human cancer cell lines. FIG. 9A: Cell viability curves of diverse cell lines upon treatment with BTSA1.2 for 72 hrs. FIG. 9B: Bar graph plot of the cell viability IC₅₀ (μM) arranged by sensitivity, red IC_{═}21 3 μM; orange 3<IC₅₀10 μM; yellow IC₅₀>10 μM. Data are mean±SD of three technical replicates from n=2 independent experiments.

FIGS. 10A to 10D. BCL-2 family protein expression levels in solid tumor and hematological cancer cell lines and correlation analysis of BTSA1.2 activity. FIG. 10A) Protein expression levels of key BCL-2 family members detected by Licor. β-Actin was used as loading control. FIGS. 10B-10D: Correlation of sensitivity to BTSA1.2 with (FIG. 10B) MCL-1, BCL-2, (FIG. 10C) BIM, BAK and (FIG. 10D) BAX:BCL-XL relative protein levels using Pearson-Correlation. Relative protein levels were first normalized to β-Actin loading control, p value was calculated using student t-test. Data are representative of n=2 independent experiments.

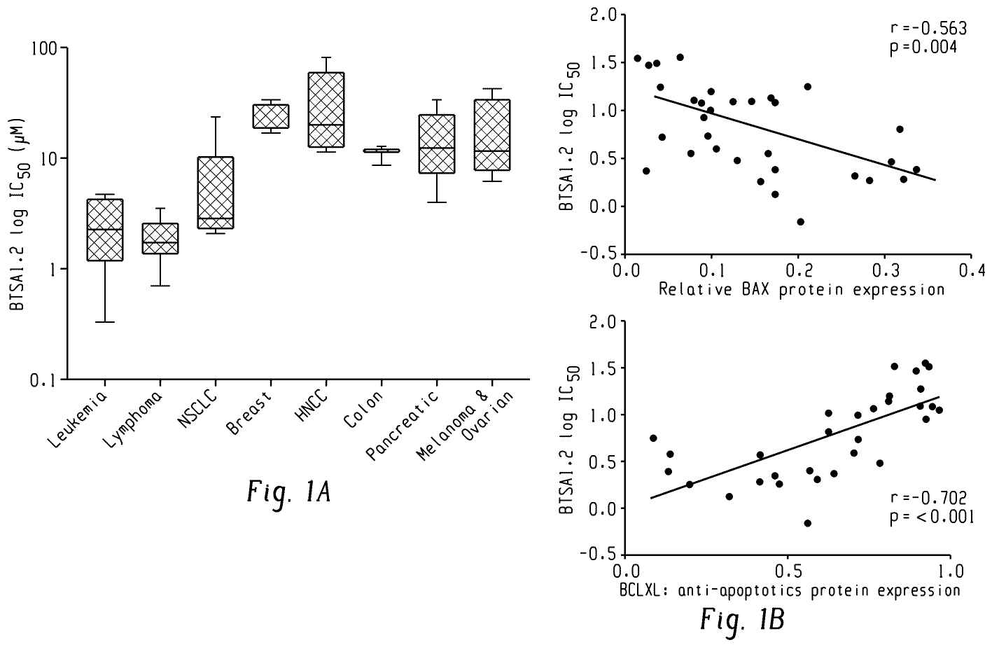

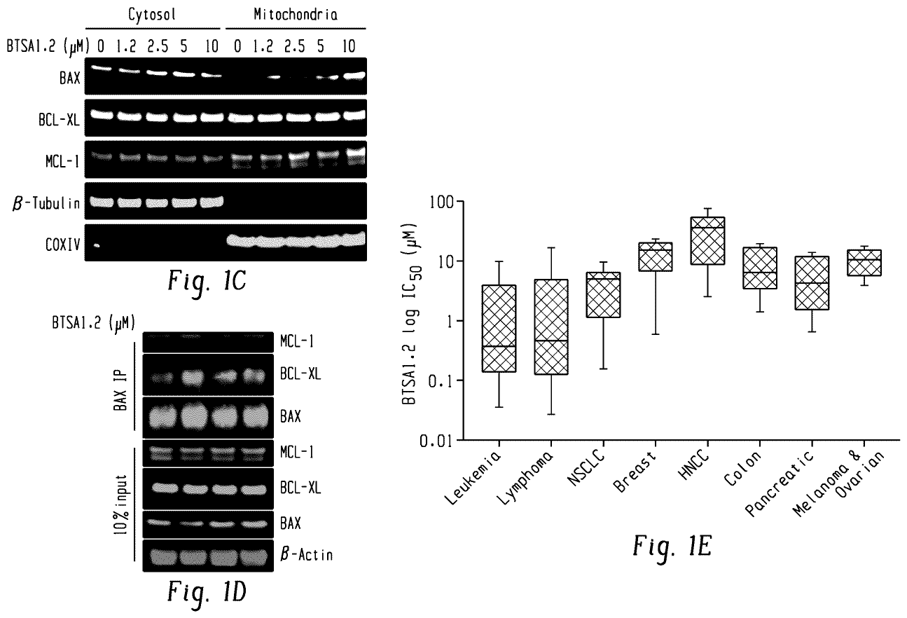

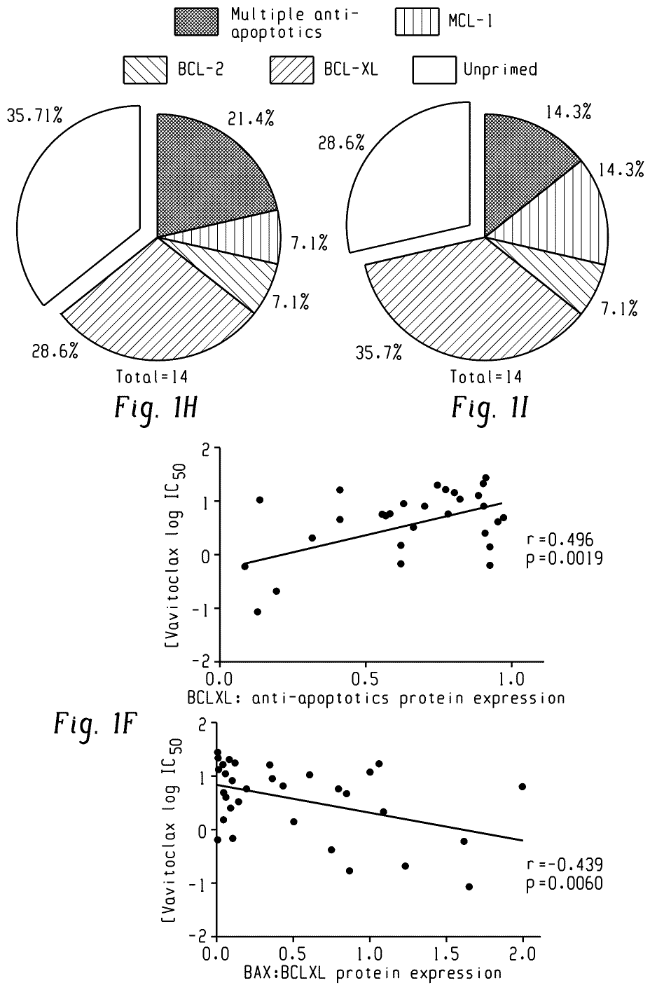

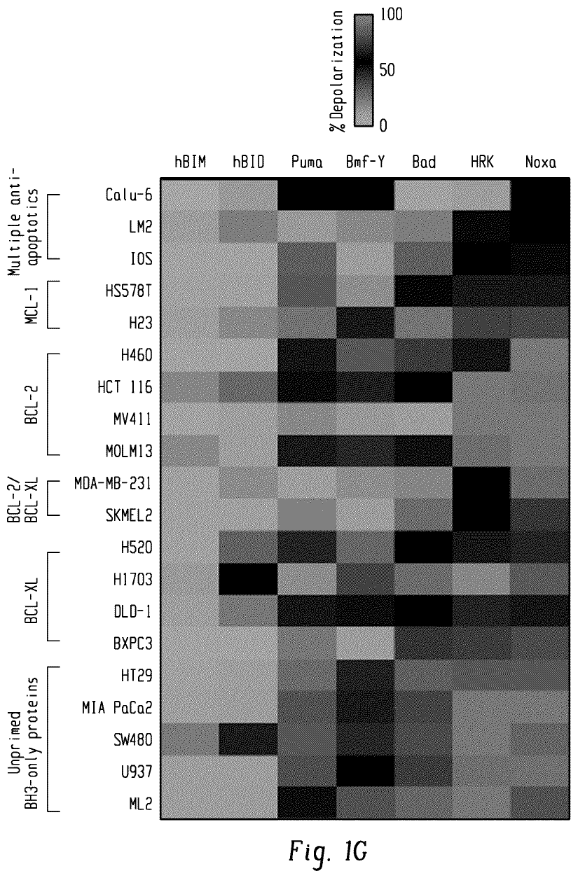

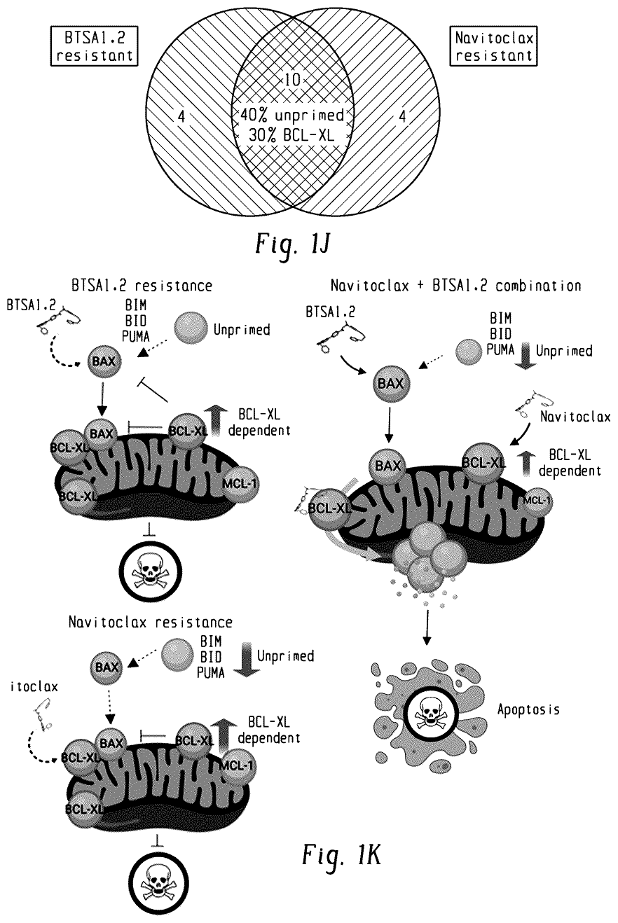

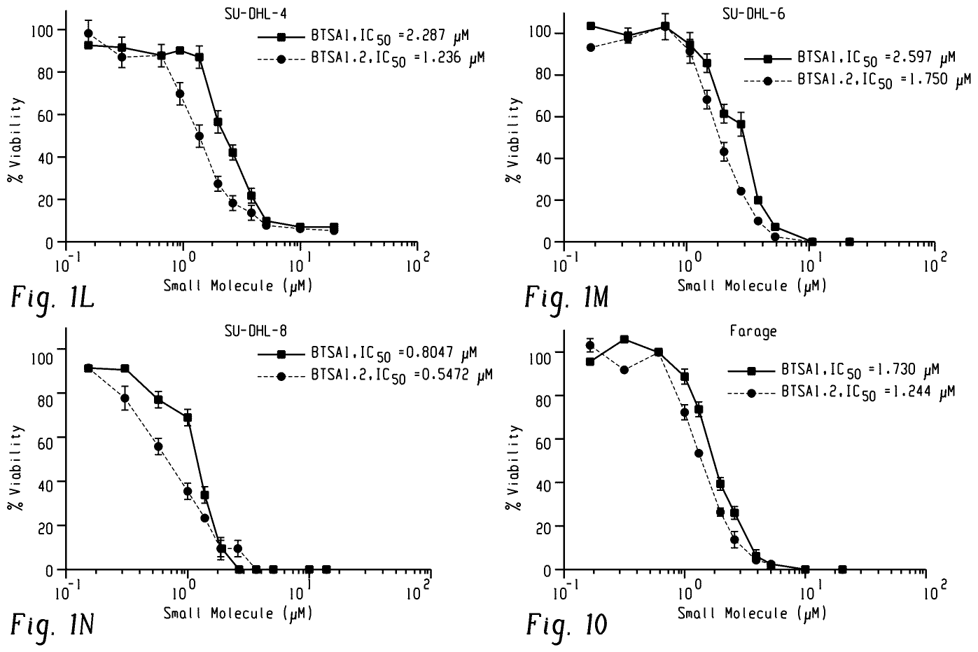

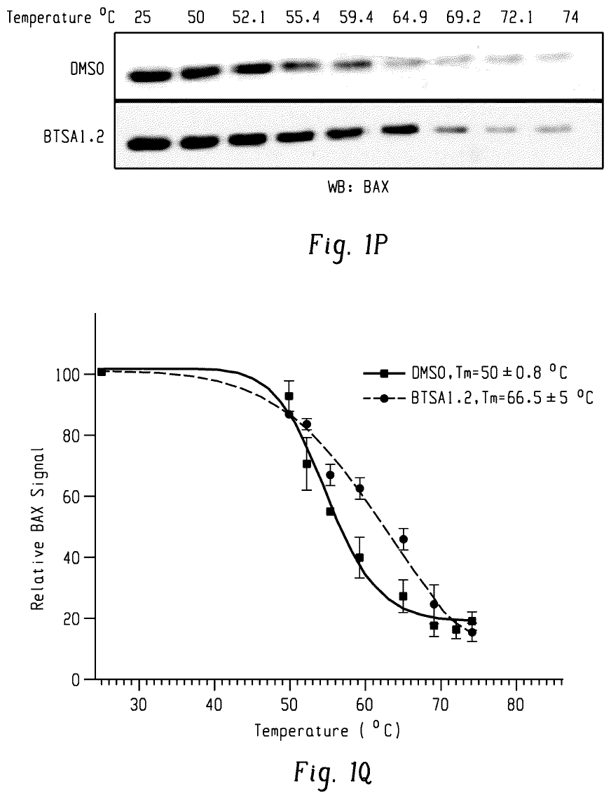

FIGS. 11A to 11G. BCL-XL regulates BAX activation by BTSA1.2 resistance. FIG. 11A: Quantification of translocated BAX upon 4 hrs. treatment with BTSA1.2 in BxPC-3 cell line (related to FIG. 11C). FIG. 11B: BAX translocation upon 4 hrs treatment with BTSA1.2 in SW40 cell line. FIG. 1C: BAX translocation upon 18 hrs treatment with BTSA1.2 in BxPC-3 cell line. FIG. 11D: Quantification of co-immunoprecipitated BAX with anti-apoptotic BCL-XL and MCL-1 upon 4 hrs. treatment with BTSA1.2 in BxPC-3 cell line (related to FIG. 1D). FIG. 11E: Mitochondrial and cytosolic BAX co-IP upon 4 hrs. treatment with 10 μM BTSA1.2 in BxPC-3 cell line. FIG. 11E: Mitochonrdial and cytosolic BAX co-IP upon 4 hrs treatment with 10 μM BTSA1.2 in BxPC-3 cell line. Data are representative of at least n=3 independent experiments. Western blot analysus of BAX Co-IP in a panel of NSCLC (FIG. 11F) and colorectal cells (FIG. 11G). Data are representative of n=3 independent experiments.

FIGS. 12A to 12C. Navitoclax activity in a diverse collection of human cancer cell lines and correlation analysis of Navitoclax activity. FIG. 12A: Cell viability curves of cell lines upon treatment with Navitoclax for 72 hrs. FIG. 12B: Bar graph plot of the cell viability IC₅₀ (μM) arranged by sensitivity, red IC₅₀<1.5 μM; orange 1.5<IC₅₀<10 μM; yellow IC₅₀>10 μM. Correlation of sensitivity to Navitoclax with MCL-1, BCL-2, BIM, BAK and BAX relative protein levels using Pearson-Correlation. FIG. 12C: Relative protein levels were first normalized to β-Actin loading control, p value was calculated using student t-test. Data are ±SD of three technical replicates from at least n=2 independent experiments.

FIGS. 13A to 13D. BH3-Profiling of solid tumor and hematological cancer cell lines. FIG. 13A: % Mitochondria depolarization upon treatment with BH3-only derived peptides. FIG. 13B: BH3 profiling predicts apoptotic blocks correlated with BTSA1.2 sensitivity. FIG. 13C: BH3-profiling predicts apoptotic blocks correlated with Navitoclax sensitivity. FIG. 13D: BH3-profiling predicts apoptotic blocks correlated with BTSA1.2 and Navitoclax resistance.

FIGS. 14A to 14C. BTSA1.2 and Navitoclax combination to inhibit cell viability in resistant tumor cell lines. FIG. 14A Hematological cell lines; FIG. 14B, NSCLC, colorectal, melanoma, and ovarian cell lines; and FIG. 14C, pancreatic, breast, and HNCC cell lines. Viability curves of cell lines treated with Navitoclax in combination with a constant sensitizing concentration (induce <20% viability loss) of BTSA1.2. Data are ±SD of three technical replicates from at least n=2 independent experiments.

FIGS. 15A to 15B. BTSA1.2 and Navitoclax synergize to inhibit cell viability in resistant tumor cell lines. FIG. 15A: Dose-response curves of Navitoclax in the presence of various doses of BTSA1.2 in a panel of resistant cancer cell lines to single agents from various tissue types (Colon=DLD1, Pancreatic=Mia PaCa-2, Lymphoma=SU-DHL-5). Effects on cell viability were measured by CellTiter-Glo after 72 hrs. of treatment, n=3. FIG. 15B: Bliss synergy score heat map from combinatorial treatment of BTSA1.2 and Navitoclax in different cancer tissue types. Data are ±SD of three technical replicates from at least n=3 independent experiments.

FIGS. 16A to 16G. Pharmacokinetics and maximum tolerated dose analysis of BTSA1.2. FIG. 16A: Concentrations (ng/mL) of BTSA1.2 in mice plasma after perioral (p.o.) administration of BTSA1.2 at a dose of 3 mg/kg. FIG. 16B: Concentrations (ng/mL) of BTSA1.2 in mice plasma after intravenous (i.v.) administration of BTSA1.2 1 mg/kg at a dose of 1 mg/kg. FIG. 16C: BTSA1.2 is well-tolerated in vivo: Schematic of MTD and toxicity study of BTSA1.2. CD-IGS female and male mice were treated daily with increasing concentration of BTSA1.2 orally administered for 5 days. Body weights and blood counts were measured at indicated days. Mice were sacrificed 14 days after the first treatment and organs where collected for pathology analysis, n=6. FIG. 16D: Body weight measurements of CD1-IGS mice after treatment with vehicle or BTSA1.2. FIG. 16E: Representative tissue sections spleen, heart, liver, lungs and kidney using Hematoxylin and Eosin (H&E) staining from mice after treatment of vehicle or BTSA1.2. Scale bars, 100 μm. FIG. 16F-16G: counts of peripheral white blood cells (FIG. 16F) and neutrophils (FIG. 16G) in CD1-IGS mice treated with vehicle, 200 mg/kg BTSA1, or 200 mg/kg BTSA1.2, at 0 and 2 days after treatment. Compounds were administered orally. Normal blood counts range for CD-IGS male mice are indicated in gray. Data are ±SD from n=3 mice. Statisitc were obtained using student t-test: *, p<0.05; **, p<0.01; ***, p<0.001; ****, p<0.0001.

FIGS. 17A to 17E. Combinatorial therapy of BTSA1.2 and Navitoclax is well tolerated, does not enhance Navitoclax driven toxicity in the hematopoietic system and primes tumors in vivo to apoptosis. FIGS. 17A-17C: Combinatorial therapy of BTSA1.2 and Navitoclax is well tolerated in vivo: Counts of peripheral (17A) red blood cells, (17B) platelets, and (17C) white blood cells in CD1-IGS mice treated with vehicle, 100 mg/kg Navitoclax, 200 mg/kg BTSA1.2 or the combination 0, 1, 2, 7 and 12 days after treatment. Data in FIGS. 17A-17C represents mean±SD (Vehicle, BTSA1.2 and Navitoclax n=5, Combination n=6). Statistics for these panels were obtained using one-way ANOVA: *, p<0.05; **, p<0.01; ***, p<0.001; ****, p<0.0001. FIGS. 17D-17E: Dynamic BH3 profiling of tumors from mice treated with vehicle or Navitoclax and BTSA1.2 combination. FIG. 17D: Example of kinetic curve of mitochondria potential in tumors treated with vehicle or combination upon stimuli of BH3-BID peptide, Puma2A, CCCP or Alamethicin. Data represent mean±SD of three replicates from n=2 independent experiments. FIG. 17E: Bar graph represent % of mitochondria depolarization of tumor cells detected by JC-1 upon treatment BH3-BID derived peptide or DMSO, Vehicle n=2; Combination n=3. Statistics for this panel were obtained using one-way Anova: *, p<0.05; **, p<0.01; ***, p<0.001; ****, p<0.0001.

FIGS. 18A to 18F. Predictive markers for Navitoclax and BTSA1.2 combination sensitivity. FIG. 18A: BH3-Profile of two colorectal PDXs. Bar graph represent % of mitochondria depolarization of isolated tumor cells detected by JC-1 upon treatment BH3-derived peptides. Data represent mean±SD of three replicates from n=3 mice. FIG. 18B: Western blot analysis of BAX Co-IP in colorectal PDX tumors. FIG. 18C: Cell viability of COLO-1 and COLO-2 PDX isolated cells after 24 hrs. treatment with Navitoclax or BTSA1.2, n=3. FIG. 18D: Cell viability of COLO-2 PDX isolated cells after 24 hrs. treatment with 10 μM Navitoclax, 10 μM BTSA1.2 or combination, n=3. FIG. 18E: Heatmap showing top 150 (selected by adjusted p-value) differentially expressed genes comparing resistant and sensitive cell lines based on IC50 change from Navitoclax alone to BTSA1.2 and Navitoclax combined. FIG. 18F: Validation of top hits associated with sensitivity and resistance to the combination by RT-qPCR in cell lines categorized as sensitive or resistant to the combination (corresponding to FIG. 2A). Relative gene expression was normalized using RPL27.

FIGS. 19A and 19B. Cell viability as a function of BAX activator (BTSA1) and BCL-2 inhibitor (Venetoclax) combinations in resistant AML cell lines. FIG. 19A shows synergism of the BTSA1 and Venetoclax combination in THP-1 cells. FIG. 19 B shows synergism of the BTSA1 and Venetoclax combination in OCI-AML3 cells.

FIG. 20. Single agent and combination with venetoclax treatment of 10 primary patient AML samples in PDX. In FIG. 20 Engraftment Percent, measured as the percent change in number of hCD45+ cells, is plotted as a function of time (weeks) for 10 samples of AML tumor cells established as patient derived xenografts (PDX). PDX mice were treated daily for 3 weeks with vehicle only, ABT-199 (Venetoclax) only, BTSA1 only, or Venetoclax in combination with BTSA1. While both Venetoclax and the BTSA1/Venetoclax combination decreased engraftment percent the effects continued post treatment only in animals administered the BTSA1/Venetoclax combination.

FIGS. 21A and 21B. BTSA1 and Venetoclax combination accelerates induction and potency of apoptosis. FIG. 21A shows increased apoptosis as measured by the caspase 3/7 activation assay for BTSA1, Venetoclax, and the BTSA1/Venetoclax combination. FIG. 21B Western blot shows increased BAX activation with the BTSA1/Venetoclax combination.

FIG. 22 shows the complete blood count (CBC) and the percent of several blood cell types in mice treated with vehicle, venetoclax, BAX activator (BTSA1), and the venetoclax/BTSA1 combination.

FIGS. 23A and 23B. Viability of Leukemia Cells (OCI-AML3) treated with Venetoclax only or a combination of BTSA1.2 and Venetoclax. FIG. 23A: Venetoclax or Venetoclax+1.25 μM BTSA1.2, FIG. 23B: Venetoclax or Venetoclax+2.5 μM BTSA1.2

FIGS. 24A and 24B. Viability of HL60 and ML2 Leukemia Cells treated with Navitoclax only or Navitoclax+1.2504 BTSA1.2. FIG. 24A: HL60 cells, FIG. 24B: ML2 cells.

FIGS. 25A and 25B. Viability of SU-DHL-4 and SU-DHL-5 Lymphoma cells treated with Navitoclax only or a combination of Navitoclax+0.500 μM BTSA1.2. FIG. 25A SU-DHL-4 cells, FIG. 25B: SU-DHL-5 cells.

FIGS. 26A-26E. BTSA1.2 and BCL-XL selective inhibitor A1331852 synergize to inhibit cell viability in sensitive tumor cell lines to the Navitoclax/BTSA1.2 combination.

FIGS. 26A and 26B: Dose-response curves of the BCL-XL selective inhibitor A1331852 or the BCL-2 selective inhibitor Venetoclax in the presence of various doses of BTSA1.2 in a sensitive (SW480) or resistant (COLO-320) cancer cell line to the Navitoclax/BTSA1.2 combination. Effects on cell viability were measured by CellTiter-Glo after 72 hrs of treatment. Bliss synergy score heat map from combinatorial treatment. Data are mean±SD of three technical replicates from n=3 independent experiments. FIGS. 26C and 26D: Dose-response curves of the BCL-XL selective inhibitor A1331852 or the BCL-2 selective inhibitor Venetoclax in the presence of various doses of BTSA1.2 in OCI-AML3 or U937 hematologic cell line sensitive to the Navitoclax/BTSA1.2 combination. Effects on cell viability were measured by CellTiter-Glo after 72 hrs of treatment. Bliss synergy score heat map from combinatorial treatment. Data are mean±SD of three technical replicates from n=3 independent experiments. FIG. 26E: BCL-2 family protein levels compared with sensitivity to the BTSA1.2 and Navitoclax combination: Protein expression levels correlation with Combination. Correlation of sensitivity to BTSA1.2 and Navitoclax combination with MCL-1, BCL-XL BCL-2, BIM, BAK and BAX relative protein levels using Pearson-Correlation. Relative protein levels were first normalized to b-Actin loading control, p value was calculated using student t-test. Data are representative of at least n=2 independent experiments

### DETAILED DESCRIPTION

Disclosed herein are pharmaceutical combinations of a B-cell lymphoma 2 associated X protein (BAX) activating compound and a B-cell lymphoma-extra large protein (BCL-XL) inhibiting compound, a B Cell Lymphoma 2 (BCL-2) inhibiting compound, Myeloid Cell Leukemia 1 (MCL-1) inhibiting compound, a BCL-B inhibiting compound, or a BFL-1 inhibiting compound. (BFL-1 is a BCL-1 related protein first identified in fetal liver.) Also disclosed herein are methods of treating cancer in a subject comprising administering to the subject a BAX activating compound in combination with an anti-apoptotic protein inhibiting compound, such as a BCL-XL inhibiting compound, a B Cell Lymphoma 2 (BCL-2) inhibiting compound, or a Myeloid Cell Leukemia 1 (MCL-1) inhibiting compound, in an amount effective to treat the cancer in the subject. When an anti-apoptotic protein inhibiting compound, such as a BCL-XL, BCL-2, or MCL-1 inhibiting compound is administered together with a BAX-activating compound, there is increased death of cancer cells as compared to the BAX-activating compound or the anti-apoptotic protein inhibiting compound, such as a BCL-XL, BCL-2, or MCL-1 inhibiting compound, alone. The present disclosure also pertains to methods of treating a patient by first determining the sensitivity of the patient's cancer to treatment with the combination of the BAX activating compound and the anti-apoptotic inhibiting compound and treating the patient if the patient's cancer determined to be sensitive to treatment with the BAX activating/(BCL-XL, BCL-2, or MCL-1) inhibiting combination.

The antitumor activity of chemotherapeutic and targeted agents is a consequence of their induction of apoptosis in cancer cells. Cancer cells suppress apoptosis to promote survival and proliferation by various mechanisms, and as a result, the use of a single therapeutic agent to treat cancer that is refractory to various treatments often results in medium to weak antitumor activity due to ineffective induction of apoptosis. In particular, deregulation of the anti-apoptotic BCL-2 family interaction network ensures cancer resistance to apoptosis and is a significant challenge for current treatments. For the purposes of this discussion “BCL-2 family” includes the anti-apoptotic proteins BCL-XL, BCL-2, BCL-w, BFL-1, BCL-B, and MCL-1. Cancer cells commonly evade apoptosis through upregulation of the BCL-2 anti-apoptotic proteins. More resistant cancers also downregulate or inactivate pro-apoptotic BH3-only proteins to suppress apoptosis.

In consideration of the critical role of anti-apoptotic BCL-2 proteins in apoptosis resistance by cancer cells, and the interactions among BCL-2 family members, selective drugs have been designed to inhibit anti-apoptotic BCL-2 proteins, termed BH3-mimetics. These selective inhibitors of anti-apoptotic BCL-2 proteins (for example, Venetoclax (CAS Reg. No. 1257044-40-8), Navitoclax (CAS Reg. No. 923564-51-6), 563845 (CAS Reg. No.1799633-27-4), S64315 (also MIK665, CAS Reg. No. 1799631-75-6) and AMG176 (Amgen, CAS Reg. No. 1883727-34-1), induce apoptosis primarily by releasing BH3-only proteins (e.g., BIM and BID) from the anti-apoptotic BCL-2 proteins to successively activate BAX and BAK. In preclinical and clinical studies, BH3-mimetics have shown significant efficacy in tumors when cell survival is highly dependent on the targeted anti-apoptotic BCL-2 family protein. However, these molecules have shown limited single-agent activity in many cancers, especially in solid tumors that rely on or upregulate additional non-targeted anti-apoptotic BCL-2 family proteins to ensure survival. Therefore, the full potential of BH3-mimetics to induce tumor apoptosis is yet to be determined by using rational and safe combination treatments to help to overcome resistance mechanisms to apoptosis and the identification of predictive biomarkers for precision therapy.

Direct BAX activation with targeted small molecules offers the potential to overcome the impediment of the downregulation of activator BH3-only proteins in cancer. Based on the understanding that cancer cells contain functional BAX in an inactive cytosolic conformation and only infrequently BAX is mutated or not expressed, the inventors focused on the development of specific BAX activators as a therapeutic strategy in cancer. As a result of these studies, it has been discovered that apoptosis resistance in a diverse range of hematological malignancies and solid tumors is mediated by an unprimed apoptotic state and overexpression of BCL-XL, limiting direct and indirect activation of pro-apoptotic BAX. These survival mechanisms are overcome by the pharmacological combination of BAX activation and BCL-XL inhibition. Additionally, functional assays and genomic markers have been identified to predict tumor sensitivity to the combination treatment. The disclosed findings advance the understanding of apoptosis resistance mechanisms and demonstrate direct BAX activation and BCL-XL inhibition combination as a novel therapeutic strategy for cancer treatment.

In various aspects, it has been surprisingly discovered that the combination of an orally bioavailable BAX activator, BTSA1.2, and Navitoclax, a clinical BCL-XL inhibitor, demonstrates synergistic efficacy in apoptosis-resistant cancer cells, xenografts and patient-derived tumors while sparing healthy tissues.

As used herein, “BAX” refers to BCL-2-associated X-protein. The BAX is a mammalian protein, and in aspects, is a human protein.

The BAX activating compound is a compound which activates cytosolic BAX and/or mitochondrial BAX. The activation of BAX plays a role in initiating cellular apoptosis. The pharmaceutical composition comprises the BAX activating compound in an amount effective to activate BAX in the cell.

In aspects the BAX activating compound is BTSA1, or a pharmaceutically acceptable salt thereof.

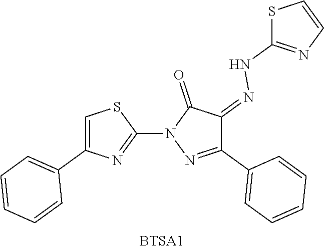 [SMILES: O=C1/C(=N\Nc2nccs2)C(c2ccccc2)=NN1c1nc(-c2ccccc2)cs1]

In aspects the BAX activating compound is a BTSA1.2, or a pharmaceutically acceptable salt thereof.

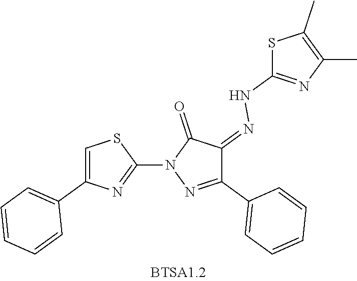

The BAX activating compound can be a compound of Formula A, where Formula A has the structure

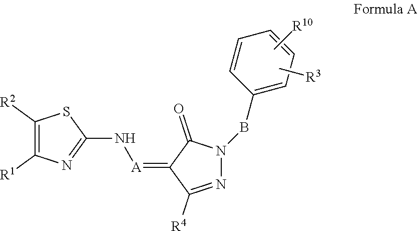

where

A is N or CH:

B is

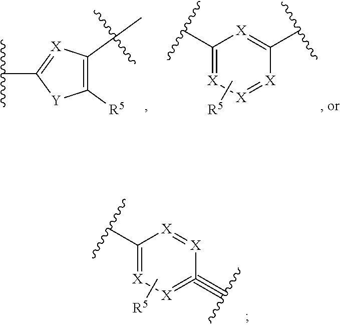

where [symbol] represent the point of attachment to the scaffold’

X is CH or N;

Y is 0, S, NH, CO, CS, or —CH═X;

R¹, R², R⁴, and R⁵ are independently H, F, Cl, Br, I, OH, SH, NO₂, CF₃, COOH, COOR⁶, CHO, CN, NH₂, SO₄H, SO₂NH₂, NHNH₂, ONH₂NHC═(O)NNH₂, NHC═(O)NH₂, NHC═(O)H, NHC(O)—OH, NHOH, OCF₃, OCHF₂, NHR⁶, NHCONH₂, NHCONHR⁶, NHCOR⁶, OCR⁶, COH, COR⁶m CH₂R⁶, CH₂R⁶, CONH², CON(R⁶R⁷), CH═N═OR⁶, CH═NR⁶, OR⁶, SR⁶, SOR6, SO₂R⁶, CH₂N(R⁶R⁷), N(R⁶R⁷), or optionally substituted lower (C₁-C₄)alkyl, alkenyl, alkynyl, cycloalkyl, heterocycloalkyl, aryl, heteroaryl, aralkyl, or heteroaralkyl, where the optional substituent is one or more of F, CF₃, Cl, Br, I, OH, SH, NO₂, R₆, COOH, COOR⁶, CHO, CN, NH₂, NHR⁶, NHCONH₂, NHCONHR₆, NHCOR₆, NHSO₂R₆, OCR⁶, COR⁶, CH₂R⁶, CON(R⁶R⁷), CH═N—OR⁶, CH═NR⁶, OR⁶, SR⁶, SOR⁶, SO₂R⁶, COOR⁶, CH₂N(R⁶R⁷), or N(R⁶R⁷); or

R¹ and R² can form a cyclic, heterocyclic, aryl, or heteroaryl ring, wherein the aryl or heteroaryl ring is optionally substituted with OH, CO₂H, or SO₂NH₂;

R³ and R⁰ are independently H, F, CF₃, Cl, Br, I, OH, SH, CF₃, NO₂, R₆, COOH, COOR⁶, CHO, CN, NH₂, SO₄H, NHNH₂, ONH₂, NHC═(O)NHNH₂, NHC═(O)NH₂, NHC═(O)H, NHC(O)—OH, NHOH, OCF₃, OCHF₂, NHR⁶, NHCONH₂, NHCONHR₆, NHCOR⁶, NHSO₂R⁶, OR⁶, SR⁶, SOR⁶, SO₂⁶, COOR⁶, CH₂N(R⁶, R⁷), N(R⁶, R⁷), lower (C₁-C₄) alkyl, alkenyl, or alkynyl;

R⁶ and R⁷ are independently H, C₁-C₆ alkyl, C₁-C₆haloalkyl, C₃-C₆cycloalkyl, C₁-C₆alkoxy, C₁-C₆haloalkoxy, C₁-C₆thioalkoxy, or C₁-C₆thiohaloalkoxy; or a pharmaceutically acceptable salt thereof.

Other examples of BAX activating compounds are those compounds disclosed in U.S. 2020/0093802, which is incorporated herein by reference in its entirety. Any of the BAX activating compounds disclosed in U.S. 2020/0093802, or a combination thereof, can be included as BAX activating compounds in the combinations disclosed herein.

As used herein, “BCL-XL” refers to B-cell lymphoma-extra large protein. “BCL-2” is B Cell Lymphoma 2 protein. “MCL-1” is Myeloid Cell Leukemia 1 protein. The BCL-XL, BCL-2, and MCL-2 are mammalian proteins, and in aspects, human proteins. The BCL-XL, BCL-2, or MCL-1 proteins are anti-apoptotic proteins, which play a role in inhibiting cellular apoptosis through a number of different mechanisms, one of which is inhibition of BAX. In various aspects, the BCL-XL, BCL-2, or MCL-1 inhibiting compound is a compound which binds to the anti-apoptosis protein (such as a BCL-XL, BCL-2, or MCL-1 protein) thereby inhibiting its function in cellular apoptosis. The BCL-XL, BCL-2, or MCL-1 inhibiting compound can be any compound capable of inhibiting the function, and specifically the anti-apoptotic function, of BCL-XL, BCL-2, or MCL-1 in a cell. The pharmaceutical composition comprises the anti-apoptotic protein inhibiting compound (such as the BCL-XL, BCL-2, or MCL-1 inhibiting compound) in an amount effective to inhibit the anti-apoptotic activity of the anti-apoptotic protein (such as BCL-XL, BXL-2, or MCL-1) in the cell.

Non-limiting examples of the BCL-XL inhibiting compounds include Navitoclax, A1331852, A1155463, a pharmaceutically acceptable salt thereof, or a combination thereof. In aspects, the BCL-XL inhibiting compound is Navitoclax, also known as ABT-263 or as 4-(4-{[2-(4-Chlorophenyl)-5,5-dimethyl-1-cyclohexen-1-yl]methyl}-1-pipera-zinyl)-N-[(4-{[(2R)-4-(4-morpholinyl)-1-(phenylsulfanyl)-2-butanyl] amino}-3-[(trifluoromethyl)sulfonyl]phenyl)sulfonyl]benzamide. The BCL-XL inhibitor can be used alone or conjugated to an antibody capable of targeting a specific cell type. The BCL-XL inhibitor can be linked to an E3 ligase ligand to form a BCL-XL PROTAC degrader , for example DT2216, that can cause BCL-XL protein degradation. (Khan, S., et al., Nature Medicine, (2019) 25: 1938-1947.)

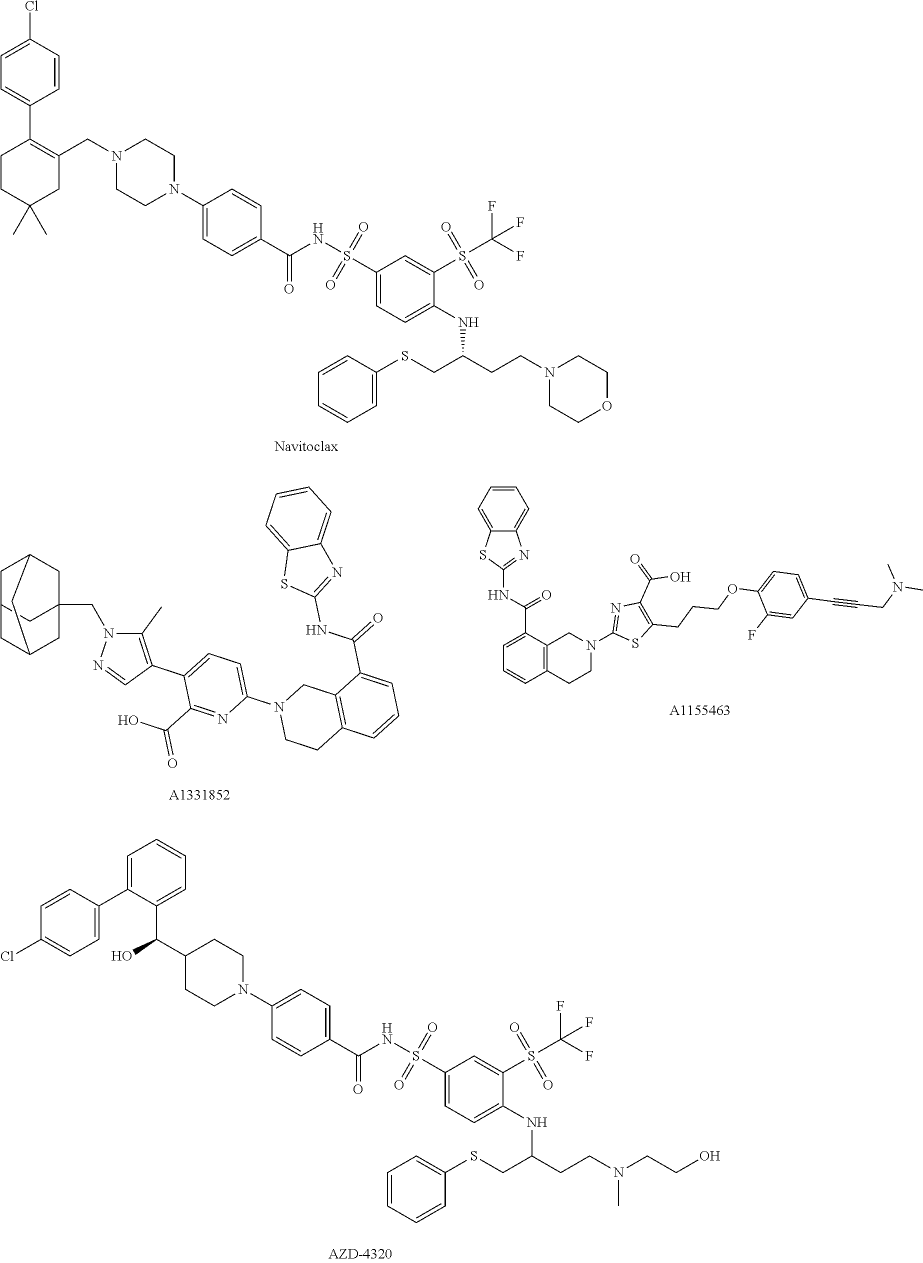

Additional BCL-XL inhibitors that can be used in the combination of this disclosure include AZD0466 (a drug dendrimer conjugate comprising the dual BCL2/XL inhibitor, AZD-4320), AZD-4320 (Astra Zeneca), ABBV-155 (Abbvie), and APG-1252 (Ascentage Pharma), DT2216 (Dialectic Therapeutics).

As used herein the anti-apoptotic protein is an anti-apoptotic protein of the BCL-2 protein family, examples of which include BCL-XL, BCL-2, BCL-w, BFL-1, or MCL-1. Anti-apoptotic protein inhibiting compounds include ABT-737, CAS Reg. No. 852808-04-9, from Abbvie, which binds with high affinity (<1 mol/L) to the BCL-2, BCL-XL, and BCL-w anti-apoptotic proteins; ABT-263 (navitoclax), CAS Reg. No. 923564-51-6, from Abbvie, which binds with high affinity to the BCL-2, BCL-XL, and BCL-w anti-apoptotic proteins; ABT-199 (venetoclax) from Abbvie, CAS Reg. No. 1257044-40-8, which is highly specific for BCL-2, approved for treating hematoloigc cancers including chronic lymphocyctic lymphoma (CLL) including relapsed and refractory CLL, small lymphatic lymphoma (SLL), and acute myeloid leukemia (AML), and is also useful for treating solid tumors; AMG-176, CAS Reg. No. 1883727-34-1, an MCL-1 inhibitor from Amgen, useful for treating multiple myeloma (MM); AMG 397, CAS Reg. No. 2245848-05-7; AZD-4320, CAS Reg. No. 1357576-48-7, a BCL-2 and BCL-XL inhibitor from Astra Zeneca, useful for treating lymphoma: AZD-0466 a BCL-2 and BCL-XL inhibitor from Astra Zeneca and Starpharma, which is a conjugate of AZD-4320 and a Starpharma dendrimer, useful for treating advanced solid tumors, lymphoma, and multiple myeloma; VU661013, CAS Reg. No. 2131184-57-9, an MCL-1 inhibitor from Vanderbilt and Boehringer Ingelheim; 565487 a BCL-2 inhibitor from Servier and Novartis, useful for treating Acute Myeloid Leukema, Multiple Myeloma, and Non-Hodgkin's Lymphoma; 564315 (MIK665), CAS Reg. No. 1799631-75-6 an MCL-1 inhibitor from Servier and Novartis useful for treating multiple myeloma, Non-Hodgkin's Lymphoma, and Multiple Myeloma; and APG1252 (pelcitoclax), CAS Reg. No. 1619923-36-2, a BCL-2, BCL-XL, and BCL-w inhibitor useful for treating Small Cell Lung Cancer (SCLC) and other solid tumors.

The disclosure also includes a combination of a BAX activator and an MCL-1 inhibitor, for example AMG-176 (Amgen), AZD5991 (Astra Zeneca), S64315 (MIK665), and VU661013 (Vanderbilt University).

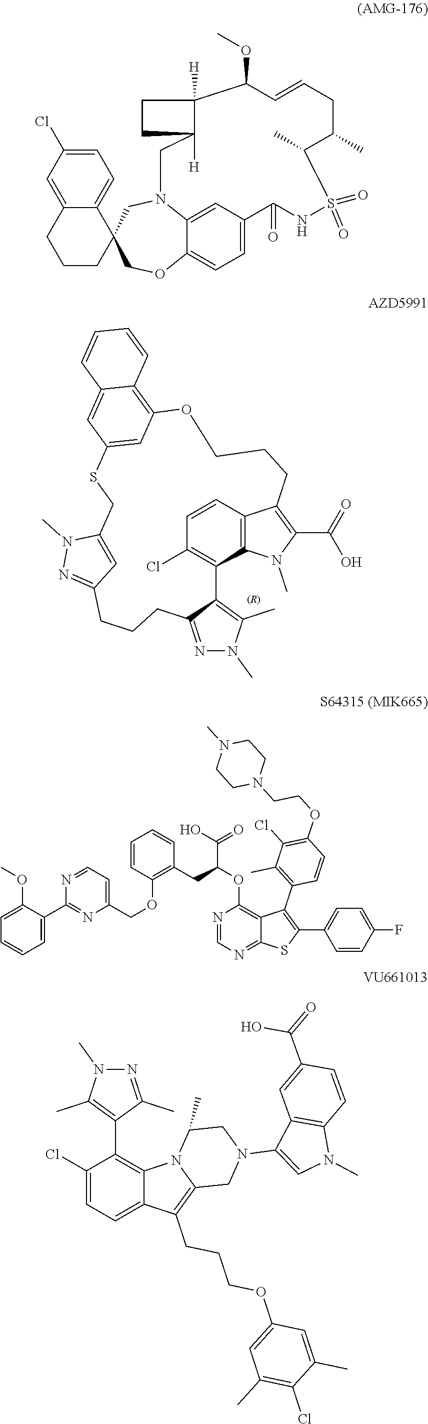

As disclosed herein, the combination of the BAX activating compound with the anti-apoptotic protein inhibitor (such as a BCL-XL, BCL-2, BCL-w, BFL-1, or MCL-1 inhibitor) induces apoptosis in cancer cells (solid cancers and hematological cancers). In aspects, the combination of the BAX activating compound with the anti-apoptotic protein inhibiting compound results in a synergistic treatment effect. In other words, the combination of the BAX activating compound and an anti-apoptotic protein inhibiting compound (such as a BCL-XL, BCL-2, BCL-w, BFL-1, or MCL-1 inhibiting compound) results in improved efficacy as compared to either compound alone. Preferably, the combination of BAX activating compound and the anti-apoptotic protein inhibiting compound allows a treatment effect to be achieved at a lower dose of the anti-apoptotic protein inhibiting compound than in the absence of the BAX activating compound.

In aspects, the combination is a pharmaceutical combination. A “pharmaceutical combination” can be a single pharmaceutical composition containing the BAX activating compound and the anti-apoptoic protein inhibitor, or can be separate pharmaceutical compositions independently comprising the BAX activating compound or the anti-apoptoic protein inhibitor and which are sold together, or packaged together.

The BAX activating compound and the anti-apoptoic protein inhibiting compound can be administered in the form of a composition comprising the compounds, and a pharmaceutically acceptable carrier. In particular, the compounds disclosed herein are administered in the form of a pharmaceutical composition comprising the compounds and a pharmaceutically acceptable carrier. The compounds and compositions can be administered to a subject using any known route of administration. For example, the administration can be systemic or localized to a specific site. Routes of administration comprise, but are not limited to, oral, rectal, sublingual, buccal, intravenous, intramuscular, transdermal, cutaneous, subcutaneous, intrathecal, nasal, vaginal, or a combination thereof. In aspects, the route of administration is oral.

The compounds and compositions are administered to a subject, and in particular, a subject having cancer. The subject is a mammalian subject. The mammalian subject can be, for example, a human, a rodent, a monkey, a cat, a dog, a bovine animal (cow, steer, bull), a sheep, a monkey, or a primate. In aspects, the mammalian subject is a human.

Disclosed herein is method of treating cancer in a subject comprising administering to the subject a BAX activating compound in combination with an anti-apoptotic protein inhibiting compound in an amount effective to treat the cancer in the subject. In aspects, the cancer is a hematological cancer or a solid tumor.

The cancer can be breast cancer, prostate cancer, lymphoma, skin cancer, pancreatic cancer, colon cancer, rectal cancer, colorectal cancer, melanoma, malignant melanoma, ovarian cancer, brain or spinal cord cancer, primary brain carcinoma, medulloblastoma, neuroblastoma, glioma, head-neck cancer, glioma, glioblastoma, liver cancer, bladder cancer, stomach cancer, kidney cancer, placental cancer, cancer of the gastrointestinal tract, small cell lung cancer (SCLC), non-small cell lung cancer (NSCLC), head or neck carcinoma, breast carcinoma, endocrine cancer, eye cancer, genitourinary cancer, cancer of the vulva, ovary, uterus or cervix, hematopoietic cancer, myeloma, leukemia, lymphoma, ovarian carcinoma, lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, chronic granulocytic leukemia, acute granulocytic leukemia, acute myelogenous leukemia, chronic myelogenous leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft tissue cancer, soft-tissue sarcoma, osteogenic sarcoma, sarcoma, primary macroglobulinemia, central nervous system cancer, retinoblastoma, or a combination thereof. In aspects, the cancer is colon cancer, rectal cancer, or colorectal cancer.

In certain embodiments the cancer can be a hematologic cancer, such as non-Hodgkin's lymphoma, multiple myeloma, acute myeloid leukemia, small lymphocytic lymphoma, chronic lymphocyctic lymphoma including recurrent and or refractory chronic lyphocyctic lymphoma.

Also disclosed herein are methods of determining whether the cancer will be sensitive or resistant to treatment with the combination of the BAX activating compound and the BCL-XL inhibiting compound. Functional assays and genomic markers have been advantageously discovered which can be used to predict whether a given cancer is sensitive or resistant to the combination treatment. The detecting comprises quantitative reverse transcription PCR to determine the level of gene messenger RNA in the biological sample.

As disclosed herein, cancer cells which are anti-apoptotic protein dependent or unprimed to apoptosis are sensitive to treatment with the combination of the BAX activating compound and the anti-apoptotic protein inhibiting compound. Accordingly, methods that include determining whether cancer cells in a subject are anti-apoptotic protein dependent or unprimed to apoptosis can be used to determine whether the cancer in the subject having the cancer will respond to treatment upon administration of the combination of the BAX activating compound and the anti-apoptotic protein inhibiting compound. BH3 profiling methods can be used to determine whether the cancer cells are anti-apoptotic protein dependent or unprimed to apoptosis. In aspects, BH3-profiling comprises contacting the cancer cells with a BH3 domain peptide, measuring the amount of BH3 domain peptide induced mitochondrial depolarization in the cancer cells, and comparing the amount of BH3 domain peptide induced mitochondrial outer membrane permeabilization in the cancer cells to a control cell (i.e., non-cancerous cell) population of the same type.

It has also been surprisingly discovered that there is a correlation between the sensitivity of the cancer cell to treatment with the combination of compounds and the formation of BAX:BCL-XL complexes by immunoprecipitation. In particular, it has been discovered that cancer cells which are sensitive to the combination of compounds form higher levels of BAX:BCL-XLcomplexes than cell lines resistant to the combination of compounds. Given the sensitivity of certain cancer cells to other anti-apoptotic protein inhibitors, such as BCL-2, BCL-2, Bfl-2, and MCL-1 it is expected that there is a correlation between the sensitivity of the cancer to treatment with a particular BAX activator/anti-apoptotic protein inhibitor combination and the formation BAX:anti-apoptotic protein complexes detectable by immunoprecipitation. For example detection of BAX:BCL-2 complexes by immunoprecitation is understood to be predictive of sensitivity of the cancer to treatment with a BAX activator/BCL-2 inhibitor combination.

In aspects, a method of determining sensitivity or resistance of a cancer to treatment with an anticancer agent comprising a BAX activating compound in combination with an anti-apoptotic protein inhibiting compound, comprises obtaining a biological sample from a subject having cancer and detecting a level of BAX:BCL-XL, BAX:BCL-2, BAX, BAX:BCL-w, BAX:BFL-1, or BAX:MCL-1 complexes immunoprecipitated from the cancer cells and/or detecting that the cancer cells are anti-apoptotic protein dependent or unprimed to apoptosis. The biological sample from the subject comprises cancer cells.

Detecting that the cancer cells are anti-apoptotic protein dependent or unprimed to apoptosis comprises BH3-profiling of the cancer cells. The BH3-profiling comprises contacting the cancer cells with a BH3 domain peptide and measuring the amount of BH3 domain peptide induced mitochondrial depolarization in the cancer cells and comparing to control cell population of the same type.

The BAX:BCL-XL, BAX:BCL-2, BAX:BCL-w, BAX:BFL-1, or BAX:MCL-1 complexes are formed by co-immunoprecipitation of BAX and anti-apoptotic protein from the cancer cells and are quantified using known methods of determining relative protein expression (e.g., Western blot and band quantification). The method further comprises determining the cancer cells are sensitive to the anticancer agent when the level of BAX:BCL-XL, BAX:BCL-2, BAX:BCL-w, BAX:BFL-1, or BAX:MCL-1 complexes in the cancer cells is increased as compared to normal cells of the same type.

A method of treating cancer in a subject in need thereof comprises: obtaining a biological sample comprising cancer cells from the subject; detecting a level of BAX:BCL-XL, BAX:BCL-2, BAX:BCL-w, BAX:BFL-1, or BAX:MCL-1 complexes immunoprecipitated from the cancer cells and/or detecting that the cancer cells are anti-apoptotic protein dependent or unprimed to apoptosis; and administering to the subject an anticancer agent comprising a B-cell lymphoma 2 associated X protein (BAX) activating compound and an anti-apoptotic protein inhibiting compound (such as a B-cell lymphoma-extra large protein (BC2-XL), BCL-2, BCL-w, BFL-1, or MCL-1 inhibiting compound) in an amount effective to treat the cancer. In aspects, the method further comprises determining the cancer cells are sensitive to the anticancer agent when the level of BAX:BCL-XL BAX:BCL-2, BAX:BCL-w, BAX:BFL-1, or BAX:MCL-1 complexes in the cancer cells is increased as compared to normal cells of the same type.

An analysis of expression of various genetic markers revealed that genes highly expressed in sensitive cancer cell lines include MUC13, EPS8L3, and IGFBP7, and genes highly expressed in resistant cancer cell lines include NR4A3, IRF4, and SLC7A3.

MUC13 gene encodes the protein mucin-13, which is an epithelial and hemopoietic transmembrane mucin. EPS 8L3 gene encodes the epidermal growth factor receptor kinase substrate 8-like protein 3. IGFBP7 gene encodes the insulin-like growth factor-binding protein 7. NR4A3 gene encodes the nuclear receptor subfamily 4, group A, member 3 protein, a transcriptional activator. IRF4 gene encodes interferon regulatory factor 4, a transcriptional activator. SLC7A3 gene encodes cationic amino acid transporter 3, which mediates uptake of arginine, lysine and ornithine in a sodium-independent manner

In aspects, a method of determining sensitivity or resistance of a cancer to treatment with an anticancer agent comprising a B-cell lymphoma 2 associated X protein (BAX) activating compound in combination with an anti-apoptotic protein inhbiting compound (such as a B-cell lymphoma-extra large protein (BCL-XL), BCL-2, BCL-w, BFL-1, or MCL-1 inhibiting compound), comprises obtaining a biological sample from a subject having the cancer; detecting expression level of a gene in the biological sample, wherein the gene comprises MUC13, EPS8L3, IGFBP7, NR4A3, IRF4, SLC7A3, or a combination thereof; and determining that the cancer is sensitive or resistant to the anticancer agent. In certain embodiments the anti-apoptotic protein inhbiting compound is BCL-XL and the gene detected is MUC13, EPS8L3, or IGFBP7. A determination that a cancer cell is sensitive to the combination of the BAX activating compound and the anti-apoptotic protein inhibiting compound can be made when the expression level of the gene MUC13, EPS8L3, IGFBP7, or a combination thereof in the biological sample is increased as compared to a control sample. A determination that a cancer cell is resistant to the combination of the BAX activating compound and the anti-apoptotic protein inhibiting compound can be made when the expression level of the gene NR4A3, IRF4, SLC7A3, or a combination thereof in the biological sample is increased as compared to a control sample.

A method of treating cancer in a subject in need thereof, comprises: obtaining a biological sample from the subject; measuring expression level of at least one gene in the biological sample, wherein the gene comprises MUC13, EPS8L3, IGFBP7, or a combination thereof; and administering to the subject an anticancer agent comprising a B-cell lymphoma 2 associated X protein (BAX) activating compound and an anti-apoptotic protein inhibiting compound in an amount effective to treat the cancer. The method further comprises determining that the expression level of the gene MUC13, EPS8L3, IGFBP7, or a combination thereof in the biological sample is increased as compared to a control sample prior to administering the sample. In aspects, the biological sample comprises cancer cells and the control sample comprises normal cells of the same type. The detecting comprises quantitative reverse transcription PCR to determine the level of gene messenger RNA in the biological sample.

“Pharmaceutical compositions” are compositions comprising an active agent, and at least one other substance, such as an excipient. An excipient can be a carrier, filler, diluent, bulking agent or other inactive or inert ingredients. Pharmaceutical compositions optionally contain one or more additional active agents. When specified, pharmaceutical compositions meet the U.S. FDA's GMP (good manufacturing practice) standards for human or non-human drugs.

“Pharmaceutically-acceptable carrier” refers to a diluent, adjuvant, excipient, or carrier, other ingredient, or combination of ingredients that alone or together provide a carrier or vehicle with which a compound or compounds of the invention is formulated and/or administered, and in which every ingredient or the carrier as a whole is pharmaceutically) acceptable. Also included are any and all solvents, dispersion media, coatings, antibacterial and antifungal agents, and isotonic and absorption delaying agents. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Pharmaceutically acceptable salt” to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC-(CH₂)ₙ—COOH where n is 0-4, and the like.

“Treating,” as used herein includes providing the compounds disclosed herein as either the only active agent or together with at least one additional active agent sufficient to: (a) inhibit the cancer, i.e. arrest its development; and (b) relieve the disease, i.e., causing regression of the cancer.

An “effective amount” of an active ingredient, or a pharmaceutical composition/combination including the active ingredient, is an amount effective, when administered to a subject, to provide a therapeutic benefit.

Throughout this disclosure and in the claims, the open ended transitional phrase “comprising” includes the intermediate transistional phrase “consisting essentially of” and the closed transistional phrases “consists” or “consisting of.” Claims using “comprising” can be amended with the intermediate and closed transitional phrases to designate particular embodiments.

In aspects, one or more additional therapeutic agents can be included in the pharmaceutical composition. Additional therapeutic agents include, for example, agents that induce apoptosis; polynucleotides (e.g., anti-sense, ribozymes, siRNA); polypeptides (e.g., enzymes and antibodies); biological mimetics; agents that bind to and inhibit anti-apoptotic proteins (e.g., agents that inhibit anti-apoptotic proteins such as BCL-2, BCL-XL, BCL-w, BFL-1, or MCL-1 proteins); alkaloids; alkylating agents; antitumor antibiotics; antimetabolites; hormones; platinum compounds; monoclonal or polyclonal antibodies (e.g., antibodies conjugated with anticancer drugs, toxins, defensins, etc.), toxins, radionuclides; biological response modifiers (e.g., interferons (e.g., IFN-.alpha., etc.) and interleukins (e.g., IL-2, etc.), etc.); adoptive immunotherapy agents; hematopoietic growth factors; agents that induce tumor cell differentiation (e.g., all-trans-retinoic acid, etc.); gene therapy reagents (e.g., antisense therapy reagents and nucleotides); tumor vaccines; angiogenesis inhibitors; proteosome inhibitors: NF kappa beta modulators; anti-CDK compounds; and HDAC inhibitors. Agents that induce apoptosis include, for example, radiation (e.g., X-rays, gamma rays, UV); kinase inhibitors (e.g., Epidermal Growth Factor Receptor (EGFR) kinase inhibitor, Vascular Growth Factor Receptor (VGFR) kinase inhibitor, Fibroblast Growth Factor Receptor (FGFR) kinase inhibitor, Platelet-derived Growth Factor Receptor (PDGFR) kinase inhibitor, and Bcr-Abl kinase inhibitors such as GLEEVEC); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; and staurosporine.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

CELL LINES. Cell lines were purchased from ATCC and DSMZ. Head and neck cancer cell lines HN30, HN31, UMSCC6, MDA686LN, and HN5, were provided by Dr. Thomas Ow. Ovarian, NSCLC, Colon, Leukemia, Lymphoma, BxPC-3 and ASPC1 cells lines were maintained in RPMI 1640 media Gibco) supplemented with 10% FBS, 100 U/ml penicillin/streptomycin, 2 mM L-glutamine, and 50 μM β-mercaptoethanol. Breast, Melanoma, HCT116, MIA PaCa-2 and HEY cell lines were maintained in DMEM (Gibco) supplemented with 10% FBS, 100 U ml-1 penicillin/streptomycin and 2 mM 1-glutamine Head and Neck cancer cell lines were maintained in DMEM (Gibco) supplemented with 10% FBS, 1× Vitamins, 1× sodium pyruvate, 1× nonessential amino acids, 100 U ml-1 penicillin/streptomycin and 2 mM L-glutamine Capan-1 was maintained in Iscove's Modified Dulbecco's Medium (Gibco) supplemented with 10% FBS, 100 U ml-1 penicillin/streptomycin and 2 mM 1-glutamine Capan-2 was maintained McCoy's 5a Medium Modified (Gibco) supplemented with 10% FBS, 100 U ml-1 penicillin/streptomycin and 2 mM 1-glutamine OCI-AML3 was maintained in MEM α Gibco) supplemented with 10% FBS, 100 U ml-1 penicillin/streptomycin, 2 mM 1-glutamine and 50 μM β-mercaptoethanol.

### MICE

For toxicity studies, 6-8 week old DUGS male and female mice were purchased from Charles River. For xenograft studies and pharmacodynamics analysis experiments, 6-8 week old nude (nu/nu) mice and NOD SCID male mice were purchased from Charles River. All mice were kept under standard conditions and diet and has a weight of greater than 20 grams.

PATIENT-DERIVED XENOGRAFT SAMPLES. Human colorectal tumor xenografts were obtained from Eduardo Vilar at The University of Texas, MD Anderson Cancer Center. Samples were obtained from two patients with metastatic colorectal cancer (see Table 1 below). Patients provided written informed consent for patient derived xenografts (PDX) under an IRB-approved protocol. Animal experiments using PDXs were performed according to the IACUC-approved protocols.

TABLE 1

| | | Tissue | TNM | | BRAF | KRAS | PIK3CA |
|---|---|---|---|---|---|---|---|
| PDX | Age | Source | Staging | MSS/MSI | mutation | mutation | mutation |
| Colo-1 | 74 | CRC | T4aN0Mx | MSI-H | BRAF_v60 | | |
| | | primary; | | | DE | | |
| | | right | | | | | |
| | | colon | | | | | |
| Colo-2 | 35 | CRC; | T2N1Mx | MSI-H | | KRAS_G13 | PIK3CA_E1 |
| | | liver | | | | D | 12K |
| | | metastases | | | | | |

COMPOUNDS. Hydrocarbon-stapled peptide corresponding to the BH3 domain of BIM, FITC-BIM SAHB_{A2}: FITC-βAla-EIWIAQELRS5IGDS5F\grave{ } NAYYA-CONH₂, where S5 represents the non-natural amino acid inserted for olefin metathesis, was synthesized, purified at >95% purity by CPC Scientific Inc. and characterized as previously described.

BTSA1 and BTSA1.2 compounds were synthesized at the Albert Einstein College of Medicine. BTSA1 was synthesized as previously described in Reyna et al, Cancer Cell. 2017 Oct. 9;32(4):490-505.e10. The synthesis and analytical characterization of BTSA1.2 is described below. Other BAX activators were provided by Chembridge and Molport at >98% purity. Navitoclax was purchased from MedCheM Express (99.97% purity) for in vivo studies and SelleckChem (99.53% purity) for in vitro studies. A-1331852 was purchased from SelleckChem (99.8% purity), Venetoclax was purchased from SelleckChem (99.7%) and Staurosporine (99.61% purity). The following BH3 peptides in Table 2 were purchased from Genscript at >95% purity. Peptides had an acetylation as a N-terminal modification and an amidation as a C-terminal modification.

TABLE 2

| | | SEQ |
|---|---|---|
| Name | Peptide | ID NO |
| hBIM | Ac-MRPEIWIAQELRRIGDEFNA-NH2 | 1 |
| hBID-Y | Ac-EDIIRNIARHLAQVGDSMDRY-NH2 | 2 |
| mBAD | Ac-LWAAQRYGRELRRMSDEFEGSFKGL-NH2 | 3 |
| HRK-y | Ac-SSAAQLTAARLKALGDELHQY-NH2 | 4 |
| mNoxaA | Ac-AELPPEFAAQLRKIGDKVYC-NH2 | 5 |
| Puma | Ac-EQWAREIGAQLRRMADDLNA-NH2 | 6 |
| Bmf-Y | Ac-HQAEVQIARKLQLIADQFHRY-NH2 | 7 |
| Puma2A | Ac-EQWAREIGAQARRMAADLNA-NH2 | 8 |
| MS1 | Ac-RPEIWMTQGLRRLGDEINAYYAR-NH2 | 9 |
| FS1 | Ac-QWVREIAAGLRLAADNVNAQLER-NH2 | 10 |

Compounds were reconstituted in 100% DMSO and diluted in aqueous buffer or cell culture medium for assays.

CHEMICAL SYNTHESIS. All chemical reagents and solvents were obtained from commercial sources (Aldrich, Acros, Fisher) and used without further purification unless otherwise noted. Anhydrous solvents (tetrahydrofurane, toluene, dichloromethane, diethyl ether) were obtained using a Pure Solv™ AL-258 solvent purification system. Ethanol was dried over activated 4 A molecular sieves. Microwave reactions were performed on an Anton Paar Monowave 300. Chromatography was performed on a Teledyne ISCO CombiFlash R_{f} 200i using disposable silica cartridges (4, 12, and 24 g). Analytical thin layer chromatography (TLC) was performed on aluminum-backed Silicycle silica gel plates (250 μm film thickness, indicator F254). Compounds were visualized using a dual wave length (254 and 365 nm) UV lamp, and/or staining with CAM (cerium ammonium molybdate) or KMnO4 stains. NMR spectra were recorded on Bruker DRX 300 and DRX 600 spectrometers. 1H and ¹³C chemical shifts (δ) are reported relative to tetramethyl silane (TMS, 0.00/0.00 ppm) as internal standard or to residual solvent (CD₃OD: 3.31/49.00 ppm; CDCl₃: 7.26/77.16 ppm; dmso-d6: 2.50/39.52 ppm). Mass spectra were recorded on a Shimadzu LCMS 2010EV (direct injection unless otherwise noted). High resolution electrospray ionization mass spectra (ESI-MS) were obtained at the Albert Einstein College of Medicine's Laboratory for Macromolecular Analysis and Proteomics. Or obtained externally from Intertek USA, Inc. (Whitehouse, NJ). Unless otherwise noted the purity of the compounds synthesized was ³ 95% as judged by the 1H-NMR trace.

SYNTHESIS OF BTSA 1.2. The synthesis of BTSA 1.2 is summarized in the reaction scheme shown below.

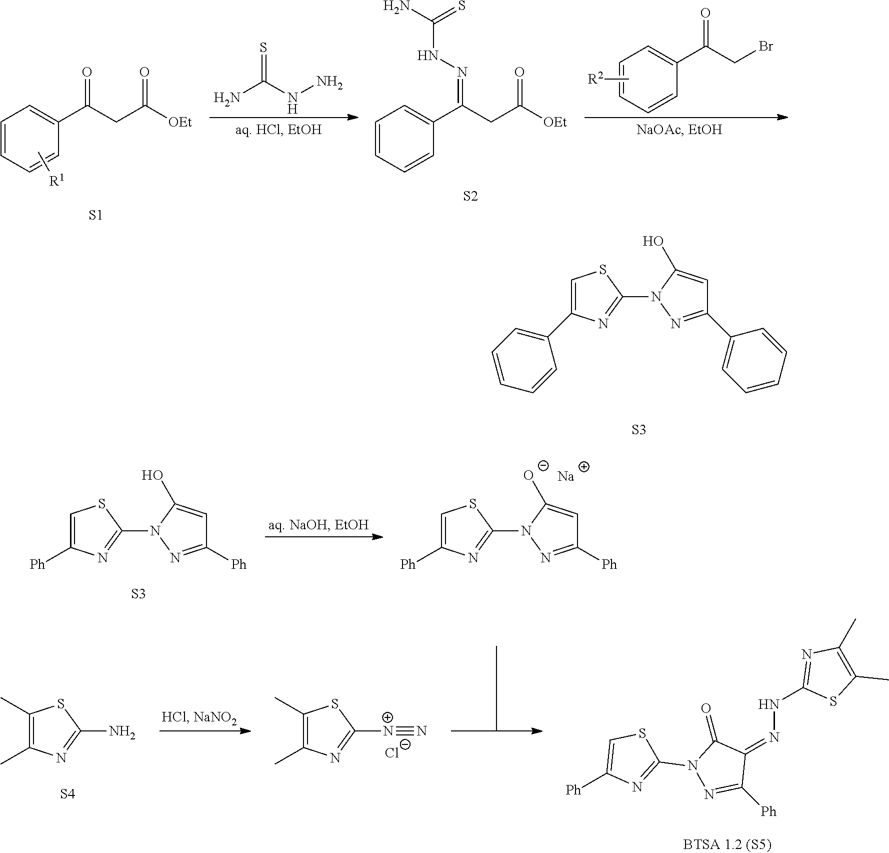

Synthesis of ethyl-3-(2-carbamothioylhydrazono)-3-phenylpropanoate; Compound S2

Hydrazinecarbothioamide (2.00 g, 22.0 mmol, 1.10 equiv) was dissolved in 5% aqueous HCl (64.2 mL, 90 mmol, 4.50 equivalence) and ethanol (30.0 mL). Ethyl 3-oxo-3-phenylpropanoate (3.83 g, 20.0 mmol, 1.00 equiv) was added with vigorous stirring. The resulting mixture was kept stirring vigorously at RT overnight (>12 h). White precipitate that had formed was filtered and washed with little water. The resulting white, fluffy solid (4.24 g) was washed with EtOH (15.0 mL), filtered and dried in high vacuum to obtain ethyl (Z)-3-(2-carbamothioylhydrazono)-3-phenylpropanoate (S2; 3.54 g, 13.3 mmol, 67%, purity ≥90% by 1H-NMR).

1H-NMR (600 MHz, dmso-d6): δ 10.62 (s, 1H), 8.38 (s, 1H), 8.03 (s, 1H), 7.88-7.85 (m, 2H), 7.39-7.37 (m, 3H), 4.10-4.06 (m, 4H), 1.16 (t, J=7.1 Hz, 3H). 13C-NMR (151 MHz, dmso-d6): δ 179.2, 168.3, 142.3, 136.8, 129.2, 128.3, 126.5, 60.8, 33.2, 14.0). The crude product of Compound S2 was used directly in the next step without further purification.

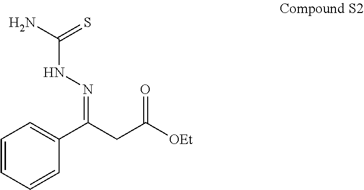 [SMILES: CCOC(=O)C/C(=N/NC(N)=S)c1ccccc1]

Synthesis of 5-phenyl-2-(4-phenylthiazol-2-yl)-1,2-dihydro-3H-pyrazol-3-one; Compound S3

In a 60 mL centrifuge tube with stir bar and cap, ethyl-3-(2-carbamothioylhydra-zono)-3-phenylpropanoate (S2; 1.06 g, 4.00 mmol, 1.00 equiv) and 2-bromo-l-phenylethan-1-one (1.04 g, 5.20 mmol, 1.30 equiv) were suspended in Ethanol (21 mL). The mixture was stirred at RT. Almost immediately, all material dissolved, and ca. 1 min later, a thick, white precipitate formed. After 60 min, sodium acetate (492 mg, 6.00 mmol, 1.50 equiv) was added, and the mixture was kept stirring overnight (>12 h) at RT. 20.0 mL of water were added, the mixture filtered under reduced pressure. Residual material in the reaction vessel was rinsed out with little more water. The residue obtained was washed with more water (total volume of the collected aqueous phases: 60.0 mL). The crude product was obtained as an off white solid that was dried in high vacuum. After purification on the Isco CombiFlash (0.060.0% CH₂Cl₂ in hexanes), 5-phenyl-2-(4-phenylthiazol-2-yl)-1,2-dihydro-3H-pyrazol-3-one (S3) was obtained as colorless solid (850 mg, 2.66 mmol, 71%).

TLC: Rf 0.75 (50% CH₂Cl₂ in hexanes). ¹H--NMR (600 MHz, dmso-d₆+2dr py-d₅): δ 8.03 (d, J=7.3 Hz, 2H), 7.89 (d, J=7.3 Hz, 2H), 7.86 (s, 1H), 7.51-7.45 (m, 5H), 7.37-7.34 (m, 1H), 6.08 (s, 1H). ¹³C-NMR (151 MHz, dmso-d₆+2dr py-d₆): δ 158.5, 155.9, 153.0, 150.1, 133.9, 130.4, 129.7, 128.8, 128.7, 128.0, 126.1, 126.0, 109.8, 88.2. ESI-MS m/z (rel int): (320.1 ([M+H]⁺, 100). HRMS calculated for C₁₈H₁₄N₃OS (M+H) 320.0852, found: 320.0853. (If the NMR is taken in pure dmso-d₆, a mixture of tautomers is observed. Addition of pyridine shifts the equilibrium entirely to one side, allowing for the detection of one distinct set of signals.)

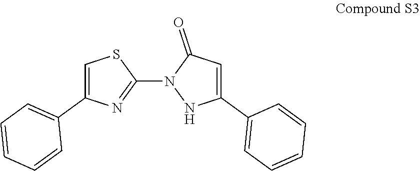 [SMILES: O=c1cc(-c2ccccc2)[nH]n1-c1nc(-c2ccccc2)cs1]

Synthesis of 4-(2-(4,5-dimethylthiazol-2-yl)hydrazono)-5-phenyl-2-(4-phenyl-thiazol-2-yl)-2,4-dihydro-3H-pyrazol-3-one (S5; BTSA 1.2); Compound S5 (BTSA1.2)

To a suspension of 4,5-dimethylthiazol-2-amine (120 mg, 0.939 mmol) in hydrochloric acid (half concentrated, 0.75 mL, 12.4 mmol, 13.2 equiv) in an open vessel in a wet ice/NaCl cooling bath (temperature was kept at £−5° C.) was added drop-wise a solution of sodium nitrite (64.8 mg, 0.939 mmol, 1.00 equiv) in water (0.47 mL) via pipette. The diazonium salt was formed as a deep yellow solution. The starting material had dissolved completely after ca 10 min and the solution was stirred at −5° C. for another 15 min. In parallel, 5-phenyl-2-(4-phenylthiazol-2-yl)-2,4-dihydro-3H-pyrazol-3-one (S3; 300 mg, 0.939 mmol, 1.00 equiv) was dissolved in aqueous sodium hydroxide (2.50 M; 0.94 mL, 2.35 mmol, 2.50 equiv) and ethanol (0.94 mL). A clear solution formed after a few minutes and was stirred for another 10 min at RT. these cases, water/ethanol (1:1) is added in small increments until a solution is formed. The solution of the anionic species formed above was then added to the diazonium salt in a drop-wise dashion. Deep red precipitate formed almost instantaneously. After complete addition, the mixture was warmed to RT and stirred for another 20 min. TLC analysis of a reaction aliquot (either directly diluted in a small amount of methanol or subjected to a H2O/EtOAc micro-workup) indicated consumption of the thiazol amine, formation of a new product, some more polar colored byproducts, as well remaining Compound S3.

The mixture was diluted with water (1.0 mL), filtered into a Buchner funnel with filter paper, washed with little water (ca. 3.0 mL), then dried in the air stream of the filtration apparatus. The pre-dried material was transferred into a flask and dried additionally in high vacuum. After purification on the Isco CombiFlash (0.1@5.0% MeOH in CH₂Cl₃), 4-(2-(4,5-dimethylthiazol-2-yl)hydrazono)-5-phenyl-2-(4-phenyl-thiazol-2-yl)-2,4-dihydro-3H-pyrazik-3-one (BTSA 1.2 S5; 261 mg, 0.569 mmol, 61%) was obtained as a bright red solid.

This synthetic protocol is highly sensitive to mixing/cooling issues, which are complicated by the fact that the product and re-protonated intermediate precipitate during the reaction. In some cases, considerable amounts of impurities, stemming from the decomposition of the diazonium reagent, can be formed. Additional chromatography and/or re-crystallization (most commonly from dioxane) of the product is required in these cases. As a result, yields can be low, despite often acceptable conversions, as judged by TLC.

TLC: Rf 0.90 (5% MeOH in CH2C;2). 1H-NMR (600 MHz, dmso-d6): δ 8.14 (d, J=7.1 Hz, 2H), 7.99 (dd, J=8.1, 1.0 Hz, 2H), 7.80 (s, 1H), 7.54-7.45 (m, 5H), 7.35 (t, J=7.3 Hz, 1H), 2.25 (s, 3H), 2.18 (s, 3H). 13C-NMR (151 MHz, dmso-d6): δ 177.7, 154.7, 152.5, 149.7, 149.2, 134.9, 134.2, 130.9, 129.7, 129.6, 128.7, 128.4, 128.0, 127.9, 125.9, 119.1, 108.8, 11.7, 11.4. ESI-MS (rel int): (459.1 ([M+H]+, 100). HRMS calculated for C₂₂H₁₃N₆O₃S₂ (M+H) 459.1056, found: 459.1051.

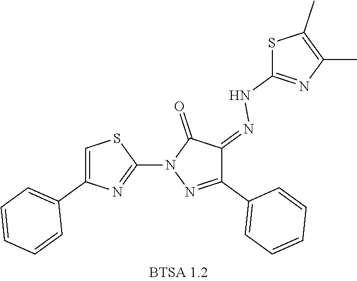 [SMILES: Cc1nc(N/N=C2\C(=O)N(c3nc(-c4ccccc4)cs3)N=C2c2ccccc2)sc1C]

CELL VIABILITY ASSAY. Cancer cells (1-2×103 cells/well) were seeded in 384-well white plates and incubated with serial dilutions of BAX activator compounds including BTSA1.2, Navitoclax, A-1331852, Venetoclax, Staurosporine, or vehicle (1% DMSO) in no FBS media for 2 hrs, followed by 10% FBS replacement to a final volume of 25 μL. Cell viability was assayed at 72 hrs by addition of CellTiter-Glo Assay reagents according to the manufacturer's protocol (Promega), and luminescence measured using a F200 PRO microplate reader (TECAN). For the Navitoclax and BTSA1.2 combination experiments, cells were seeded as described above and co-treated with Navitoclax and BTSA1.2 at the indicated doses. For Navitoclax and BTSA1.2 combination, for A-1331852 and BTSA1.2 combination and Venetoclax and BTSA1.2 combination experiments, cells were seeded as described above and co-treated with Navitoclax or A-1331852 or Venetoclax and BTSA1.2 at the indicated doses. Excluding high-throughput drug screenings, viability assays were performed in at least triplicate and the data normalized to 1% vehicle-treated control wells. IC50 values were determined by nonlinear regression analysis using Prism software (Graphpad). Dilutions of compounds was performed using a TECAN D300e Digital Dispenser from 10 mM stocks. The BLISS calculation was determined using the Combenefit program as previously described by Di Veroli et al., Bioinformatics. 2016 15;32(18):2866-8.

The genomic alternations in the panel of human cancer cell lines tested is shown below in Table 3.

TABLE 3

| | | TP53 | | | |
|---|---|---|---|---|---|
| Tissue | Cell line | status | RAS status | BRAF status | PIK3CA status |
| Colorectal | RKO | WT | WT | Mutant | Mutant |
| | HCT-116 | WT | Mutant | Mutant | Mutant |
| | HT-29 | Mutant | WT | Mutant | Mutant |
| | SW480 | Mutant | Mutant | WT | WT |
| | COLO320 | Mutant | WT | WT | WT |
| | DLD1 | Mutant | Not available | Not available | Not available |
| Breast | Hs-578-T | Mutant | WT | WT | WT |
| | MCF-7 | WT | WT | WT | Mutant |
| | MDA-MB-231 | Mutant | Mutant | Mutant | WT |
| | LM2 | Mutant | Mutant | Mutant | Not available |
| | SK-BR-3 | Mutant | WT | WT | WT |
| Pancreatic | BxPC-3 | Mutant | WT | Mutant | WT |
| | MIA-PaCa-2 | Mutant | Mutant | WT | WT |
| | Capan-1 | Mutant | Mutant | WT | WT |
| | Capan-2 | Negative | Mutant | WT | WT |
| | AsPC-1 | Negative | Mutant | WT | WT |
| NSCLC | NCI-H460 | WT | Mutant | WT | Mutant |
| | HOP-92 | Mutant | Mutant | WT | WT |
| | NCI-H23 | Mutant | Mutant | WT | WT |
| | NCI-H1703 | Negative | WT | WT | WT |
| | NCI-H520 | Mutant | WT | WT | WT |
| | Calu-6 | Mutant | Mutant | WT | WT |
| Melanoma | SK-MEL-28 | Mutant | WT | Mutant | Mutant |
| | SK-MEL-30 | WT | WT | Mutant | WT |
| | A375 | WT | WT | Mutant | WT |
| Lymphoma | Farage | Mutant | WT | WT | WT |
| | SU-DHL-4 | Mutant | WT | WT | WT |
| | SU-DHL-5 | WT | WT | WT | WT |
| | SU-DHL-6 | Mutant | WT | WT | WT |
| | SU-DHL-16 | Negative | WT | WT | WT |
| | Namalwa | Mutant | WT | WT | WT |
| | Pfeiffer | Negative | WT | WT | WT |
| | REC-1 | Mutant | WT | WT | WT |
| Leukemia | SKM1 | Mutant | Mutant | WT | WT |
| | U937 | Mutant | WT | WT | WT |
| | HEL | Mutant | WT | WT | WT |
| | HL60 | Negative | Mutant | WT | WT |
| | | | (NRAS) | | |
| | OCIAML3 | WT | Mutant | WT | WT |
| | | | (NRAS) | | |
| | ML2 | WT | Mutant | WT | WT |

PRODUCTION OF RECOMBINANT BAX PROTEIN. Human, recombinant and tagless BAX was expressed in Escherichia coli and purified as previously reported (Uchime, O. et al. J. Biol. Chem, 291, 89-102 (2016)). BAX wild type was purified by size-exclusion chromatography in a buffer containing 20 mM HEPES pH 7.2, 150 mM KC1, 1 mM DTT. Superdex 75 10/300 GL and 200 10/300 GL (GE Healthcare) columns were used.

FLUORESCENCE POLARIZATION BINDING As SAYS. Fluorescence polarization assays (FPA) were performed as previously described in Gavathiotis, et al., Nat Chem Biol. 2012 Jul. ;8(7):639-4. Firstly, direct binding isotherms were generated by incubating FITC-BIM SAHBA2 (50 nM) with serial dilutions of full-length BAX and fluorescence polarization was measured at 20 min on a F200 PRO microplate reader (TECAN). Subsequently, in competition assays, a serial dilution of small-molecule or acetylated BIM SAHBA2 (Ac-BIM SAHB) was combined with FITC-BIM SAHBA2 (50 nM), followed by the addition of recombinant protein at EC75 concentration, as determined by the direct binding assay (BAX: 500 nM). EC50 and IC50 values were calculated by nonlinear regression analysis of competitive binding curves using Graphpad Prism software. Ki is calculated from IC50 using the following formula Ki=IC50/(1+([L]/Kd), where [L] is the FITC-BIM SAHBA2 concentration and Kd of the FITC-BIM SAHBA2 binding to BAX.

WESTERN BLOTTING. Protein lysates were obtained by cell lysis in 1% NP-40 buffer (50 mM Tris-HCL, 150 mM NaCl, 1 mM EDTA, 10% Glycerol, 1% NP-40, pH 7.50). Protein samples were electrophoretically separated on 4-12% NuPage (Life Technologies) gels, transferred to mobilon-FL PVDF membranes (Millipore) and subjected to immunoblotting. For visualization of proteins with Odyssey Infrared Imaging System (LI-COR Biosciences) membranes were blocked in Odyssey Blocking Buffer (LI-COR Biosciences). Primary antibodies were incubated overnight at 4° C. in a 1:1,000 dilution. After washing, membranes were incubated with an IRdye800-conjugated goat anti-rabbit IgG or IRdye800-conjugated goat anti-mouse IgG secondary antibodies (LI-COR Biosciences) in a 1:10,000 and 1:20,000 dilution, respectively. Proteins were detected with Odyssey Infrared Imaging System. Antibodies were used to detect the following proteins on membrane: BCL-XL (Cell Signaling Cat. 2762), MCL-1 (Cell Signaling Cat. 4572), BAX (Cell Signaling Cat. 2772), BCL-2 (BD. Cat. 610539), BAK (Millipore Cat. 06-536), BIM (Cell Signaling Cat. 2933S), Cleaved Caspase-3 (Cell Signaling Cat. 9664S), Cleaved PARP (Cell Signaling Cat. 5625S), COX-IV (Cell Signaling Cat. 4850S), β-Actin (Sigma Cat. A1978), β-Tubulin (Cell Signaling Cat. 2146S).

WHOLE CELL IMMUNOPRECIPITATION AND IMMUNOBLOTTING. Protein lysates were obtained by cell lysis in 0.2% NP-40 buffer (50 mM Tris-HCL, 150 mM NaCl, 1 mM EDTA, 10% Glycerol, 0.2% NP-40, pH 7.50) Immunoprecipitation was performed in 600 mL of 400 μg of proteins, which was precleared by centrifugation followed by exposure to 12 μL (50% slurry) protein A/G beads (Santa Cruz) at 4° C. for 30 min. Cleared extracts were incubated overnight with 2 μL of anti-BAX antibody (Cell Signaling Cat. 2772). Samples were then exposed to 20 μL (50% slurry) protein A/G beads (Santa Cruz) at 4° C. for 2 hrs and later centrifuged and washed three times with 0.2% NP-40 buffer and boiled in loading buffer (Life Technologies). Protein samples were electrophoretically separated on 4-12% NuPage (Life Technologies) gels, transferred to mobilon-FL PVDF membranes (Millipore) and subjected to immunoblotting. For visualization of proteins with Odyssey Infrared Imaging System (LI-COR Biosciences) membranes were blocked in Odyssey Blocking Buffer (LI-COR Biosciences). Primary antibodies were incubated overnight at 4° C. in a 1:1,000 dilution. After washes, membranes were incubated with an IRdye800-conjugated goat anti-rabbit IgG or IRdye800-conjugated goat anti-mouse IgG secondary antibodies (LI-COR Biosciences) in a 1:10,000 and 1:20,000 dilution, respectively. Antibodies were used to detect the following proteins on membrane: BCL-XL (Cell Signaling Cat. 2762), MCL-1 (Cell Signaling Cat. 4572), BAX (Cell Signaling Cat. 2772), BCL-2 (BD. Cat. 610539), (3-Actin (Sigma, Cat. A1978).

CELLULAR THERMAL SHIFT ASSAYS (CETSA). BxPC3 cells were seeded in a 10-cm dish until ˜85% confluent. The media was removed and replaced with media with no FBS, and cells were treated with 40 μM BTSA1.2 for 15 min at 37° C. The media was removed, and cells were washed once with PBS and harvested using a cell scraper. Cells were resuspended in PBS to 10×106 cells/mL and 50 μL was transferred to PCR tubes. Cells were then heated in a Biorad C1000 Touch Thermal Cycler for 3 min using a temperature gradient (50, 52.1, 55.4, 59.4, 64.9, 69.2, 72.1, and 74° C.). All cells were lysed by three cycles of freeze-thawing using liquid nitrogen. Samples were then centrifuged at 2×10⁴×g for 15 min at 4° C. The supernatants were collected, resolved by SDS-PAGE and analyzed by western blot with an N-terminal BAX antibody (Cell Signaling, 2772S). Results were quantified by densitometric analysis using the Image Studio software and normalized to 25° C. (100%) and blot background (0%).

CELLULAR BAX TRANSLOCATION ASSAY. Cells were seeded and incubated with serial dilutions of BTSA1.2 or vehicle (1% DMSO) in media with no FBS. After 2 hrs, FBS was supplemented to a final concentration of 10%. Following 4 hrs treatment, cells were lysed in 100 μL of digitonin buffer [20 mM Hepes, pH 7.2, 10 mM KCl, 5 mM MgCl2, 1 mM EDTA, 1 mM EGTA, 250 mM sucrose, 0.025% Digitonin (from 5% w/v stock) and complete protease inhibitors cocktail (Thermo-Fisher)] and incubated on ice for 10 min. The supernatants were isolated by centrifugation at 15,000×g for 10 min and the mitochondrial pellets solubilized in 1% Triton X-100/PBS for 1 hr at 4° C. Pellets were solubilized, subjected to a 15,000×rpm spin for 10 min, and 50 ng of protein was mixed with 25 μL LDS/DTT loading buffer. The equivalent fractional e of the corresponding supernatant samples was mixed with 25 μL LDS/DTT loading buffer. The mitochondrial supernatant and pellet fractions were then separated by 4-12% NuPage (Life Technologies) gels, follow by analysis by immunoblotting with anti-BAX antibody (2772S, Cell Signaling), BCL-XL (Cell Signaling Cat. 2762), MCL-1 (Cell Signaling Cat. 4572). COX-IV (Cell Signaling Cat. 4850S) and β-Tubulin (Cell Signaling Cat. 2146S) are used for loading control of mitochondrial and supernatant fractions, respectively.

IMMUNOPRECIPITATION OF DIGITONIN-FRACTIONATED SUPERNATANT AND MITOCHONDRIAL EXTRACTS. Cells (10×10⁶ cells/well) were seeded in 100 mm dishes and incubated with serial dilution concentrations of BTSA1.2 or vehicle (0.2% DMSO) in media with no FBS in a final volume of 5 mL. After 2 hr, FBS was supplemented to a final concentration of 10%. Following 4 hrs treatment, cells were lysed in 100 μL of digitonin buffer [20 mM Hepes, pH 7.2, 10 mM KCl, 5 mM MgCl2, 1 mM EDTA, 1 mM EGTA, 250 mM sucrose, 0.025% Digitonin (from 5% w/v stock) and complete protease inhibitors (Roche Applied Science)] incubated on ice for 10 min. The supernatants were isolated by centrifugation at 15,000×g for 10 min and the mitochondrial pellets solubilized in NP-40 lysis buffer (50 mM Tris-HCL pH 7.4, 150 mM NaCl, 5 mM MgCl_{2, 1} Mm EGTA, 10% Glycerol, 0.2% NP-40). Immunoprecipitation was performed in 600 μL of 500 μg of proteins from supernatant and mitochondrial pellet fractions. Briefly, fractions were pre-cleared by centrifugation after expose with 12 μL (50% slurry) protein A/G beads (Santa Cruz) at 4° C. for 1 hr. Cleared extracts were incubated overnight with 1 μL of anti-BAX antibody (Cell Signaling Cat. 2772). Samples were then exposed to 20 μL (50% slurry) protein A/G beads (Santa Cruz) at 4° C. for 3 hrs and later centrifuged and washed three times (3,000 g for 1 minute) with NP-40 lysis buffer and boiled in loading buffer (Life Technologies) for 15 min. Protein samples were electrophoretically separated on 4-12% NuPage (Life Technologies) gels, transferred to mobilon-FL PVDF membranes (Millipore) and subjected to immunoblotting. For visualization of proteins with Odyssey Infrared Imaging System (LI-COR Biosciences) membranes were blocked in PBS containing 5% dry milk. Primary antibodies were incubated overnight at 4° C. in a 1:1,000 dilution. After washes, membranes were incubated with an IRdye800-conjugated goat anti-rabbit IgG or IRdye800-conjugated goat anti-mouse IgG secondary antibodies (LI-COR Biosciences) in a 1:5,000 dilution for 1 hr. Antibodies were used to detect the following proteins on membrane: BCL-XL (Cell Signaling Cat. 2764S), MCL-1 (Cell Signaling Cat. 4572), BAX (Cell Signaling Cat. 2772), β-Tubulin (Cell Signaling Cat. 86298S), and COX IV (Cell Signaling Cat. 11967S).

WESTERN BLOT PROTEIN QUANTIFICATION AND PEARSON CORRELATION. Densitometry of protein bands were acquired using a LI-COR Odyssey scanner. Quantification and analysis were performed using the Western Analysis tool from the Image Studio software. Relative expression levels were quantified based on protein expression of respective loading control: COX-IV, β-Actin or β-Tubulin. Pearson correlation was determined using Prism software (Graphpad) comparing the cell viability IC50 for single agents and the combination of Navitoclax and BTSA1.2 values with the protein quantification for different members of the BCL-2 family of proteins.

BH3 PROFILING. Cancer cell lines were compared by BH3 profiling under basal conditions. BIM BH3, BID BH3, BMF-γ, PUMA, BAD, HRK-γ, and NOXA peptides (final concentrations of 10 μM); Puma2A peptide (final concentration of 20 μM); alamethicin (final concentration of 25 μM); CCCP (final concentration of 10 μM) were added to JC1-MEB staining solution (150 mM mannitol, 10 mM HEPES-KOH, 50 mM KCl, 0.02 mM EGTA, 0.02 mM EDTA, 0.1% BSA, 5 mM succinate, pH 7.5) in a black 384-well plate. Single cell suspensions were prepared in JC-1-MEB buffer, as previously described in Montero et al., Cell. 2015 February;160(5):977-89. Cells were kept at room temperature for 10 min to allow for cell permeabilization and dye equilibration. After adding the cells to the 384-well plate, 1.0×10⁴ cells/well to 2.0×10⁴ cells/well, fluorescence was measured at 590 nm emission 545 nM excitation using the M1000 microplate reader (TECAN) at 30° C. every 15 min for a total of 3 hrs. Percentage of depolarization was calculated by normalization to the AUC of solvent-only control DMSO (0% depolarization) and the positive control CCCP (100% depolarization), as previously described by Ryan et al. Methods. 2013 Jun;61(2):156-64.

CASPASE 3/7 ACTIVATION ASSAY. Cancer cells were treated with BTSA1.2, BTSA1, Navitoclax, Venetoclax, or Staurosporine at the indicated concentrations, as single agents or in combination as previously described in the cell viability assays. Caspase-3/7 activation was measured at 8 hrs for BTSA1.2 and Navitoclax and at 24 hrs for Staurosporine by addition of the Caspase-Glo 3/7 chemiluminescence reagent in accordance with the manufacturer's protocol (Promega) Luminescence was detected by a F200 PRO microplate reader (TECAN). Assays were performed at least in triplicate.

PHARMAKOKINETIC ANALYSIS. ICR (CD-1) male mice were fasted at least 3 hrs and water was available ad libitum before the study. Animals were housed in a controlled environment, target conditions: temperature 18-29° C., relative humidity 30 to 70%. Temperature and relative humidity were monitored daily. An electronic time-controlled lighting system was used to provide a 12 hr light/12 hr dark cycle. 3 mice for each indicated time point were administered BTSA1.2 in 1% DMSO, 30% PEG-400, 65% D5W (5% dextrose in water), 4% Tween-80 either by an oral gavage (3 mg/Kg) or intravenous injection (1 mg/Kg). Mice were sacrificed, and plasma samples were harvested at 0 hr, 0.25 hr, 0.5 hr, 1 hr, 2 hrs, 4 hrs, 8 hrs, 24 hrs and analyzed for BTSA1.2 levels using LC-MS/MS. Pharmacokinetics parameters were calculated using Phoenix WinNonlin 6.3. Experiments performed at SIMM-SERVIER joint Biopharmacy Laboratory.

MAXIMUM TOLERATED DOSE (MTD) AND IN VIVO TOXICITY STUDIES. 6-8 weeks old CD1-IGS female and male mice (Charles River) were divided into six groups (n=6 per arm), and treated with vehicle, 200 mg/kg BTSA1, 50 mg/kg, 100 mg/kg, 200 mg/kg or 300 mg/kg BTSA1.2 by oral gavage daily for 5 days. Mice were monitored daily and body weight was monitored at the indicated days. After 14 days of the first treatment, mice were subject to euthanasia and necropsy (Histology and Comparative Pathology Facility, Albert Einstein College of Medicine) and tissues (e.g. spleen, liver, kidney, lung, heart) were harvested for fixation in 10% buffered formalin (Fisher Scientific) for pathology analysis. Paraffin-embedded sections (5 mm) were stained with H&E. Peripheral blood from CD1-IGS mice was obtained by facial vein puncture and collected in EDTA-coated tubes (BD cat. 365973). Blood counts were determined on a Forcyte Veterinary Hematology Analyzer (Oxford Science Inc.). 200 mg/kg BTSA1 and 300 mg/kg BTSA1.2 mice were subjected to necropsy studies, which determined they die by kidney failure after 3 days of treatment. Histological evaluation of tissues and necropsy performed by the Histology and Comparative Pathology Facility board-certified veterinary pathologist.

BTSA1.2 AND NAVITOCLAX COMBINATION IN VIVO TOXICITY STUDIES. 6-8 weeks old CD1-IGS male mice were purchased from Charles River. Mice were divided into four groups (vehicle, BTSA1.2 and Navitoclax n=5, combination n=6), and treated with vehicle, 200 mg/kg BTSA1.2, 100 mg/kg Navitoclax or BTSA1.2 and Navitoclax combination by oral gavage daily for 7 days. Mice in the combination group were first administered with 100 mg/kg Navitoclax and after 6-8 hrs were administered 200 mg/kg BTSA1.2. Mice were monitored daily; body weight and peripheral blood counts were monitored at the indicated days. After 14 days of the first treatment, mice were subject to euthanasia and necropsy (Histology and Comparative Pathology Facility, Albert Einstein College of Medicine) and tissues (e.g. spleen, liver, kidney, lung, heart, bone marrow, brain) were harvested for fixation in 10% buffered formalin (Fisher Scientific) for pathology analysis. Paraffin-embedded sections (5 mm) were stained with H&E. Peripheral blood from CD1-IGS mice was obtained by facial vein puncture and collected in EDTA-coated tubes (BD cat. 365973). Blood counts were determined on a Forcyte Veterinary Hematology Analyzer (Oxford Science Inc.).

TUMOR XENOGRAFT STUDIES. 6-8 weeks old nu/nu nude male mice were purchased from Charles River. Approximately, 2.5×106 SW480 cells were suspended in cold PBS and injected subcutaneously into the right flanks of mice. Mice were divided into four groups (Efficacy study: vehicle, BTSA1.2 and Navitoclax n=5, combination n=6; Pharmacodynamic study: n=3 for all groups), and treated with vehicle, 200 mg/kg BTSA1.2, 100 mg/kg Navitoclax or BTSA1.2 and Navitoclax combination by oral gavage daily. Mice in the combination group were first administered with 100 mg/kg Navitoclax and after 6-8 hrs were administered 200 mg/kg BTSA1.2. For efficacy the efficacy study, treatments started once tumors reached a volume of ˜200 mm³. Tumor volume was monitored every 3 days by caliper measurements until the cessation of the experiment when tumors reached an ethically unacceptable size for the vehicle, BTSA1.2 or Navitoclax treated mice, for the mice administered the combination mice were euthanized the day after single agents or vehicle treated mice were euthanized. Body weight of mice were monitored during treatment. For the pharmacodynamic study, treatments started once tumors reached a volume of ˜400 mm³, after 3 days of daily treatment mice were euthanized and tumors were collected for analysis.

PATIENT-DERIVED XENOGRAFT STUDIES. 6-8 weeks old NOD SCID male mice were purchased from Charles River. Approximately, 1.0×106 COLO-1 or COLO-2 cells were suspended in a 1:1 DMEM:matrigel and injected subcutaneously into the right flanks of mice. PDX characterization: mice were divided into two groups COLO-1 and COLO-2 (n=3) were divided into two groups. Tumor was collected once tumor reached a volume of ˜1,000 mm³. COLO-1 efficacy study: Mice were divided into four groups (vehicle, BTSA1.2, Navitoclax and combination n=4) and treated with vehicle, 200 mg/kg BTSA1.2, 50 mg/kg Navitoclax or BTSA1.2 and Navitoclax combination by oral gavage daily. Mice in the combination group were first administered with 50 mg/kg Navitoclax and after 6-8 hrs were administered 200 mg/kg BTSA1.2. Treatments started once tumors reached a volume of ˜200 mm³. Tumor volume was monitored every 3-4 days by caliper measurements until the cessation of the experiment when tumors reached an ethically unacceptable size or after 18 days of daily treatment (whichever came first). Body weight of mice were monitored during treatment.

Ex-Vivo BHS PROFILING. SW480 xenograft vehicle and combination treated tumors, and COLO-1 and COLO-2 PDX tumors were analyzed by BH3 profiling under basal conditions. Single cells from tumors were isolated by mechanically pass them through a 70 μM strainer filter with cold PBS. SW480 xenograft tumors: BIM BH3 and BID BH3, peptides (final concentrations of 10-0.5 μM); Puma2A peptide (final concentration of 10 μM); alamethicin (final concentration of 25 μM); CCCP (final concentration of 10 μM) were added to JC1-MEB staining solution (150 mM mannitol, 10 mM HEPES-KOH, 50 mM KCl, 1 mM EGTA, 1 mM EDTA, 0.1% BSA, 5 mM succinate, pH 7.5) in a black 384-well plate. PDX tumors: BIM BH3 and BID BH3, peptides (final concentrations of 25-1 μM); PUMA, BMF-γ, BAD and HRK (final concentrations of 100-10 μM); MS1 and FS1 (final concentrations of 25-10 μM); and PUMA2A peptide (final concentration of 100-25 μM); alamethicin (final concentration of 25 μM); CCCP (final concentration of 10 μM) were added to JC1-MEB staining solution in a 384-well plate. Single cell suspensions were prepared in 1:1 JC-1-MEB buffer, as previously described, and were kept at room temperature for 10 min to allow for cell permeabilization and dye equilibration. After adding the cells to the 384-well plate, 2.0×104 cells/well, fluorescence was measured at 590 nm emission 545 nM excitation using the M1000 microplate reader (TECAN) at 30° C. every 15 min for a total of 2 hrs. Percentage of depolarization was calculated by normalization to the AUC of negative control Puma2A (0% depolarization) and the positive control CCCP (100% depolarization), as described above. Mitochondria membrane potential was calculated by normalization of the AUC values to the AUC of negative control solvent-only 1% DMSO.

Ex-Vivo CELL VIABILITY. Single cells from COLO-1 and COLO-2 PDX tumors were isolated by mechanically pass them through a 70 μM strainer filter with cold PBS. Isolated cells (10-20×103 cells/well) were seeded in 384-well white plates and incubated with vehicle (1% DMSO) or serial dilutions of BTSA1.2, Navitoclax, or co-treated with Navitoclax and BTSA1.2 (at the indicated doses) in no FBS media for 2 hrs, followed by 10% FBS replacement to a final volume of 25 μL. Cell viability was assayed at 24 hrs by addition of CellTiter-Glo Assay reagents according to the manufacturer's protocol (Promega), and luminescence measured using a F200 PRO microplate reader (TECAN). Viability assays were performed in at least duplicate and the data normalized to 1% vehicle-treated control wells. IC50 values were determined by nonlinear regression analysis using Prism software (Graphpad). Dilutions of compounds was performed using a TECAN D300e Digital Dispenser from 10 mM stocks.

BIOINFORMATIC ANALYSIS. We tested our drug candidates BTSA1.2 and Navitoclax separately and in combination on a total of 46 cancer cell lines. Cancer cell lines were defined as synergistic or non-synergistic to combination by the fold change from IC50 of Navitoclax to IC50 of Navitoclax and BTSA1.2 combined. Two groups were defined: (A) Synergistic group, IC50 fold change >=4; (B) Non-synergistic group, IC50 fold change <2. RNA-Seq raw counts data were retrieved from the Cancer Cell Line Encyclopedia (CCLE) database at BROAD Institute (https://portals.broadinstitute.org/ccle/data). A total of 23 cell lines (8 non-synergistic and 15 synergistic) have RNA-Seq data from CCLE. Differential expression analysis was then conducted using DESeq2 package in R comparing non-synergistic to synergistic group based on raw RNA-Seq data. Heatmap was generated for the top 150 differentially expressed genes comparing non-synergistic to synergistic cell lines group using pheatmap package in R. A literature search was done for bioinformatic analysis top hits, based on adjusted p-value, for genes which have been previously associated with apoptosis, cancer treatment resistance, BCL-2 family or poor prognosis in cancer. After literature evaluation 8 top hits where selected for further validation by RT q-PCR.

RNA PREPARATION AND REAL-TIME PCR. RNA from cells in culture was isolated using the E.Z.N.A total RNA Kit from Omega, following the manufacturer's instructions. The quality and quantity of the RNA was determined by spectrophotometry using the NanoDrop 8000 Spectrophotometer from Thermo Scientific. For quantitative reverse transcription PCR (RT-qPCR), the RNA was reverse transcribed using the High-Capacity cDNA Reverse Transcription kit from Applied Biosystems, following the manufacturer's instructions. PCR was performed using the PowerUp SYBR Green Master Mix from Applied Biosystems, on a ViiA 7 Real-Time PCR system from Applied Biosystems, following the manufacturer's instructions. The cycling conditions included uracil-DNA glycosylase (UDG) activation for 2 min at 50° C., then activation of the Dual-Lock Taq DNA polymerase for 2 min at 95° C., followed by 40 amplification cycles consisting of 15s of denaturation at 95° C., 15 s of annealing at 60° C., and 1 min of extension at 72° C. The specificity of the amplified DNA was confirmed by performing a melting curve at the end of each RT-qPCR run. No template controls, containing all reaction components except the cDNA sample, were used to identify PCR contamination as this samples should not return a CT value. Gene expression results were normalized to the transcript amount of the ribosomal protein RPL27. The primers used for PCR were designed using the online NCBI Primer-BLAST tool. Each RT-qPCR was performed in at least triplicate. The following primers (Table 4) were purchased from Eurofins Genomics.

TABLE 4

| Gene | Forward Primer | Reverse Primer |
|---|---|---|
| BCL2L1 | TGCAGGTATTGGTGAGTCGG | ACAAAAGTATCCCAGCCGCC |
| | (SEQ ID NO 11) | (SEQ ID NO 12) |
| EPS8L3 | CCTACCAACCCACATTCTCAG | TCCCTAACCTATGACTTCCCC |
| | (SEQ ID NO 13) | (SEQ ID NO 14) |
| IGFBP7 | TGCCATGCATCCAATTCCCA | TATAGCTCGGCACCTTCACCTTT |
| | (SEQ ID NO 15) | (SEQ ID NO 16) |
| IRF4 | GTGAAAATGGTTGCCAGGTGA | AGGCTTCGGCAGACCTTATG |
| | (SEQ ID NO 17) | (SEQ ID NO 18) |
| MUC13 | GTAACCAGACTGCGGATGACT | AGACTGGAAGCAACGCAGAAA |
| | (SEQ ID NO 19) | (SEQ ID NO 20) |
| NR4A3 | GCTGGGCAGAAAAGATTCCG | CAGCAGTGTTTGACCTGATGG |
| | (SEQ ID NO 21) | (SEQ ID NO 22) |
| RPL27 | CATGGGCAAGAAGAAGATCG | TCCAAGGGGATATCCACAGA |
| | (SEQ ID NO 23) | (SEQ ID NO 24) |
| SLC7A3 | TAAGACTCTGCAGGGGTCCA | CCGAGAGCCAACAATCCAGT |
| | (SEQ ID NO 25) | (SEQ ID NO 26) |

CANCER PATIENT GENE EXPRESSION. Survival and gene expression data for genes from cancer patients was obtained from cbioportal curated set of non-redundant studies.

QUANTIFICATION AND STATISTICAL ANALYSIS. Plots and statistical tests were generated in GraphPad Prism 8.0. Data are presented as means±SEM except where noted. Statistical comparisons between two groups were performed using an unpaired one-tailed Student's t-tests or ANOVA with Tukey's multiple comparisons test. P values indicated on the graphs: *p<0.05, **p<0.01, ***p<0.001, ****p<0.0001. Additional statistical details are provided in figure legends and methods details.

### RESULTS

### BAX ACTIVATION IS REGULATED BY BCL-XL AND ADOPTOTIC PRIMING IN RESISTNT CANCER CELLS

To improve biological activity and in vivo properties of the small molecule BAX activator, BTSA1, we performed further medicinal chemistry optimization. A new orally bioavailable analogue, BTSA1.2, which has two methyl groups on the thiazole group of BTSA1, was generated to increase van der Waals contacts with the BAX trigger site based on the previously determined binding pose of BTSA1 (Table 5). In addition, the two methyl groups were installed to avoid potential generation of the reactive and toxic metabolite aminothiazole from in vivo metabolism of BTSA1. BTSA1.2 has the phenyl attached to the pyrazolone group as BTSA1, which provided significantly increased binding to BAX and apoptotic activity compared to BAM7 and Compound 3 that lack this phenyl group (Table 5). BTSA1.2 has the thiazolhydrazone moiety as Compound 3 showed thiazolhydrazone improved binding compared to the ethoxy phenylhydrazone of BAM7 (Table 5). Installing a carboxylic acid to the phenylhydrazone of Compound 5 and to the phenylthiazol of Compound 6 provided less active compounds compared to BTSA1.2 and BTSA1 suggesting that hydrophobic groups are better tolerated to these two rings (Table 5).

TABLE 5
Structure-activity relationships of BAX activators based in vitro binding and
cellular activity.

| Compound | 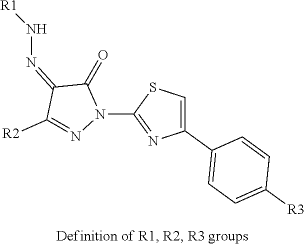 | BIM SAHB- BAX Competition- FPA IC₅₀ (nM) | Viability Inhibition- SU- DHL5- IC₅₀ (μM) |
|---|---|---|---|
| BTSA1.2 | 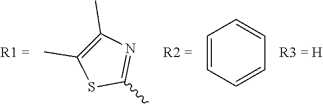 | 109 | 0.23 |
| BTSA1 | 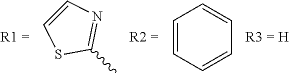 | 159 | 0.40 |
| BAM7 | 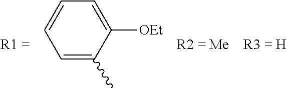 | 3483 | >25 |
| Compound 3 | 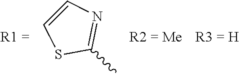 | 2378 | >25 |
| Compound 5 | 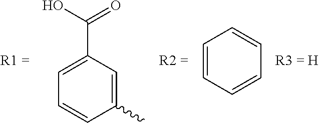 | 497 | 2.8 |
| Compound 6 | 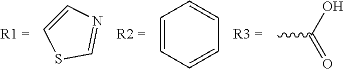 | 500 | 16.1 |

BTSA1.2 demonstrated increased binding to BAX and more potent cellular activity in a set of lymphoma cell lines compared to BTSA1 (Table 5, FIGS. 1L-1O). Moreover, a significant increase in the melting temperature of cellular BAX using a Cellular Thermal Shift Assay (CETSA) assay provided evidence of BTSA1.2 directly engaging with cellular BAX (FIGS. 1P-1Q). Pharmacokinetic analysis of BTSA1.2 demonstrated favorable properties by oral administration such as substantial half-life (T_{1/2}˜14 hr) in mouse plasma, favorable oral bioavailability (% F ˜50%) and significant plasma exposure (AUC ˜100 μM hr) (FIGS. 16A-16B-1S and Table 6). Comparison of BTSA1.2 and BTSA1 in vivo by oral administration of 200 mg/Kg daily dose also confirmed that BTSA1.2 is better tolerated by oral administration as mice survived 5 daily doses without obvious toxic effects. In contrast, mice on BTSA1 died after three daily doses by kidney failure and on the second day mice showed increased white blood cells and neutrophils levels (FIGS. 16F-16G). Thus, BTSA1.2, a rationalized BTSA1 analogue, has improved binding to BAX, cellular cytotoxicity and is better-tolerated in vivo.

TABLE 6
Pharmacokinetic parameters of BTS1.2 after p.o. and i.v. administration in mice.

| | | T_{1/2} | Tₘₐₓ | Cₘₐₓ | AUCₗₐₛₜ | AUC_{inf}_obs | CL_obs | MRT_{INF}_obs | V_{SS}_obs | F(%) |
|---|---|---|---|---|---|---|---|---|---|---|
| PO | Mean | 14.405 | 3.333 | 3686.667 | 47003.131 | 70044.402 | — | 21.683 | — | 49.2 |
| | SD | 2.714 | 1.155 | 1409.302 | 6462.939 | 1551.231 | — | 4.433 | — | |
| IV | Mean | 24.640 | 2.000 | 2673.000 | 31866.471 | 67659.200 | 0.282 | 35.729 | 555.803 | |
| | SD | 8.513 | 0.000 | 593.745 | 8426.532 | 28220.741 | 0.131 | 12.900 | 155.903 | |

Direct and indirect BAX activation is regulated by BCL-XL and apoptotic priming in apoptosis resistant solid tumors. The ability of BTSA1.2 to promote cytotoxicity in a diverse panel of solid and hematological tumor cell lines (n=46) was evaluated. This panel includes including non-small cell lung cancer (NSCLC), breast, head and neck, colorectal, pancreatic, melanoma, ovarian, leukemia, and lymphoma cancer cell lines which contain common genomic alterations in cancer, including mutations of TP53, RAS, BRAF, and/or PIK3CA. BTSA1.2 treatment showed significantly better cytotoxicity in leukemia and lymphoma cell lines (mean IC₅₀<3 μM) than in most solid tumor cell lines (mean IC₅₀>10 μM) (FIG. 1A). Thus, similar to other BH3 mimetics as Venetoclax and S63845, BTSA1.2 showed better efficacy as a single agent in hematological malignancies (Souers et al, Nat Med. 2013 February;19(2):202-8; Kotschy et al., Nature. 2016 Oct. 19;538(7626):477-82; Tron et al, Nat Commun. 2018 December;9(1):5341). However, to fully explore the potential of BTSA1.2, the use of rational and safe combination treatments could help overcome resistance to direct BAX activation.

It was hypothesized that anti-apoptotic BCL-2 proteins could promote resistance to BAX activator treatment. Quantitative protein expression profiling of key BCL-2 family proteins was conducted in the diverse panel of cancer cell lines and showed a strong correlation of BTSA1.2 cytotoxicity with BAX expression levels (FIG. 1B, FIGS. 9A-9D). Interestingly, among key BCL-2 family proteins only anti-apoptotic BCL-XL). Interestingly, among key BCL-2 family proteins only anti-apoptotic BCL-XL levels correlated with the less potency of BTSA1.2, suggesting that BCL-XL is a key protein promoting resistance to the BAX activator treatment (FIG. 1B). To assess the role of BCL-XL to the BTSA1.2 treatment, we examined whether BAX is activated in solid tumor cell lines BxPC-3 and SW80, which were less sensitive to BTSA1.2 in the panel of cell lines. Cytosolic BAX translocation to the mitochondria was achieved at 10 μM BTSA1.2 in BxPC-2 cells and in SW480 cells at 4 hr (FIGS. 1C-1D, FIG. 11A, 11B). However, a lower concentration of BTSA1.2 (2.5-5 μM) also promoted cytosolic BAX translocation to the mitochondria in BxPC-3 cells at later time point of 18 hrs (Supplemental FIG. 11C). Interestingly, co-immunoprecipitation of BAX in SW480 and BxPC-3 cells showed BAX:BCL-XL complexes were formed without BTSA1.2 treatment and only BAX:BCL-XL complexes but not BAX:MCL-1 complexes increased upon BTSA1.2 treatment. (FIG. 1D and FIGS. 11D, 11E) Moreover, complexes of BAX:BCL-XL were detected in several solid tumor cell lines without BTSA1.2 treatment (Supplementary FIGS. 11F, 11G). Taken together, this data suggested that BRSA1.2 of inducing BAX activation and translocation but the onset of BAX-mediated apoptosis may be hindered by the interaction of BCL-XL with BAX.

BCL-XL was suggested as a factor of resistance to direct BAX activation in our panel of cancer cell lines, we examined whether Navitoclax, a clinical BCL-XL/BCL-2 inhibitor, has efficacy to promote cytotoxicity against the same panel of cancer cell lines. Of note, BCL-2 inhibition by Navitoclax should not account for the decrease of cell viability as only a small portion of solid tumors cell lines had detectable levels of BCL-2 protein (FIG. 9A). Interestingly, several solid tunmor cell lines that were found to be resistant to BTSA1.2 treatment, were also less responsive to BCL-XL inhibition by Navitoclax, which was not sufficient to decrease cell viability in several of these cell lines (mean IC₅₀>10 μM) (FIG. 1E, FIG. 10A-10B). Analysis of key BCL-2 family proteins expression showed that higher MCL-1 and BCL-XL levels correlated with resistance to Navitoclax treatment (FIG. 1F, FIG. 9A, 10C). On the other hand, BAX:BCL-XL ratio correlated with Navitoclax sensitivity, suggesting that cells with higher expression of BAX will, in general, be more sensitive to BCL-XL inhibition (FIG. 1F, FIG. 10C). Collectively, these data suggested that BCL-XL inhibition as a single agent is not adequate to promote apoptosis in these resistant cancer cell lines and highlights the close relationship with BAX activation in these solid tumor cancer cell lines.

To further identify survival mechanisms adopted by cancer cell lines in order to avoid apoptosis, we conducted BH3-profiling methodology, an alternative approach to identify survival mechanisms adopted by cancer cell lines to avoid apoptosis (FIG. 1G and FIG. 13A). BH3-profiling analysis demonstrated that cell lines rely on: 1) being “unprimed” to apoptosis; depolarization did not occur upon treatment with sensitizer BH3 peptides e.g. BAD, HRK,

NOXA, but only occurred upon addition of activator BH3-only peptides, e.g. BIM, BID, PUMA, consistent with BAX/BAK not being activated at basal conditions, or 2) dependent on one or more anti-apoptotic BCL-2 proteins for survival; depolarization occurred upon adding a specific sensitizer BH3-only peptides and not only upon addition of an activator BH3 peptide (FIG. 1G, FIGS. 13A-C). Interestingly, most BTSA1.2 resistant and Navitoclax resistant cell lines were categorized into two major anti-apoptotic survival mechanisms: anti-apoptotic BCL-XL dependent or “unprimed” to apoptosis (FIG. 1H-I, FIGS. 13A-C). Indeed, the BH3-profiling data do not support that the majority of solid tumor cell lines are BCL-XL dependent for their survival since similar depolarization from HRK and BAD peptides is observed only with a few cell lines. The BH3-profiling data do not exclude the case that activated BAX by BTSA1.2 or BIM BH3 peptide can be still controlled by the availability of BCL-XL to neutralize activated BAX. Therefore, since some anti-apoptotic BCL-XL-dependent cell lines were resistant to inhibition of BCL-XL by Navitoclax (FIGS. 1D, 1H) this suggested to that another pro-survival mechanism, as “unprimed” to apoptosis, could play a role in apoptosis resistance in these cell lines as well. Indeed, by examining the apoptotic priming status of solid tumor and hematological malignancies with activator BIM BH3 peptide, we determined that solid tumors classified as BCL-XL dependent were less primed than hematological malignancies (FIG. 1J). Furthermore, taking into account cell lines that were resistant to both single treatments of BTSA1.2 and Navitoclax, we found that most of these cell lines were unprimed for apoptosis (FIG. 1K).

Collectively, these data indicated that targeting one survival mechanism is not sufficient to induce potent apoptosis in resistant solid tumor cell lines. Therefore, we rationalized that a dual treatment of BTSA1.2 and Navitoclax could overcome both survival mechanisms by enhancing apoptotic priming with direct BAX activation and inhibiting anti-apoptotic blockade to promote apoptosis (FIG. 1K).

### BTSA1.2 AND NAVITOCLAX SYNERGIZE TO INDUCE APOPTOSIS IN RESISTANT TUMOR CELL LINES

We conducted a screen in our panel of cancer cell lines to compare the cytotoxic activity of Navitoclax with the cytotoxic activity of Navitoclax in combination with a fix sensitizing concentration of BTSA1.2 (loss of cell viability <20%). The combination treatment of Navitoclax with a fixed sublethal dose of BTSA1.2 increased cytotoxicity in many cancer cell lines including resistant solid tumors such as pancreatic and colorectal carcinomas regardless of common genetic alterations (e.g., TP53, RAS) (FIGS. 2A-2C, FIG. 12A). Based on the fold change (FC) of the IC₅₀ in cell viability upon drug treatments, cell lines were categorized as sensitive to the combination (IC₅₀ fold change >5×), having intermediate sensitivity to the combination (ICso fold change 2-4×), or resistant to the combination (IC₂₀ fold change <2×) (FIGS. 2A-2C, FIG. 12A). Cancer cell lines sensitive to the combination were predicted to have a synergistic effect upon the dual treatment. Indeed, in cell lines from different tumor types, upon dual treatment cell viability was synergistically decreased across different concentrations (FIGS. 2D-2E, FIG. 12B-12C). Consistent with synergistic efficacy on apoptosis induction, a significant increase of caspase 3/7 activation was observed upon dual treatment compared to the activity of single agents (FIG. 2F). Importantly, the synergistic effect in loss of viability and induction of apoptosis was determined to be BAX-dependent, as Calu-6 cells that are sensitive to the BTSA1.2 and Navitoclax combination (BNc), become resistant to the combination when these cells lack BAX expression. (FIGS. 2G-2H) However, Calu-6 BAX KO cells are still sensitive to the generic apoptosis inducer staurosporine presumably through BAK-mediated apoptosis (FIGS. 2I, 2J). Thus, the BNc seems a promising therapeutic strategy as these compounds synergize to promote apoptosis in solid tumors and hematological malignancies regardless of the mutational background.

To further dissect the contribution of BCL-XL or BCL-2 inhibition, since Navitoclax is able to inhibit both proteins in cells, we investigated both selective BCL-XL inhibitor A-1331852 and selective BCL-2 inhibitor Venetoclax in combination with the BAX activator BTSA1.2. We evaluated a set of cell lines from solid tumors e.g. SW480, COLO230 and hematological malignancies OCI-AML3, U937 in which expression for either BCL-XL or BCL-2 or both proteins can be detected (FIG. 10A). In SW480 and COL0230 cell lines, BCL-2 protein expression is not detected by western blot and BCL-XL is well expressed, Venetoclax is not effective at submicromolar concentrations and synergy is not observed with Venetoclax and BTSA1.2 combination (FIG. 26A, 26B). In contrast, BCL-XL specific inhibitor A-1331852 is potent in SW480 cell line and shows strong synergy with BTSA1.2 in SW480 (FIG. 26A). A-1331852 is not effective in COL0230 presumably due to MCL-1 (FIG. 10A) but it demonstrated synergy with BTSA1.2 (FIG. 26B). Furthermore, in OCI-AML3 and U937 cells where BCL-2 is expressed, Venetoclax is effective as single agent and demonstrated synergy when combined with BTSA1.2 (FIG. 26C, 26D). More specifically, Venetoclax is more effective and synergistic with BTSA1.2 in OCI-AML3 cells than in U937 cells, most likely because OCI-AML3 is more dependent on BCL-2 protein and has higher BCL-2 protein levels (FIG. 10A). A-1331852 is moderately potent as single agent in OCI-AML3 and U937 cells but it demonstrates also synergy with the BTSA1.2 combination (FIGS. 26C, 26D). These data support that BCL-XL is the anti-apoptotic protein that controls BAX in majority of resistant solid tumor cell lines as BCL-2 is not detected or BCL-2 inhibition has limited effect in these cell lines.

### BAX INTERACTION WITH BCL-XL DICTATES SENSITIVITY TO BTSA1.2 AND NAVITOCLAX COMBINATION

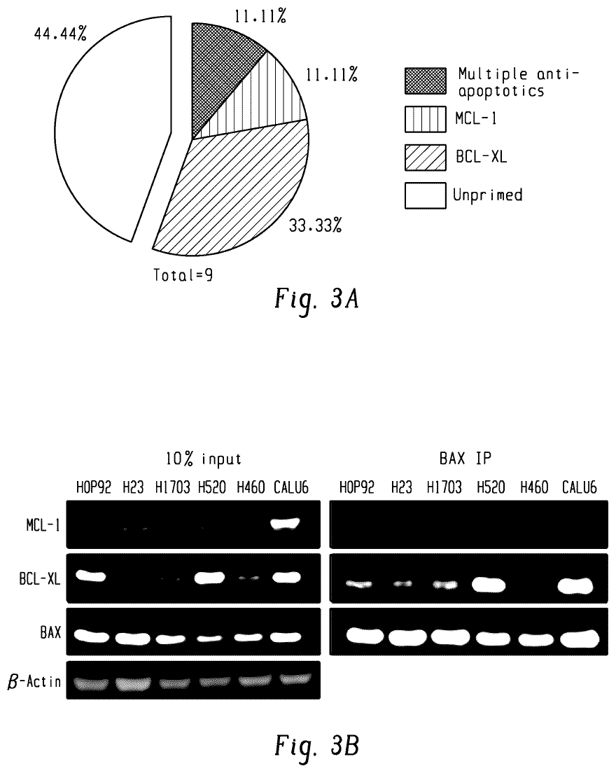

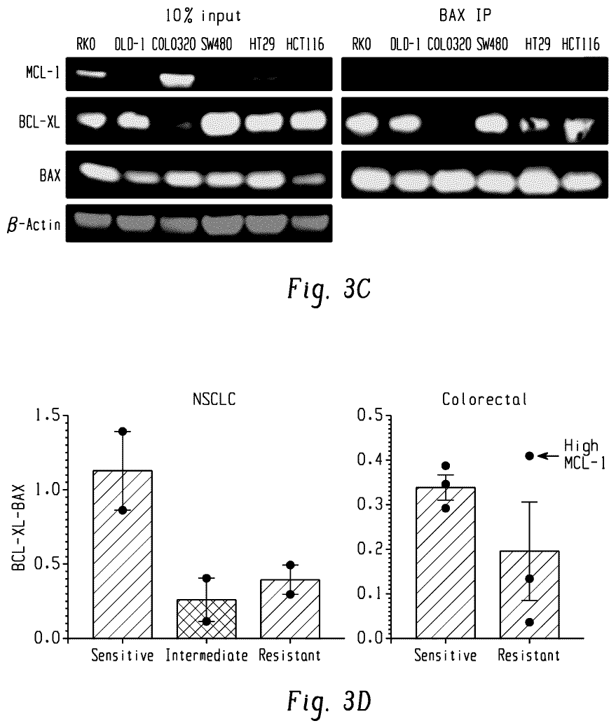

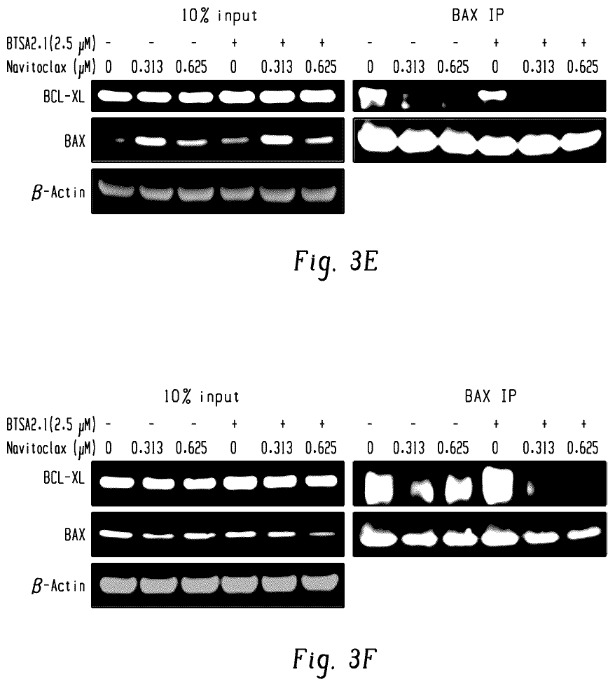

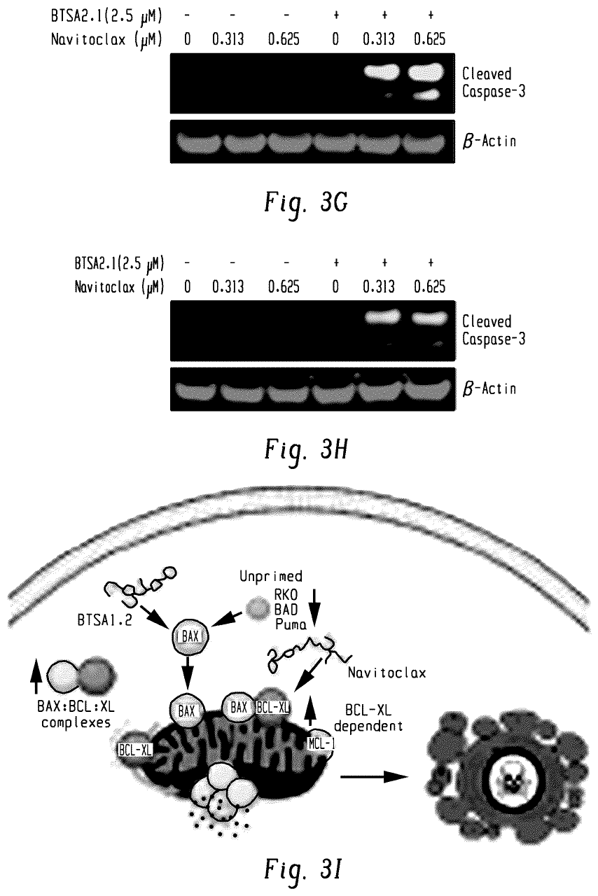

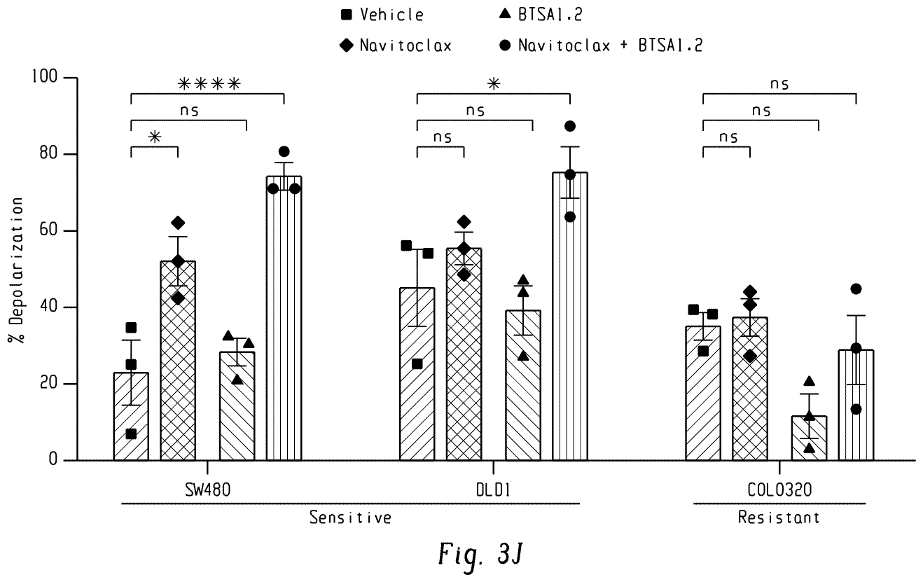

To identify determinants of sensitivity for the BNc, we looked at the BCL-2 family protein expression and interactions. combination of BTSA1.2 and Navitoclax, the protein expression levels of BCL-2 family members was examined BH3-profiling indicated that cell lines sensitive to the combination were categorized as anti-apoptotic BCL-XL dependent or unprimed to apoptosis (FIG. 3A). Interestingly, these survival mechanisms were adopted by resistant cell lines to either single agent treatment of Navitoclax or BTSA1.2 (FIG. 1H, II), indicating that the dual targeting of BAX and BCL-XL was able to overcome these two-survival mechanisms in previously categorized resistant cells to single agent treatment.

We next evaluated how the BCL-2 family is modulated in sensitive cells to the BNc. When we examined protein expression levels of BCL-2 family members, only BAX:BCL-XL levels marginally correlated with the sensitivity towards the combination (FIG. 26E). This finding suggested that interactions among BCL-2 family members, and not the protein levels, could be regulating sensitivity towards the combination. To dissect this, we co-immunoprecipitated BAX and interrogated its binding with BCL-XL and MCL-1 anti-apoptotic proteins in several colorectal and non-small cell lung cancer cell lines (FIG. 3B, 3C, and FIG. 26E). Sensitive cell lines, in general, formed higher levels of BAX:BCL-XL complexes than cell lines resistant to the combination (FIG. 3D). BAX complexes formed are consistent with the finding that BCL-XL is a key player in the apoptotic resistance of these cancer cell lines as immunoprecipitated BAX had more interactions with BCL-XL (FIGS. 1B, 3-3C). Next, we treated sensitive colorectal and non-small cell lung cancer cell lines to the combination. Treatment with Navitoclax disrupted BAX:BCL-XL complexes and upon co-treatment with BTSA1.2, additional complexes were disrupted, hence relieving the suppression of active BAX by BCL-XL (FIGS. 3F,3G). Importantly, only upon the combination treatment with BTSA1.2, additional complexes were disrupted, and apoptosis induction was observed by caspase-3 activation (FIGS. 3E-3H). Consistently, apoptotic priming with activator BIM BH3 peptide was increased upon the combination treatment in sensitive cell lines but not on resistant cells (FIG. 3J). Therefore, our data is consistent with the interaction of BCL-XL with BAX as determinant of the synergistic apoptotic efficacy of the BTSA1.2/Navitoclax combination (BNc) (FIG. 3I).

### COMBINATION OF BTSA1.2 AND NAVITOCLAX IS WELL-TOLERATED IN VIVO

Next, the therapeutic potential of simultaneous targeting the two survival mechanisms with the BTSA1.2 and Navitoclax combination was assessed in vivo. Pharmacokinetics analysis of BTSA1.2 demonstrated favorable properties by oral administration such as substantial half-life (T1/2 ˜15hr) in mouse plasma, excellent oral bioavailability (% F —50%) and significant plasma exposure (AUC ˜100 μM hr) and peak concentration (Cmax ˜8 μM) (FIGS. 16A-16B). We performed a maximum tolerated dose (MTD) study following a standard MTD protocol that included a daily dose of BTSA1.2 with concentrations ranging from 50 mg/kg/po to 300 mg/kg/po for 5 days and monitoring for 14 days. (FIG. 16C). The MTD study indicated that oral administration of BTSA1.2 is safe well tolerated up to 200 mg/kg without dose limiting toxicity (DLT at 300 mg/kg), where BTSA1.2 treated mice showed constant body weights and organs examined were between normal histologic limits (FIGS. 16C-16E). Thus, BTSA1.2 is a BAX activator that can be safely administrated orally and has desirable pharmacokinetics to address therapeutic efficacy.

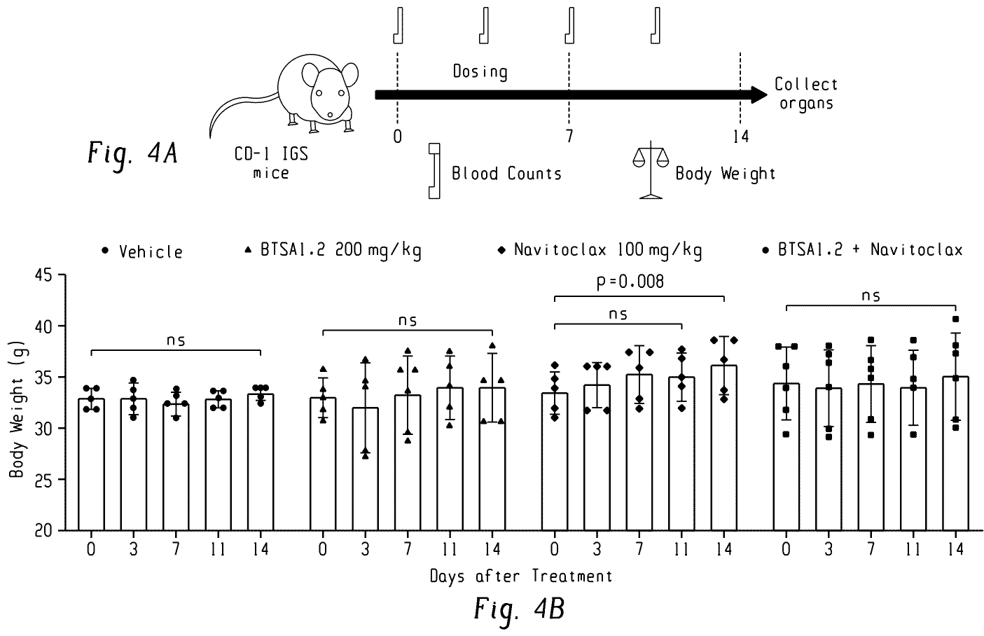

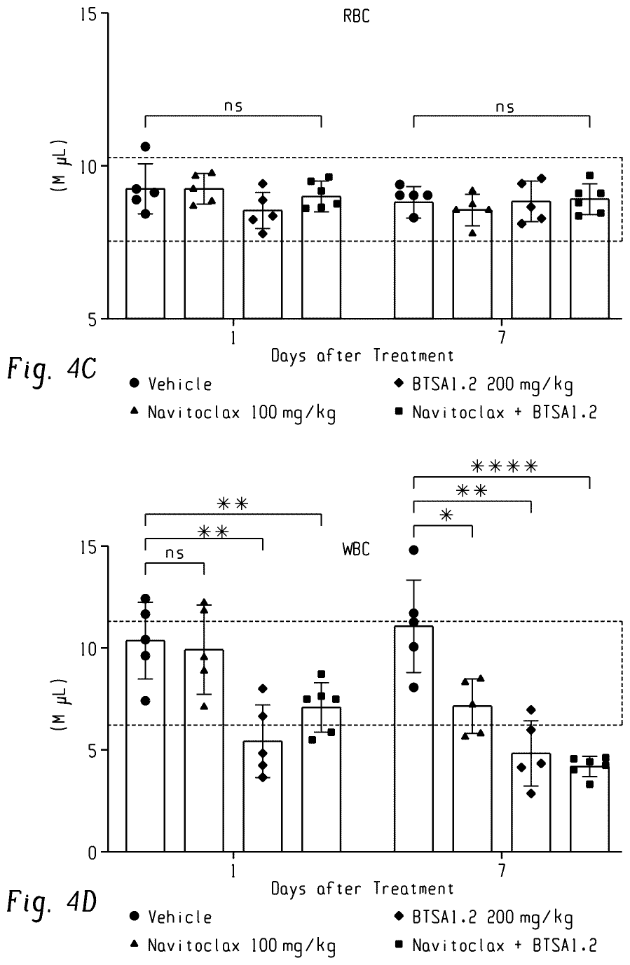

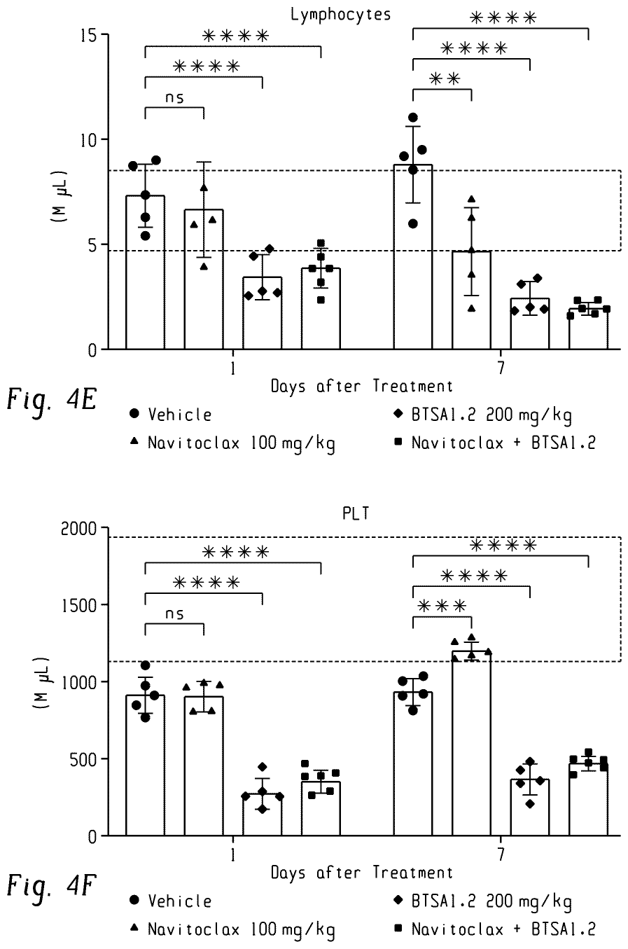

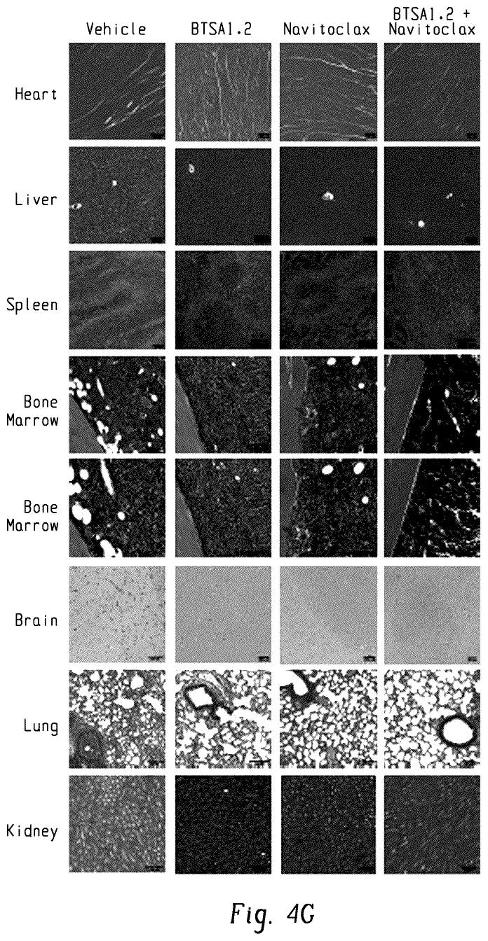

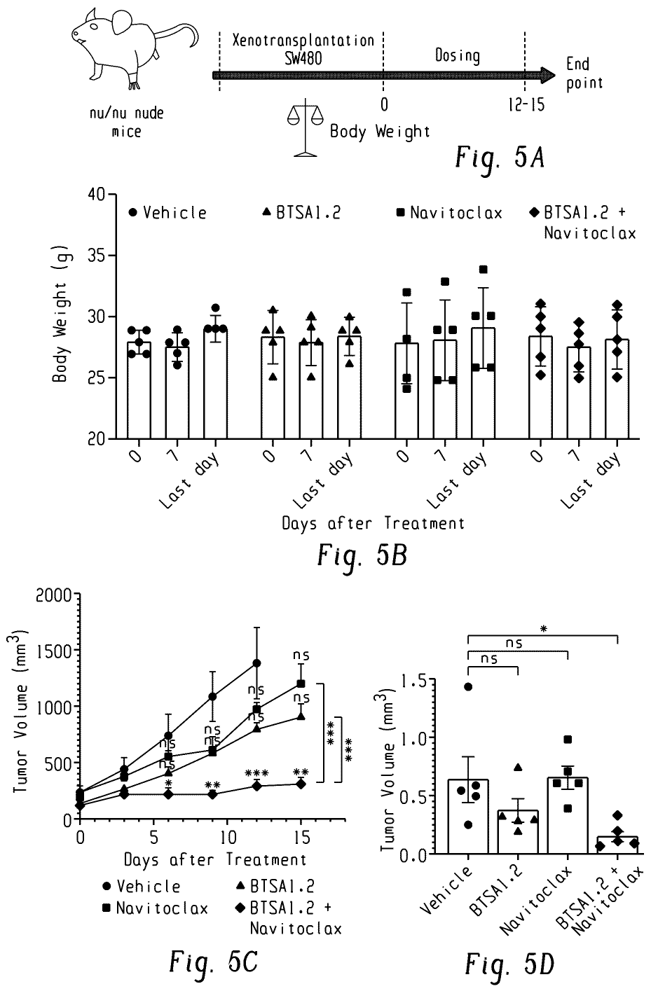

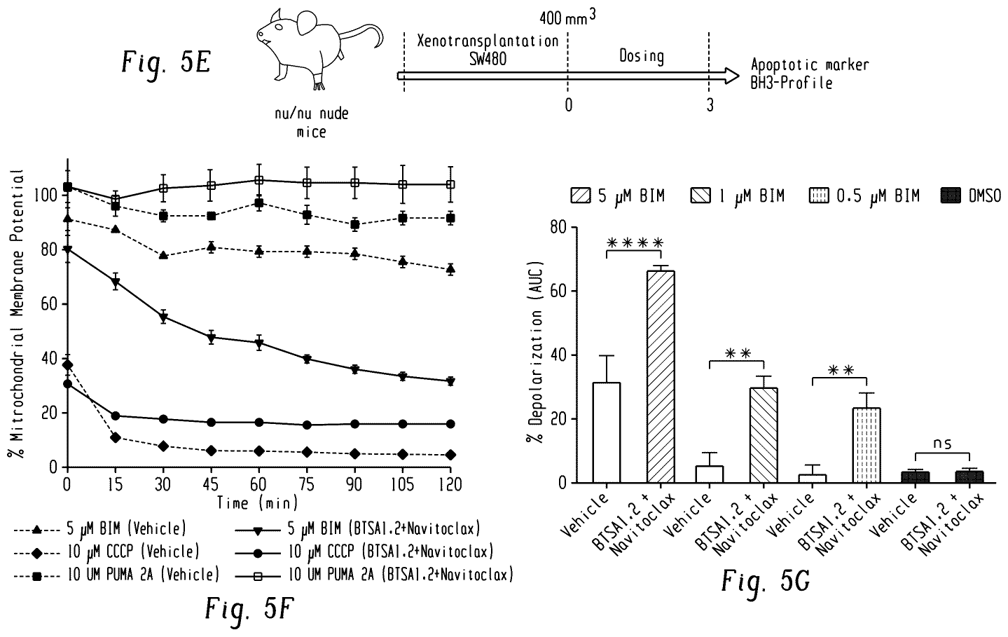

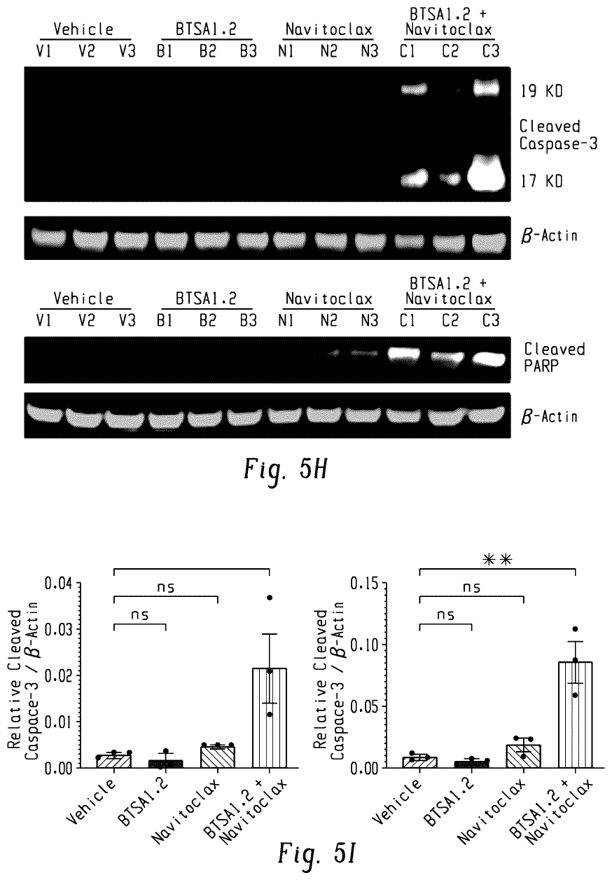

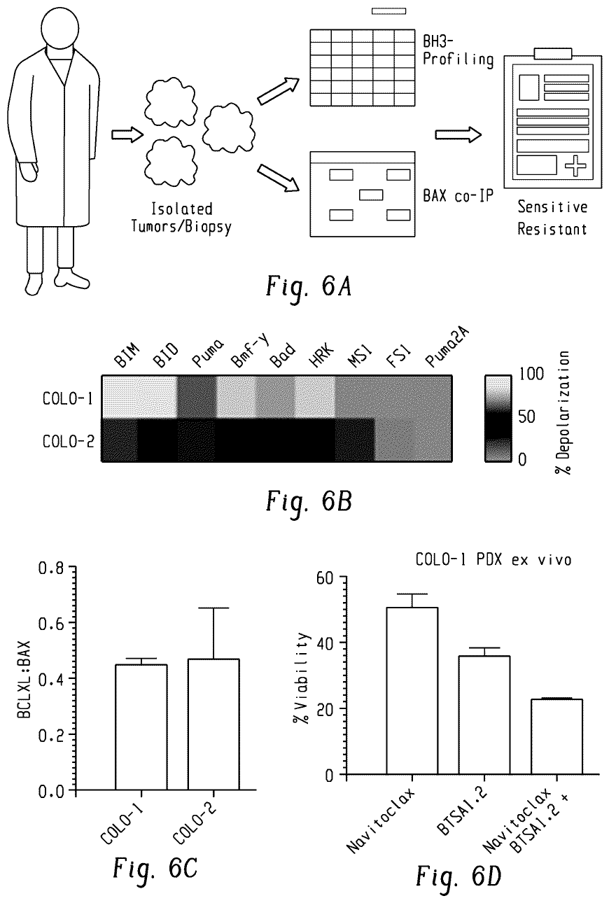

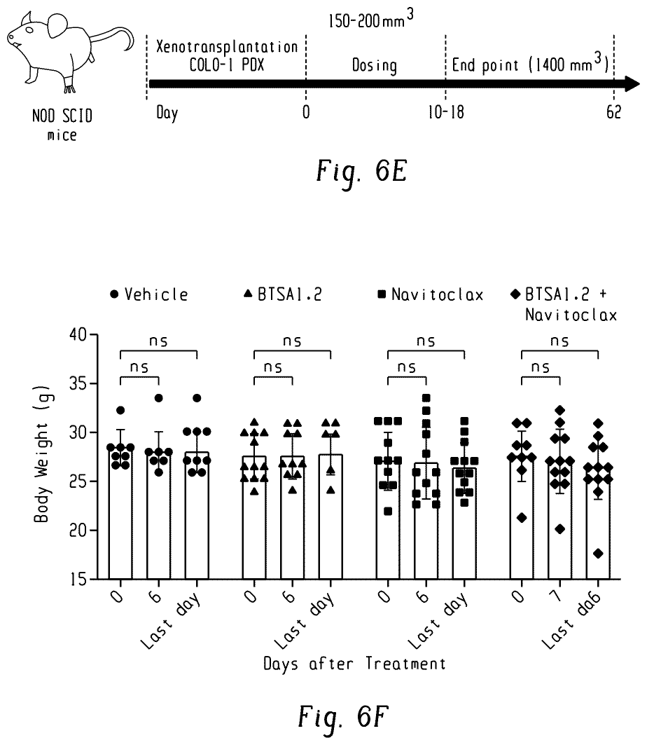

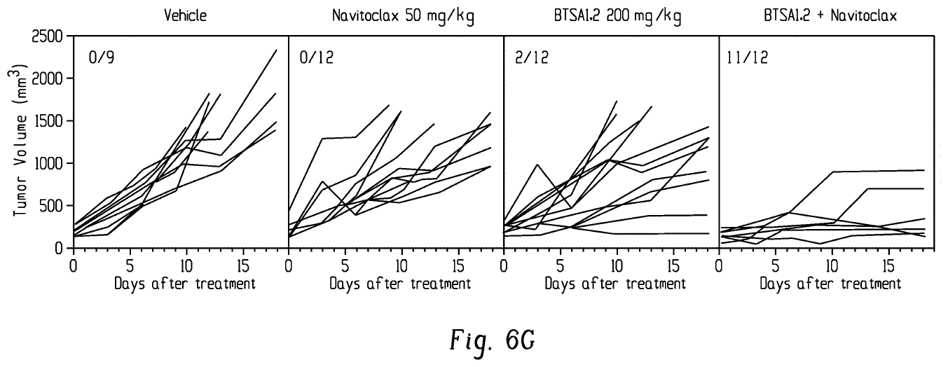

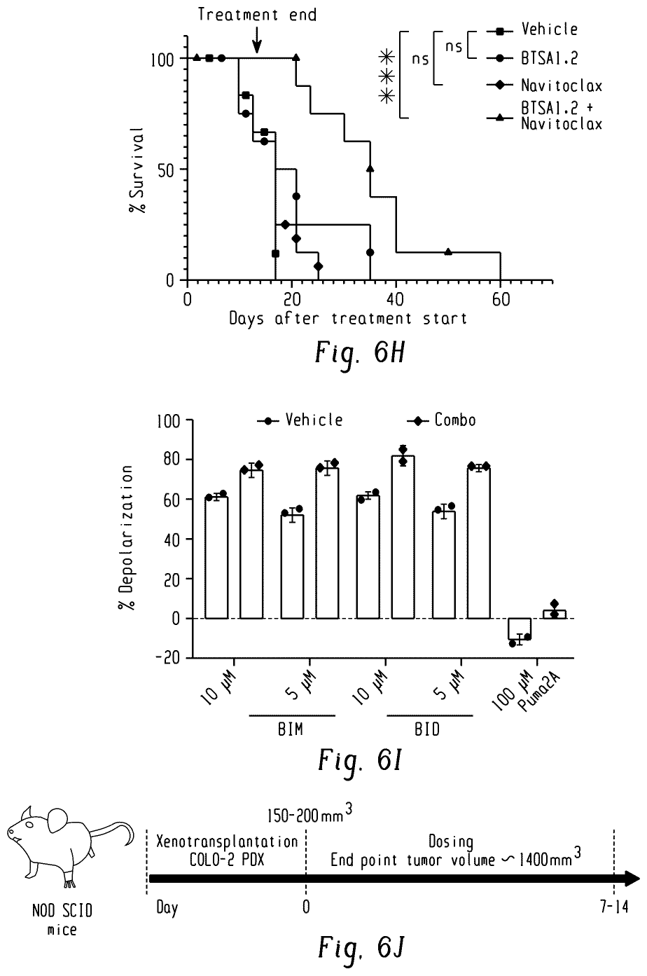

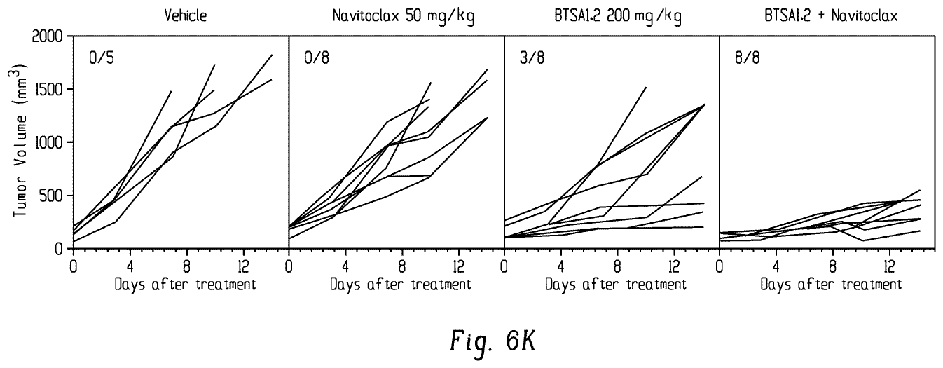

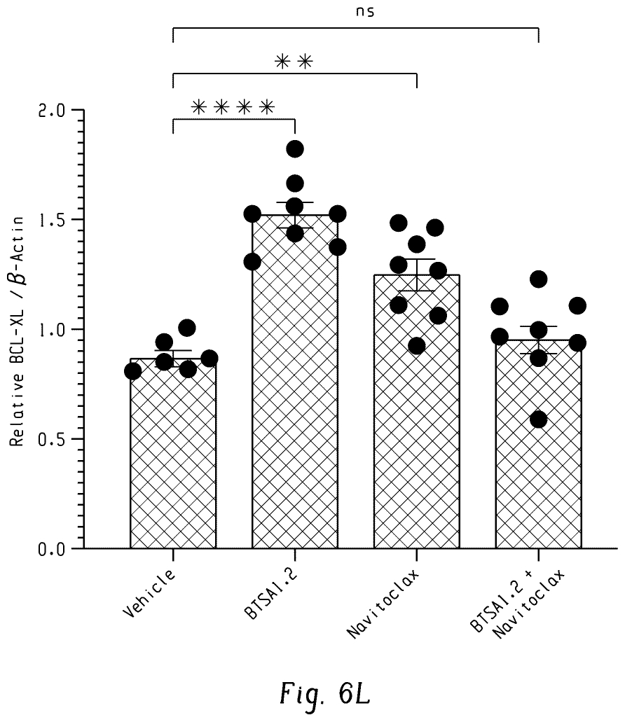

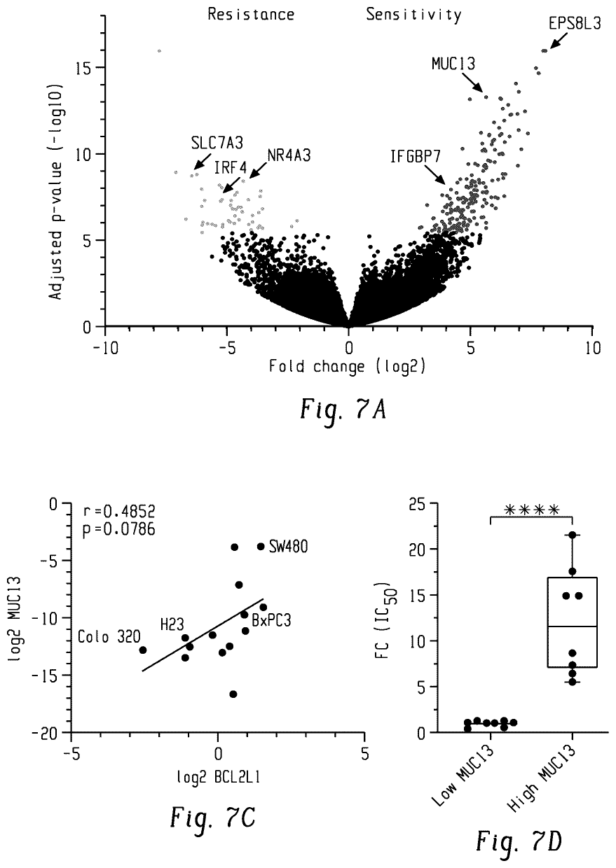

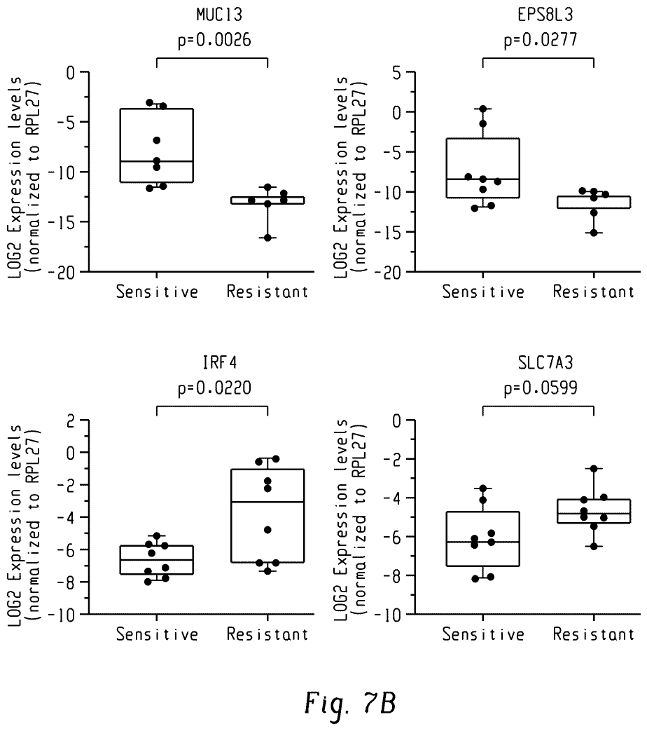

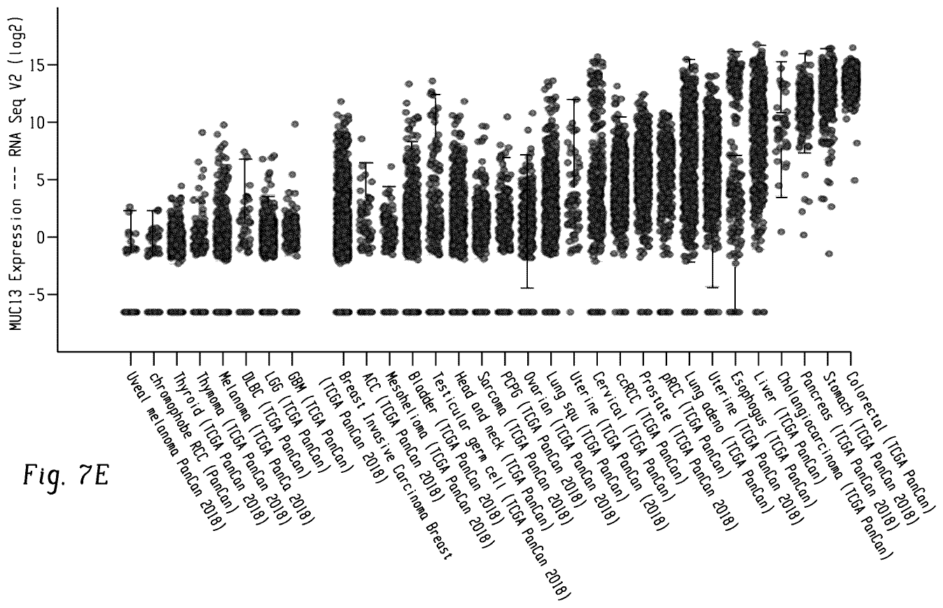

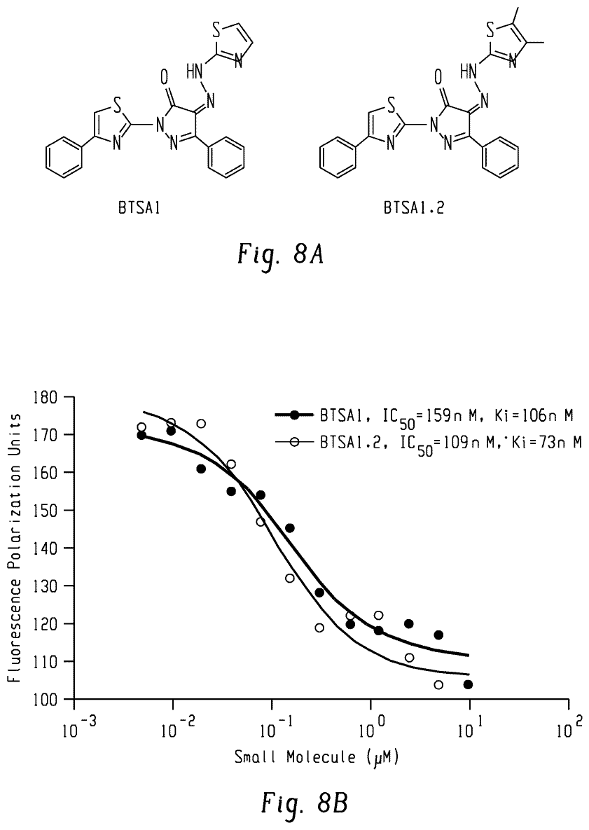

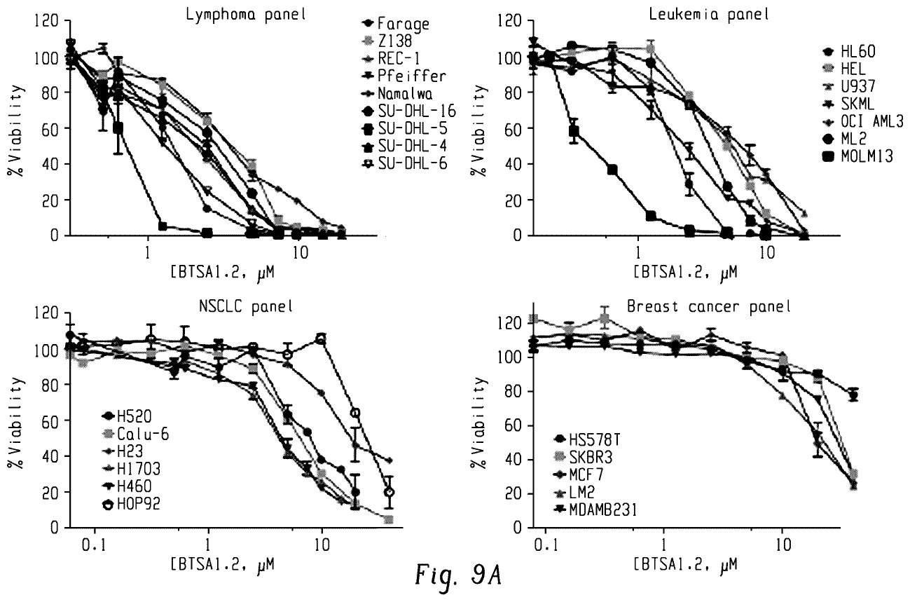

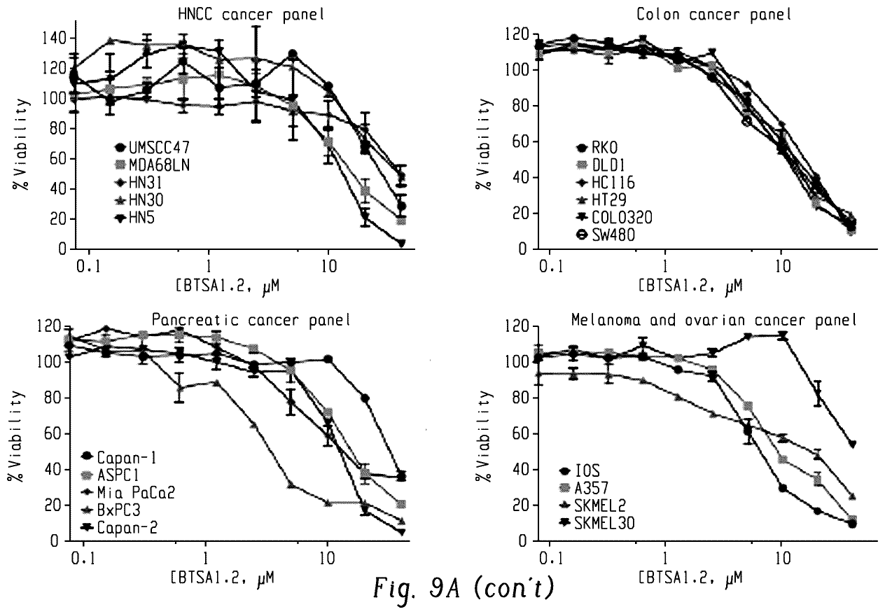

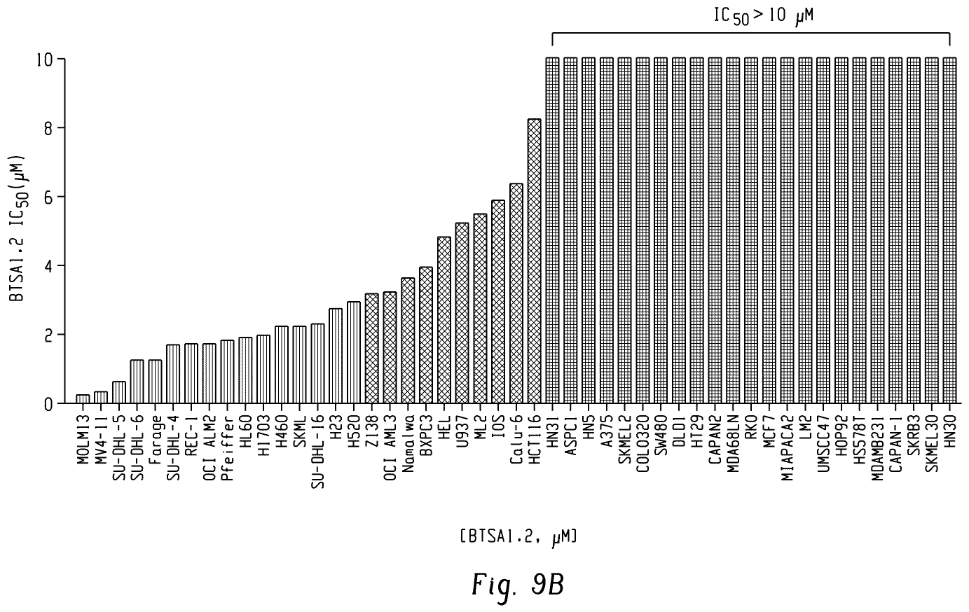

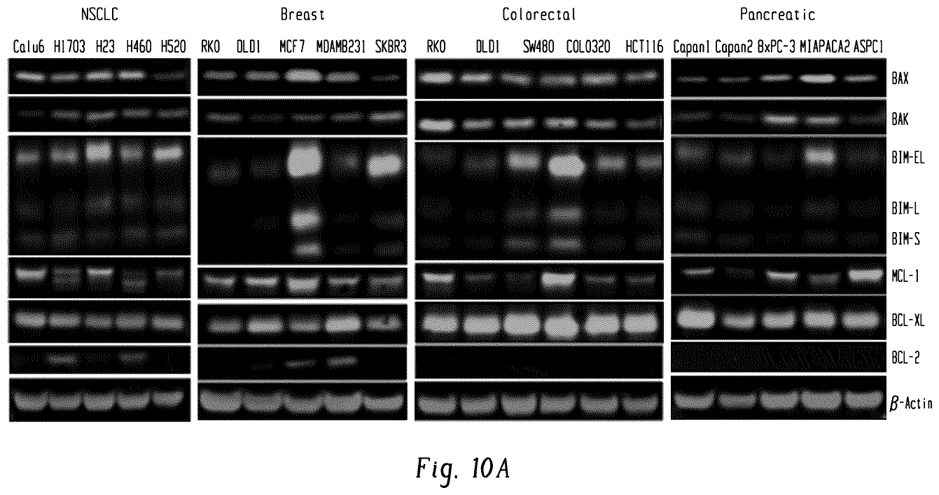

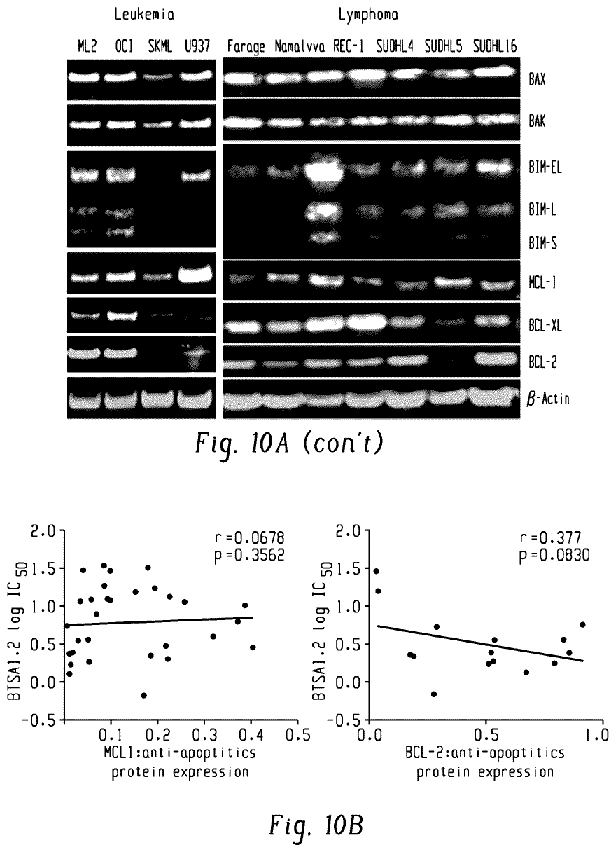

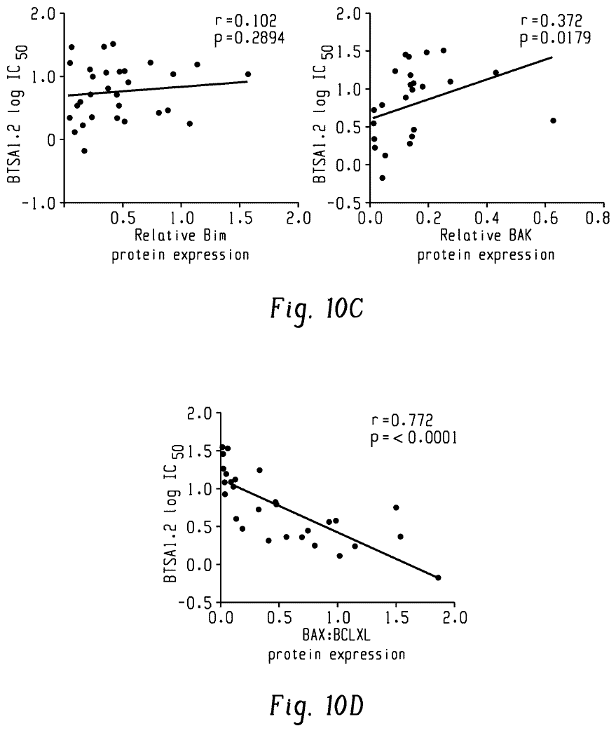

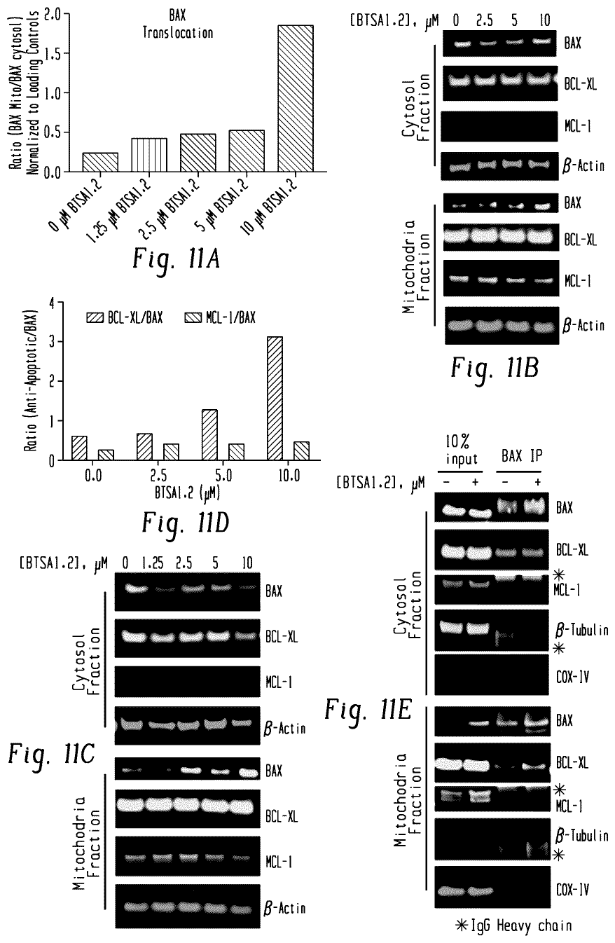

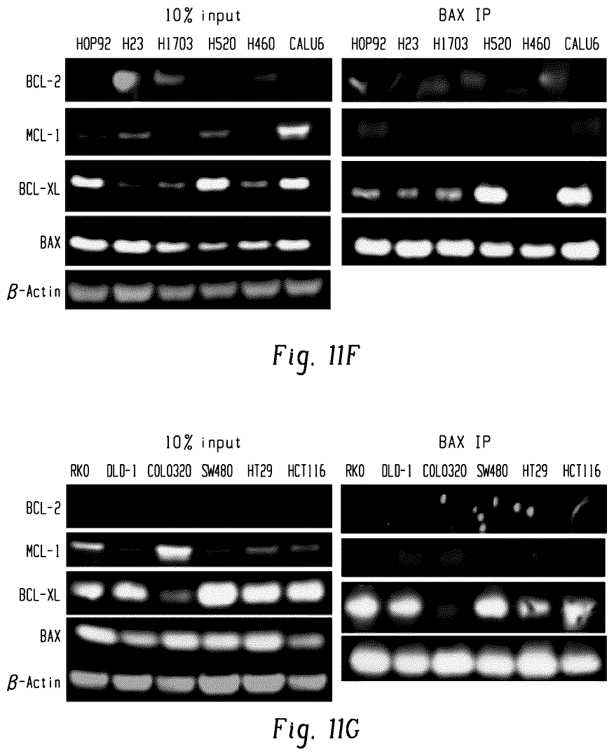

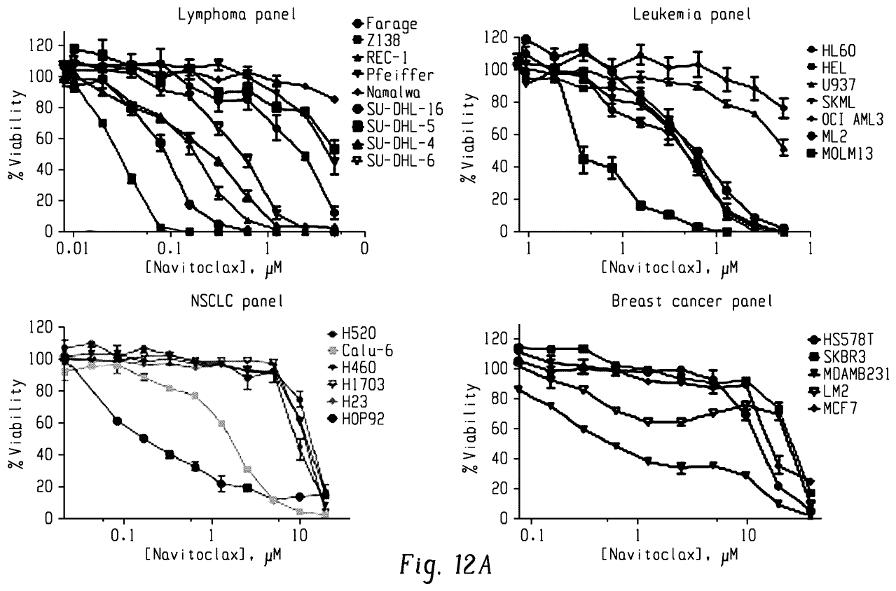

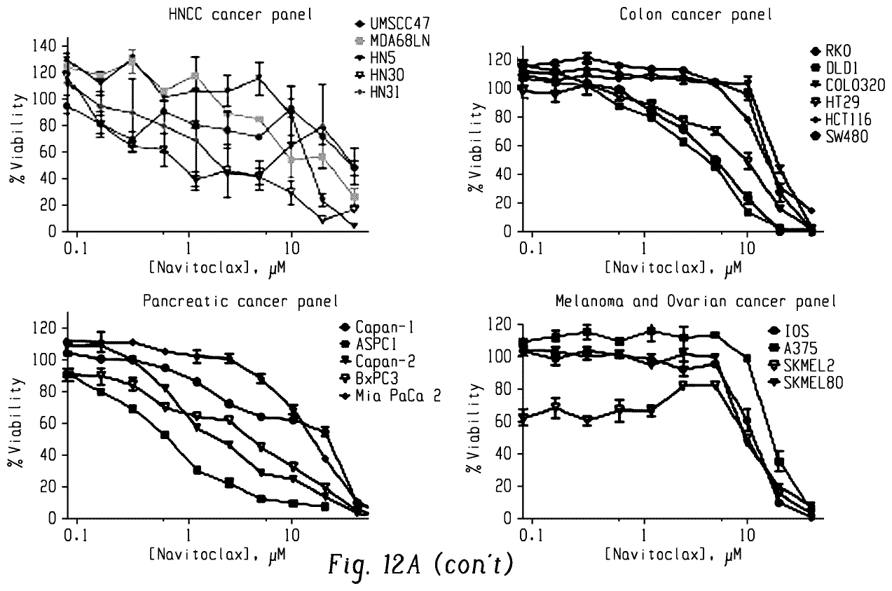

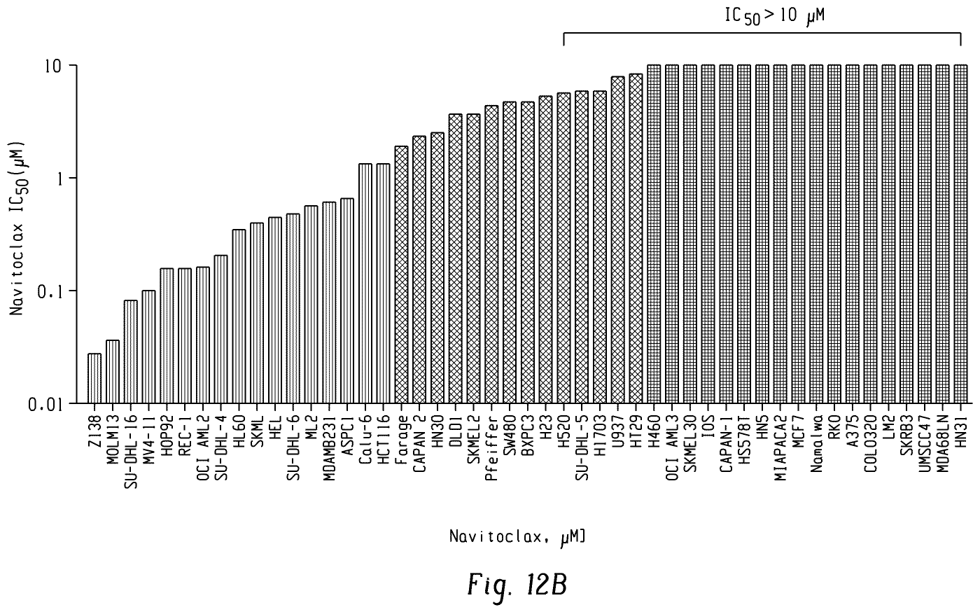

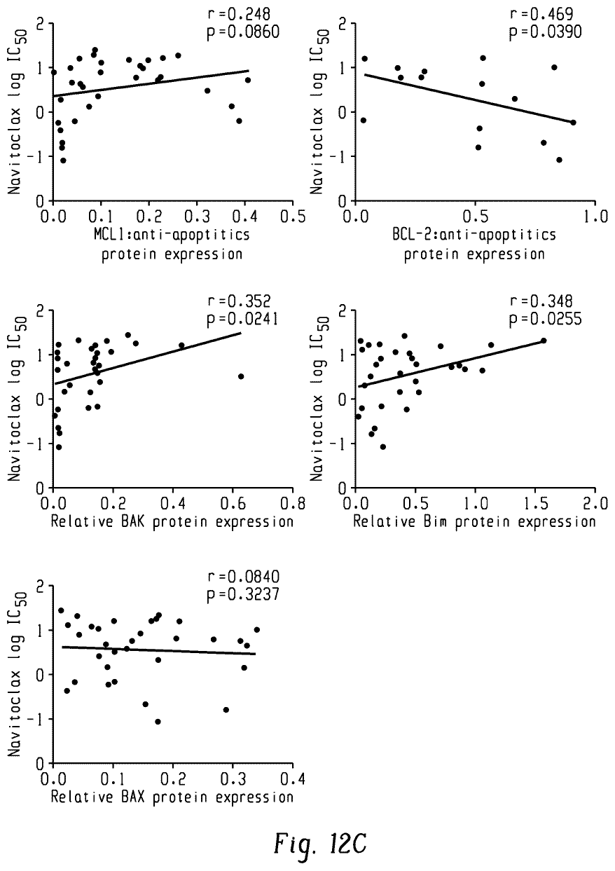

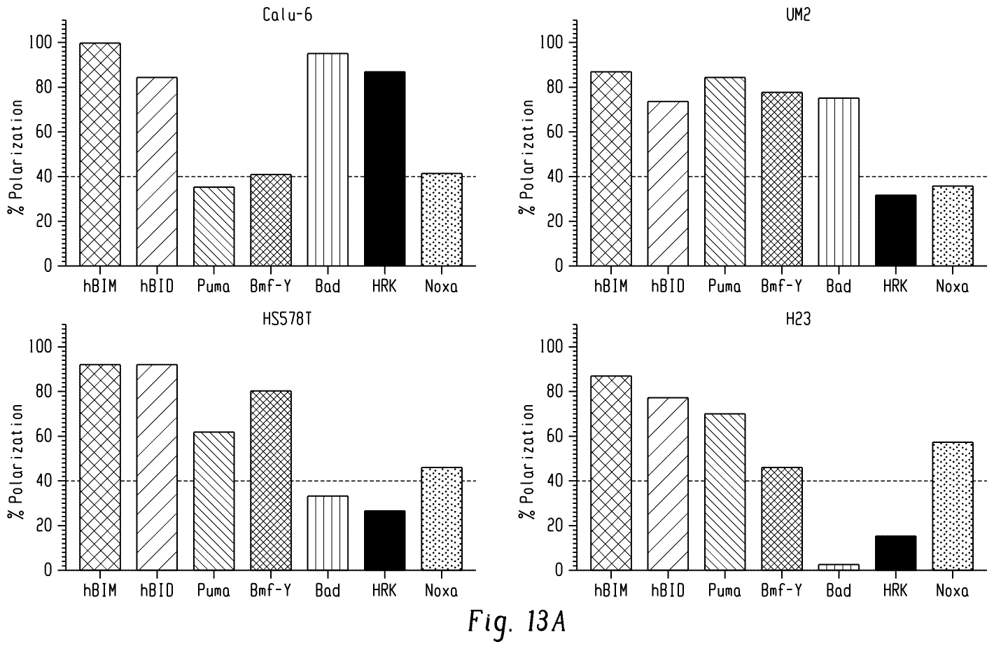

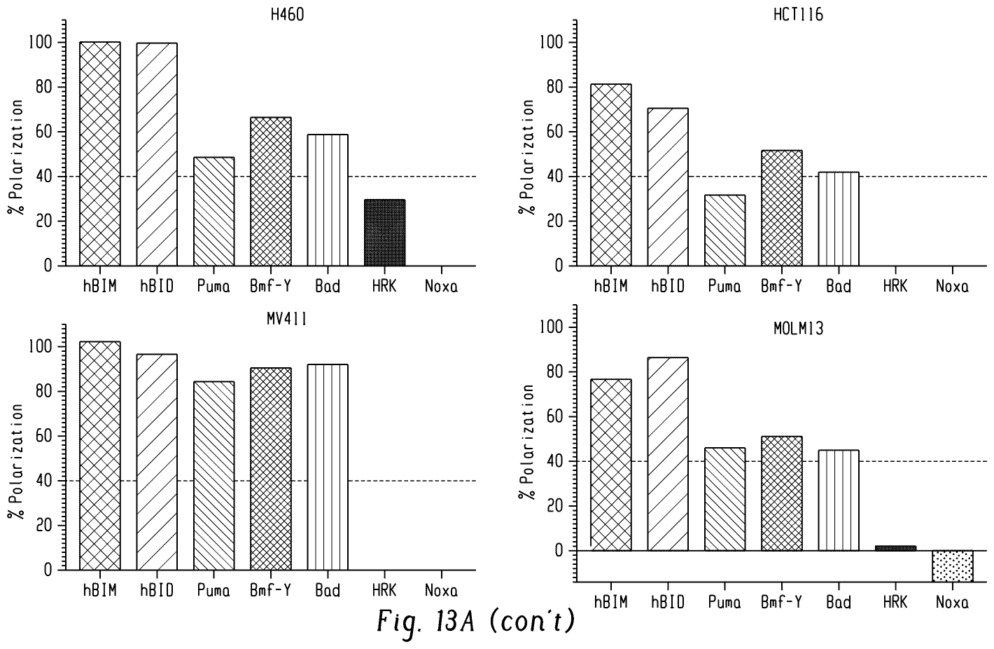

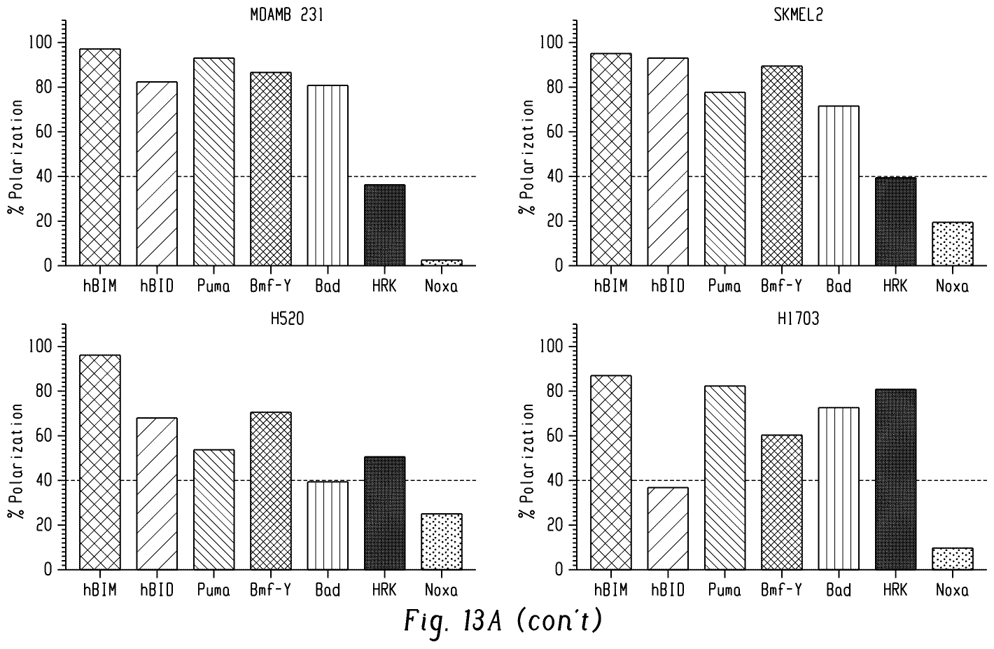

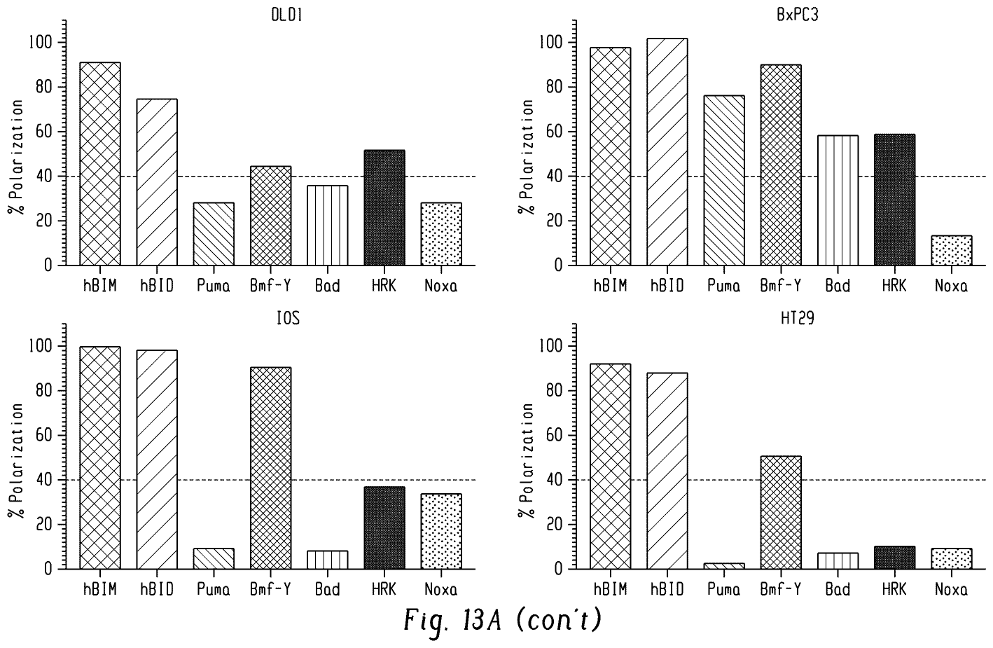

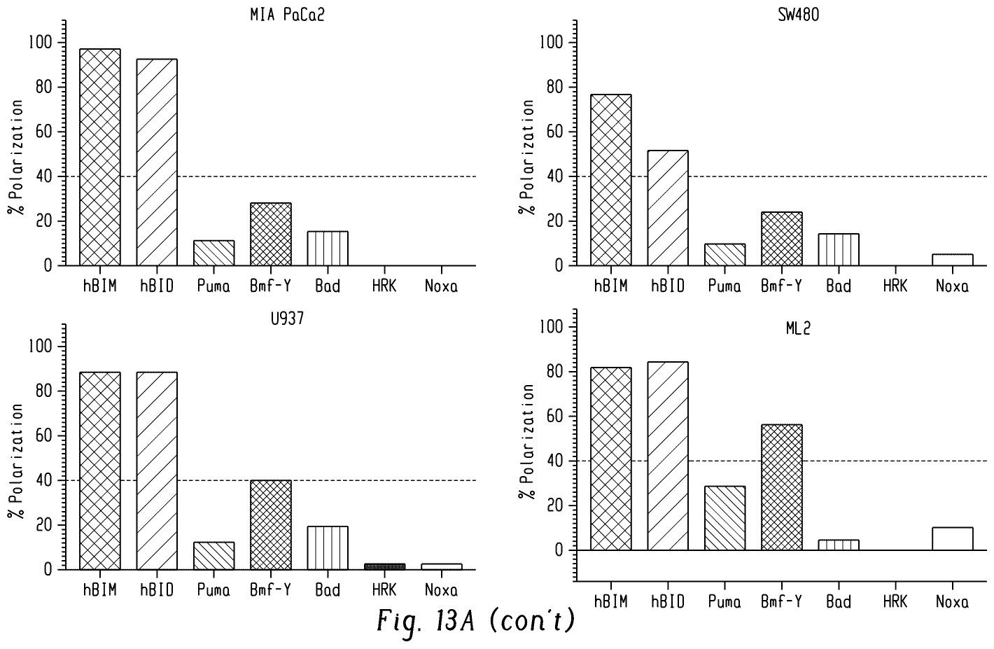

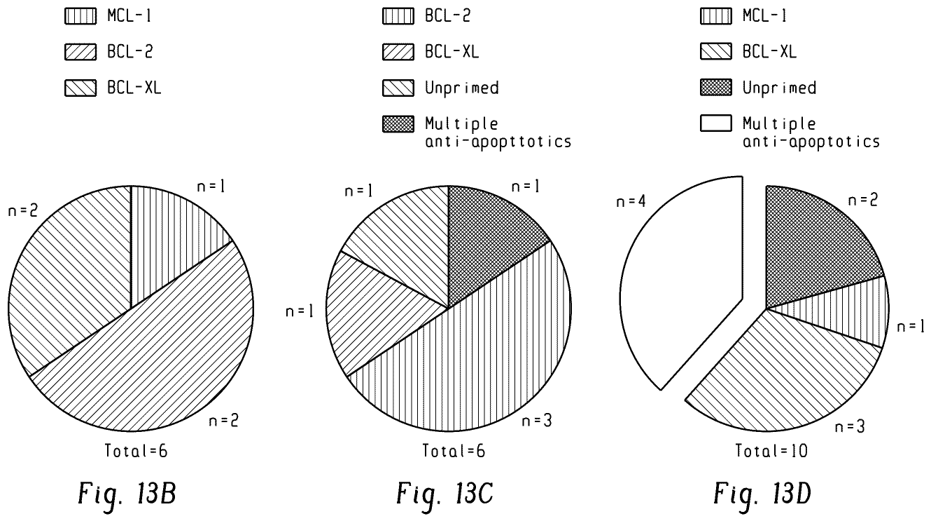

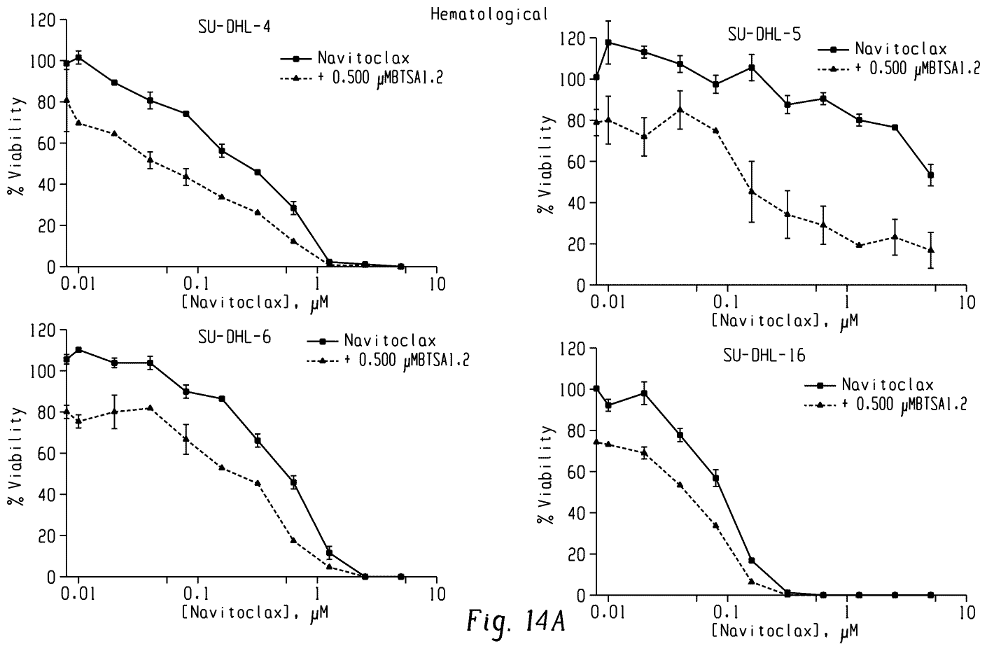

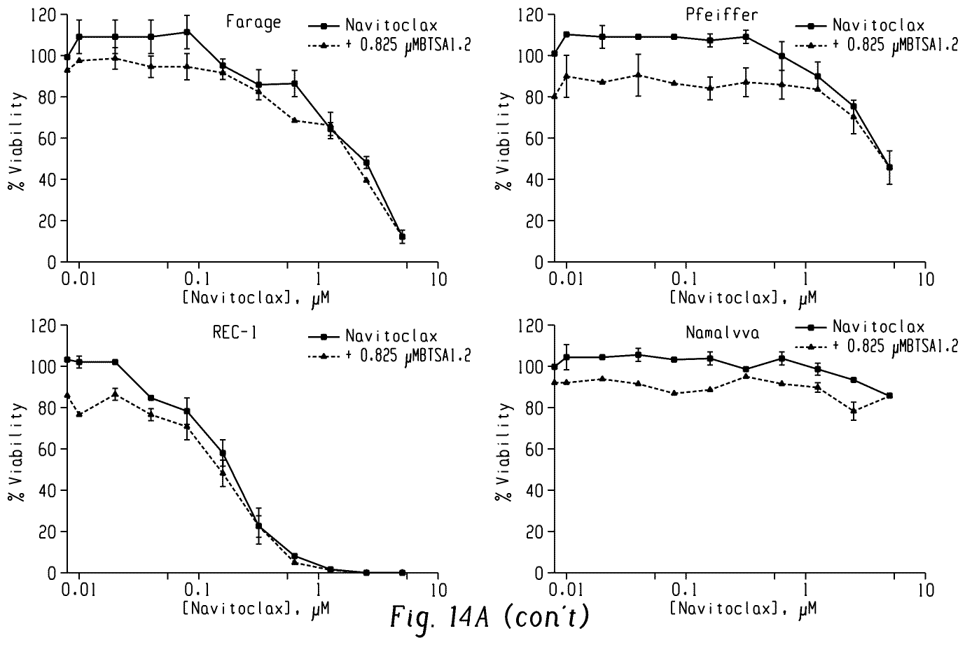

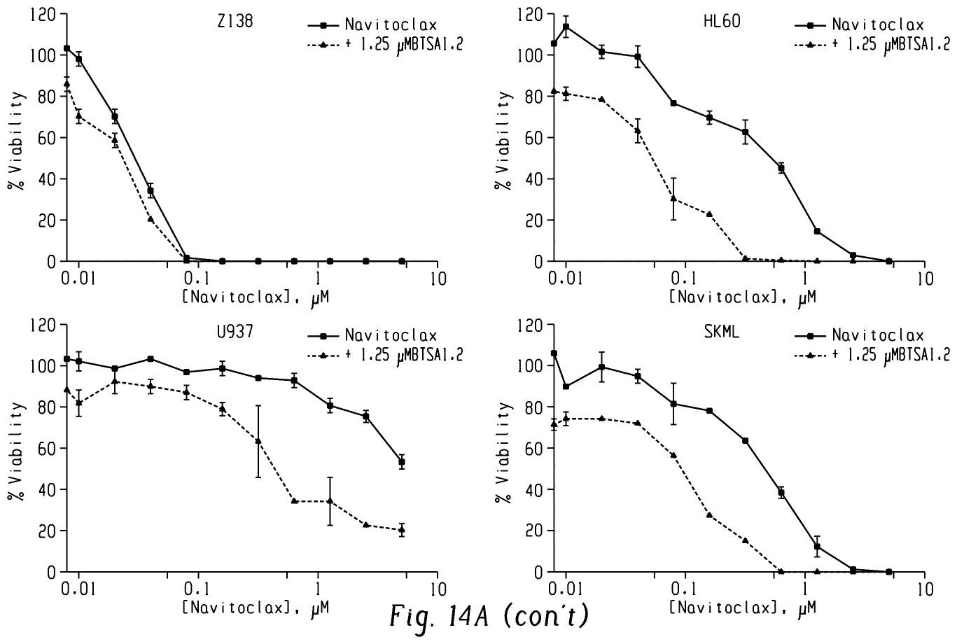

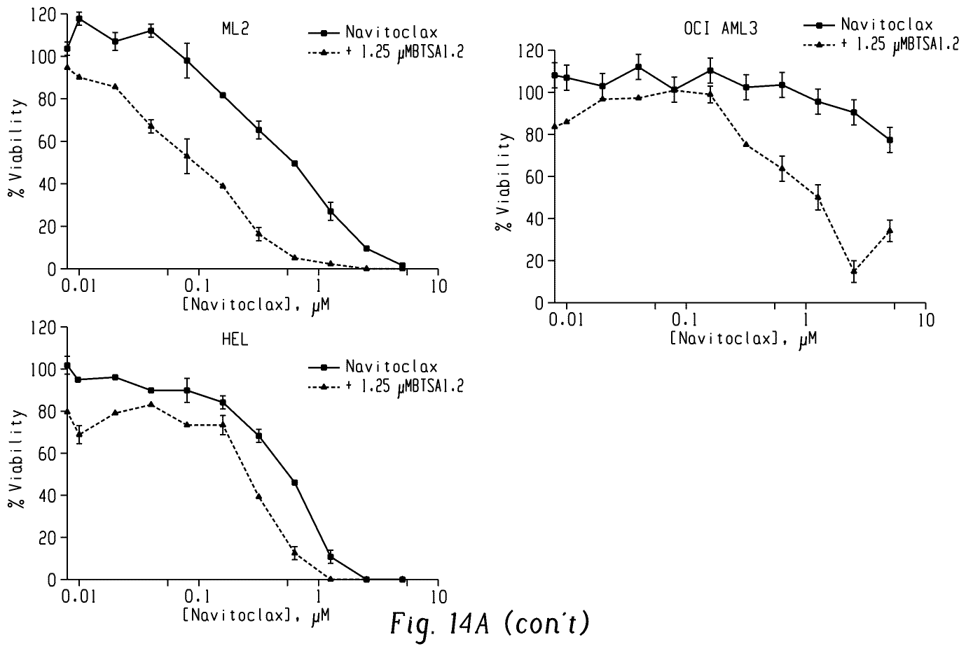

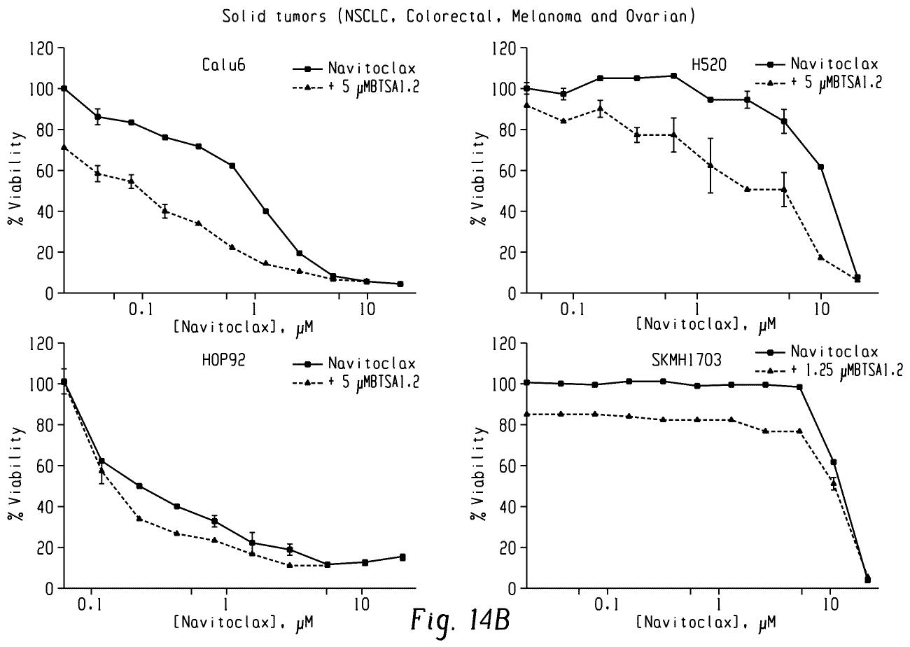

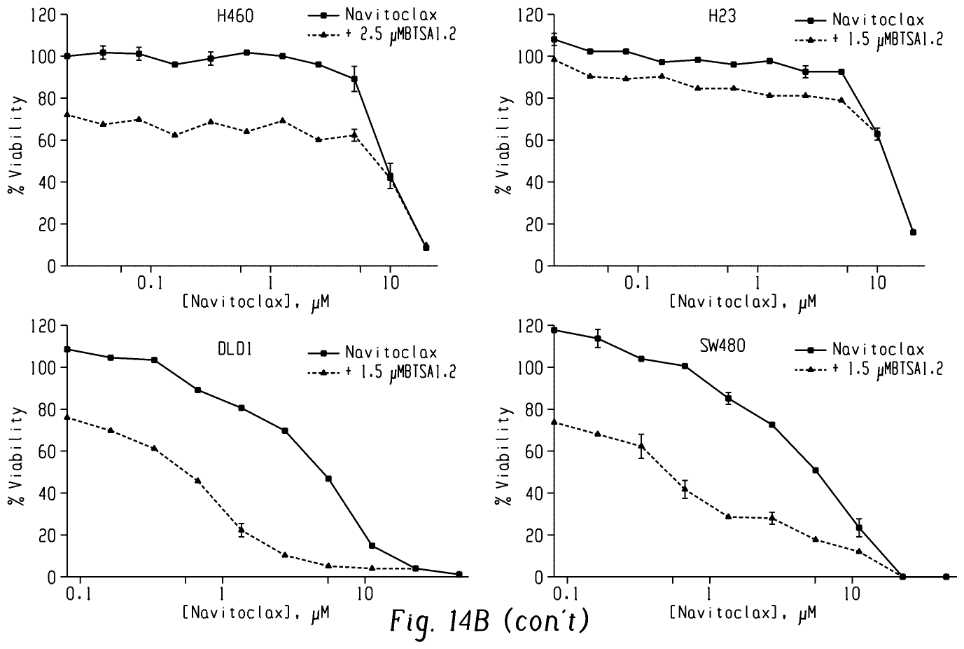

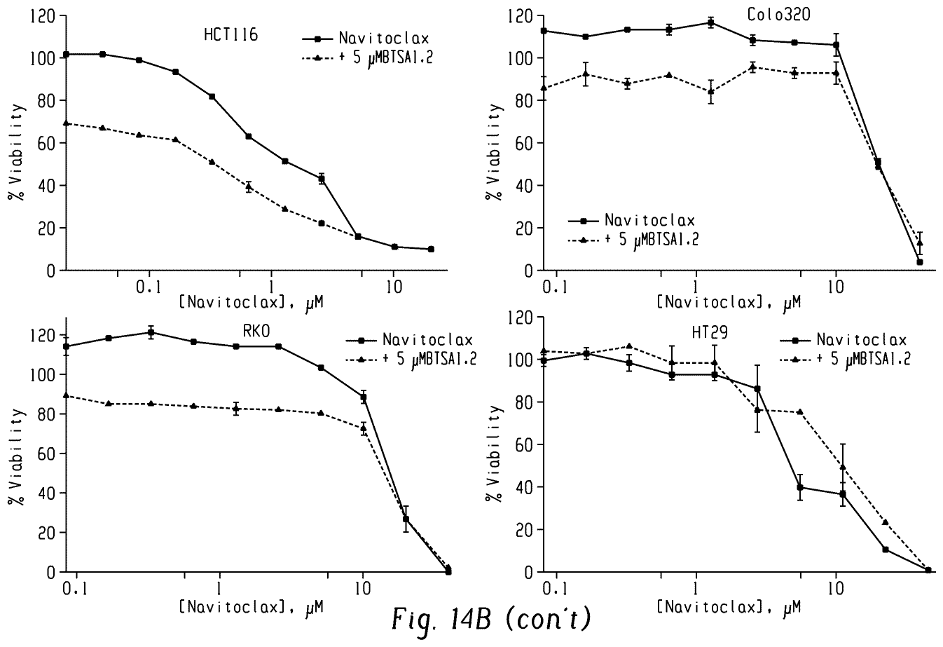

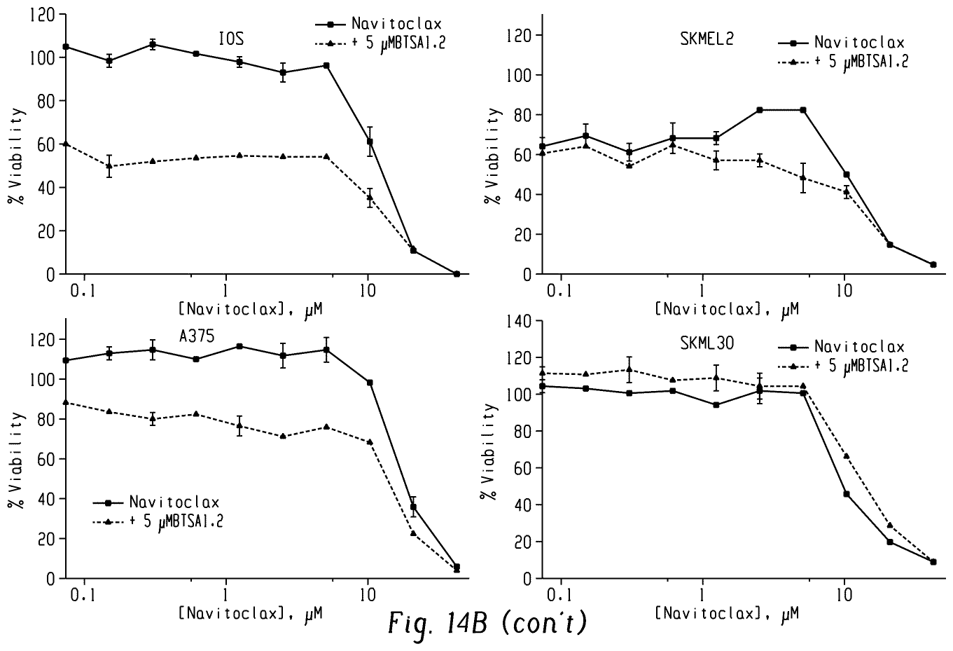

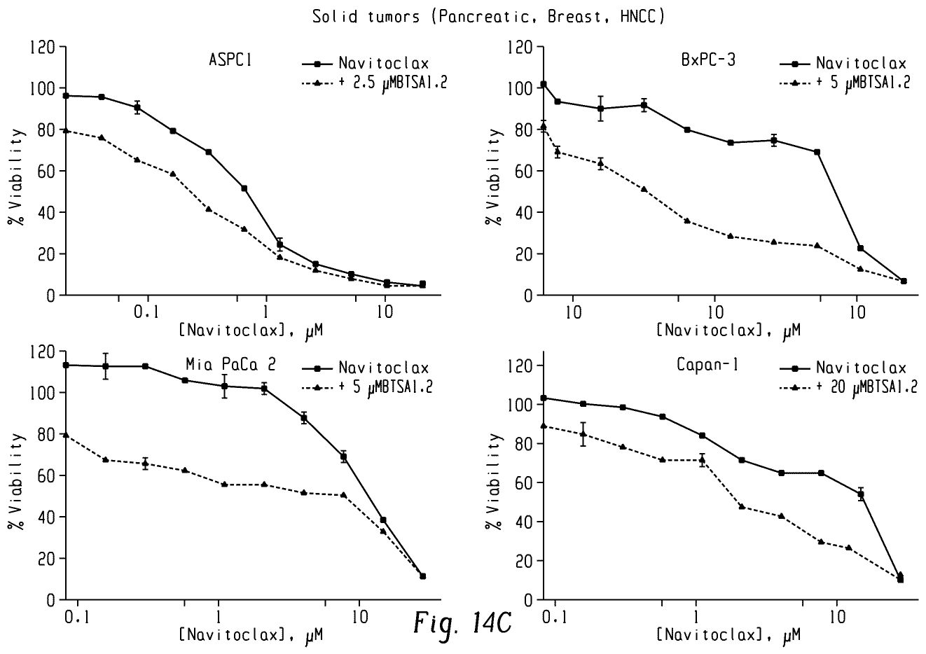

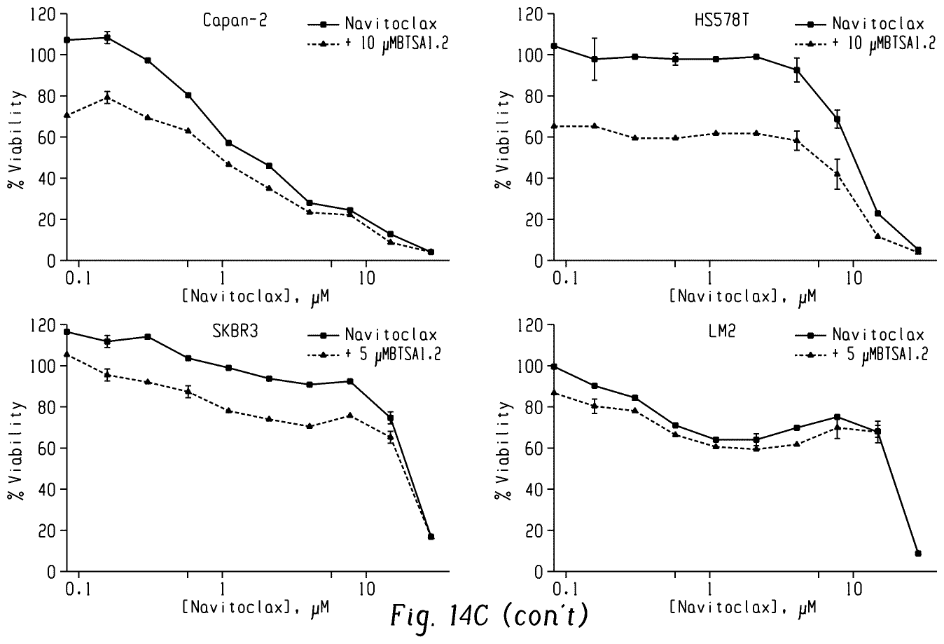

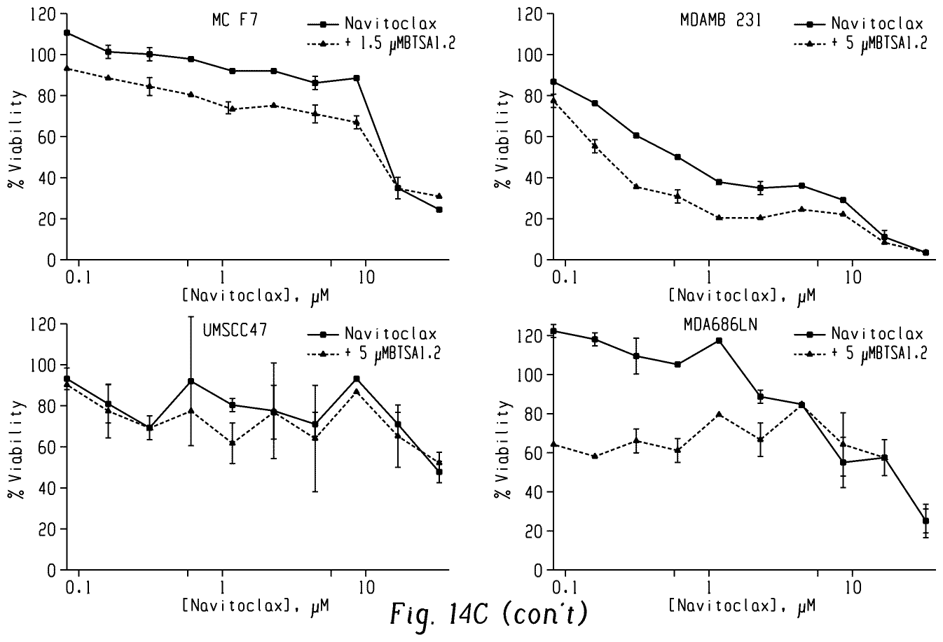

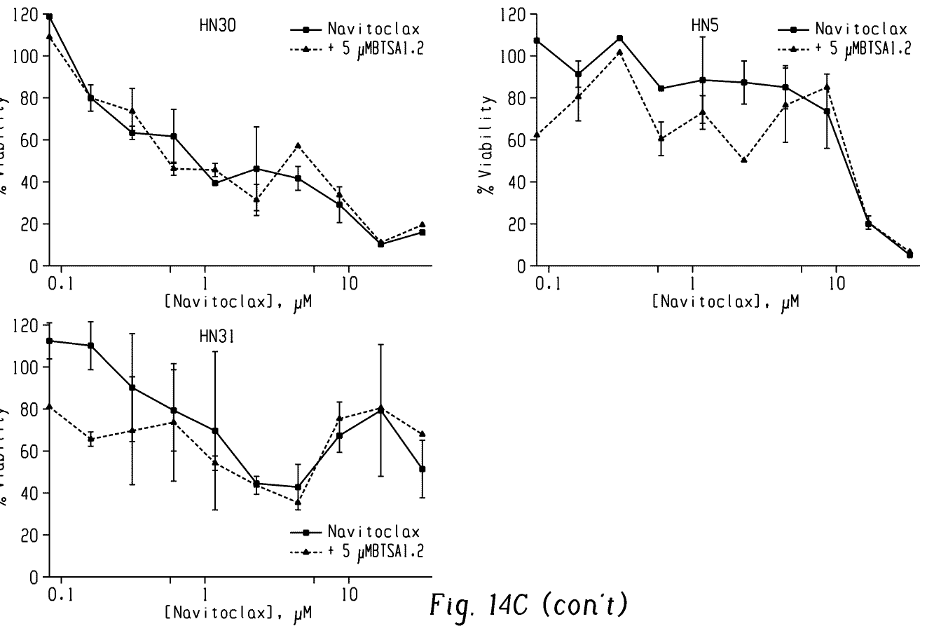

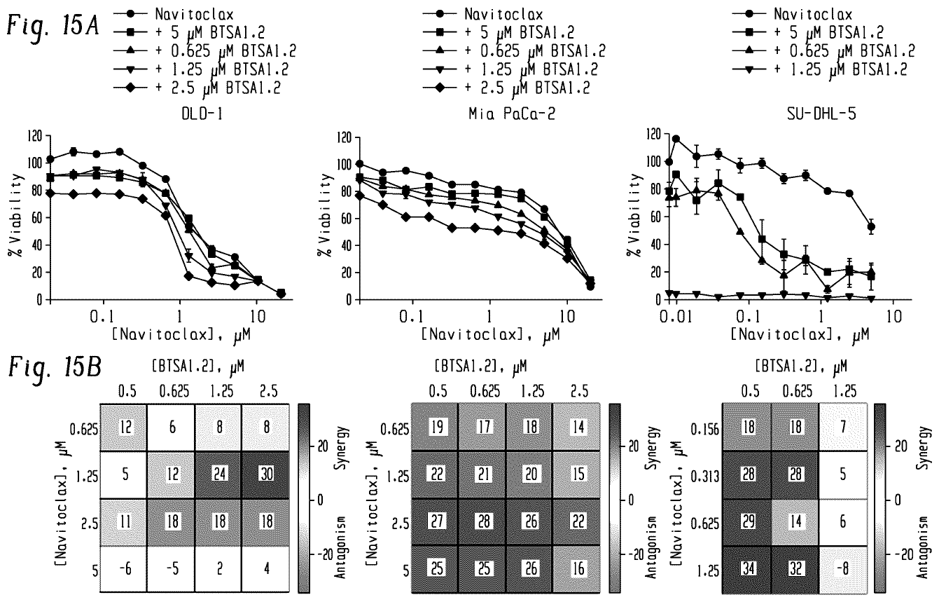

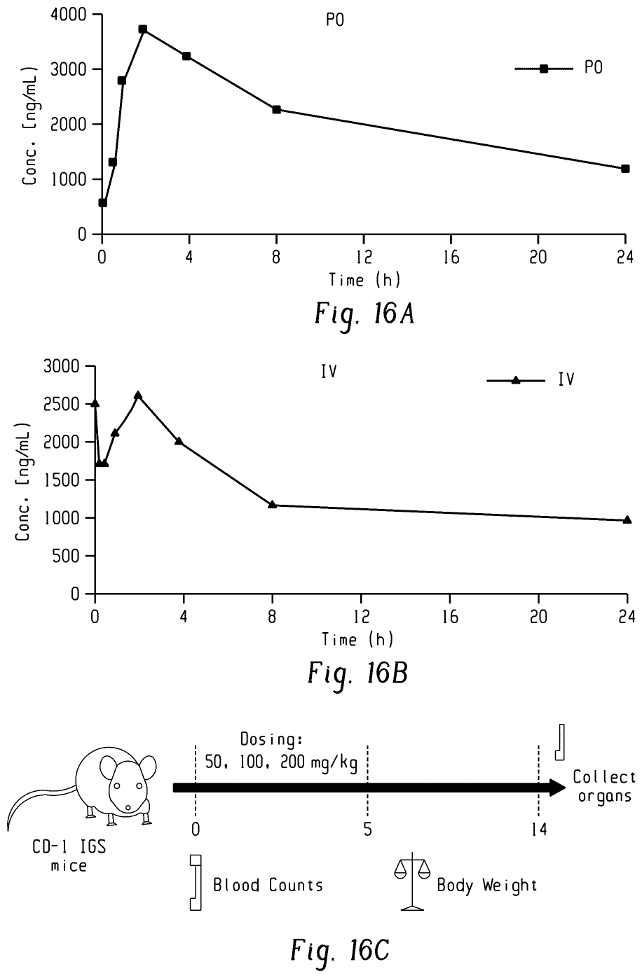

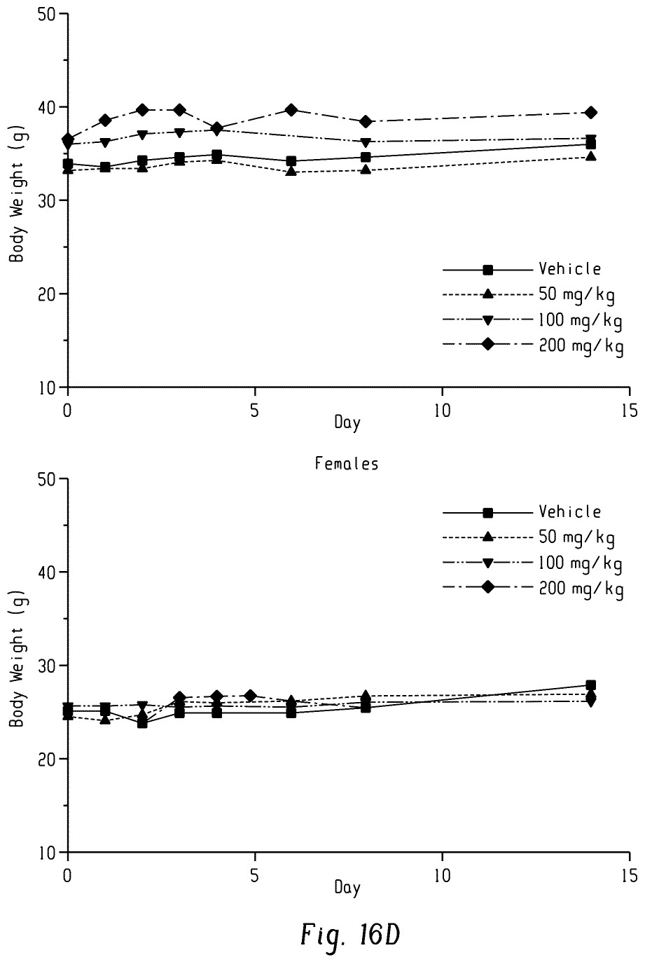

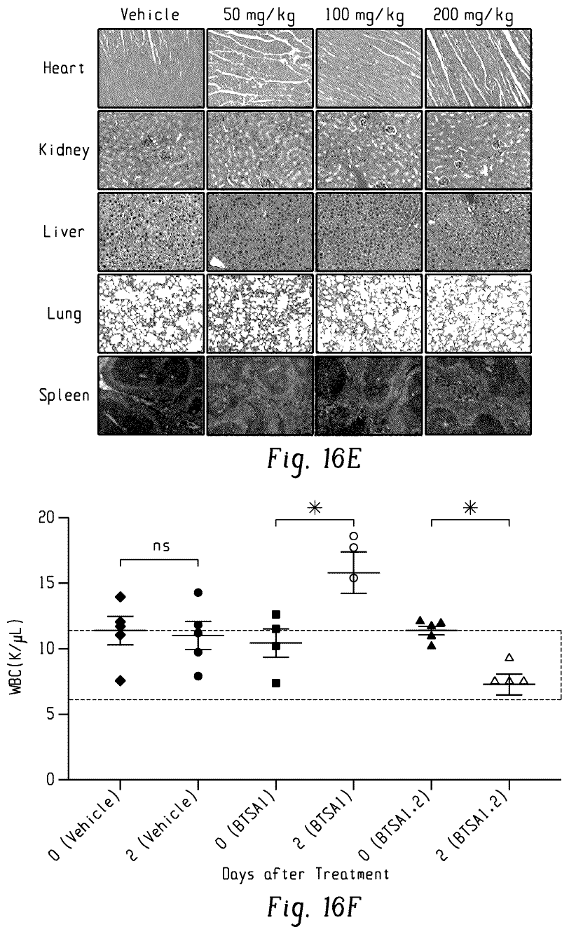

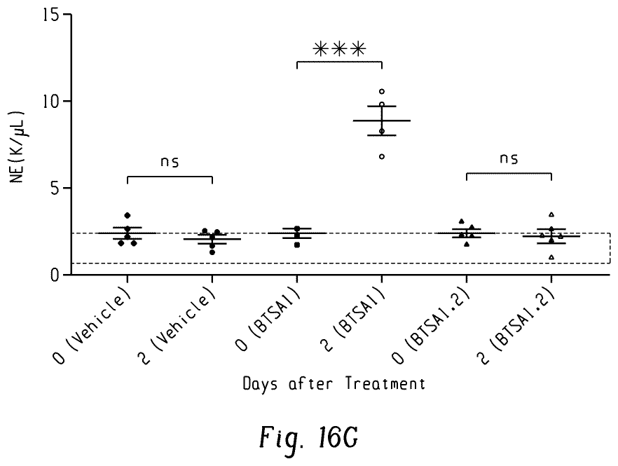

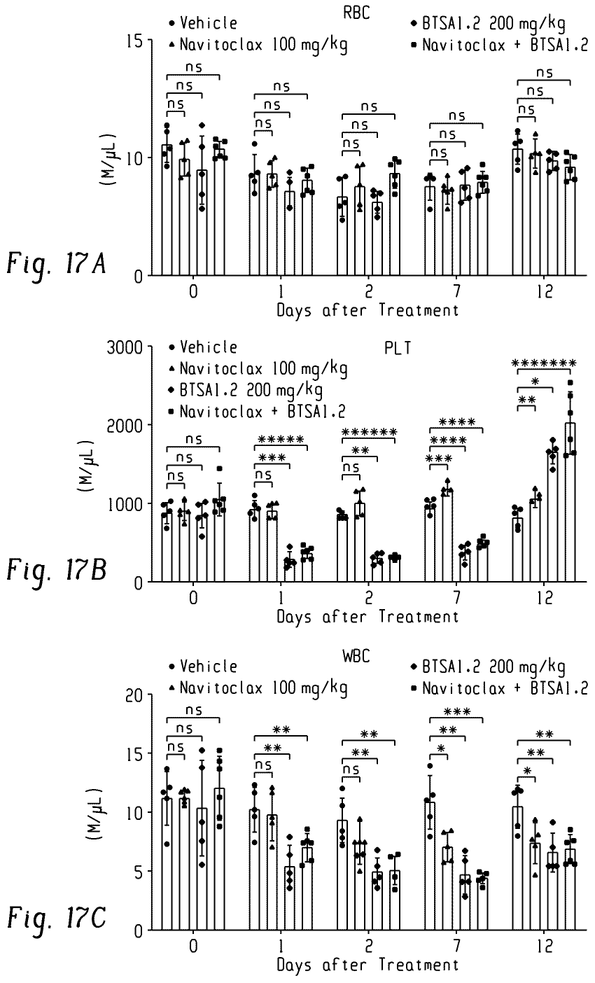

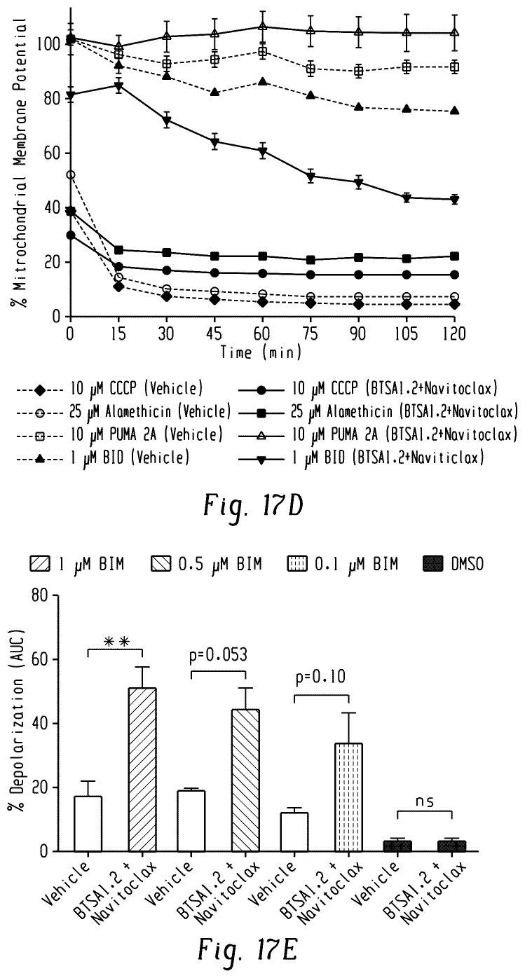

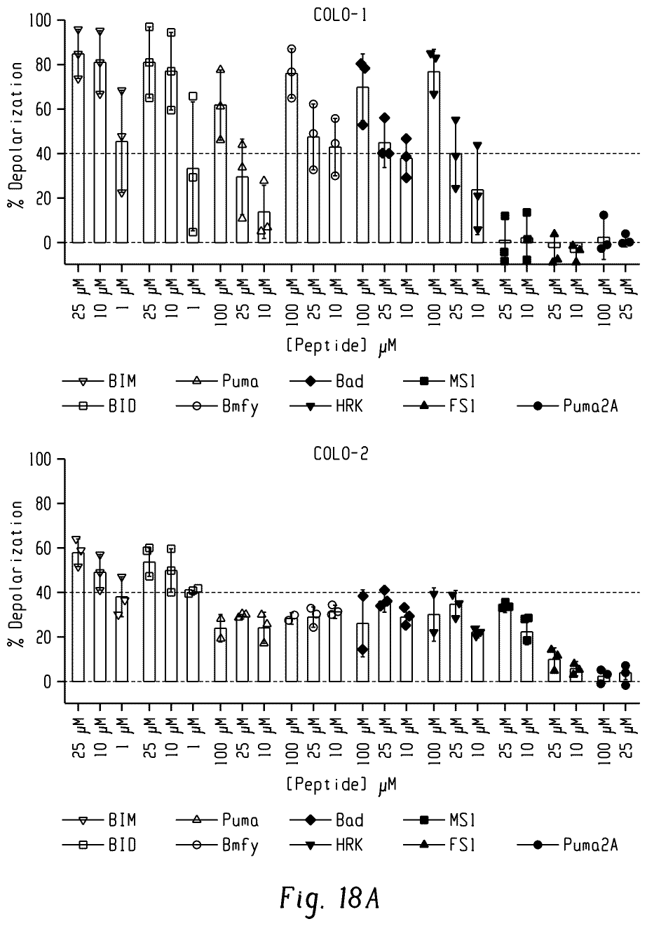

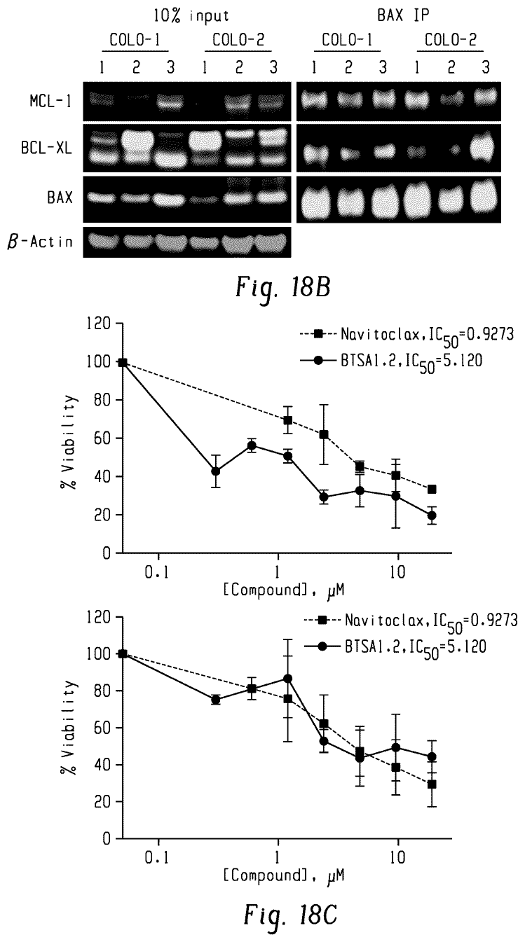

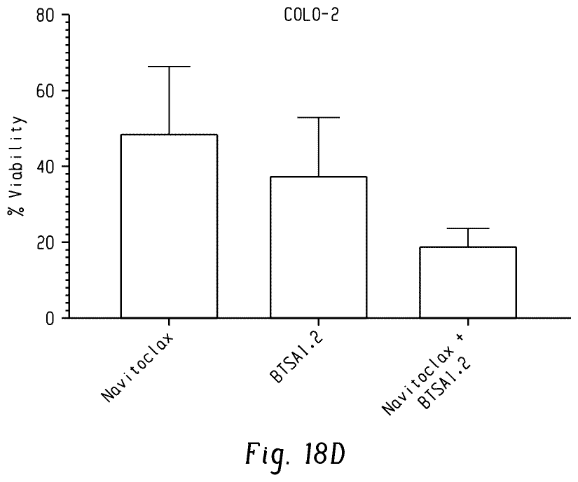

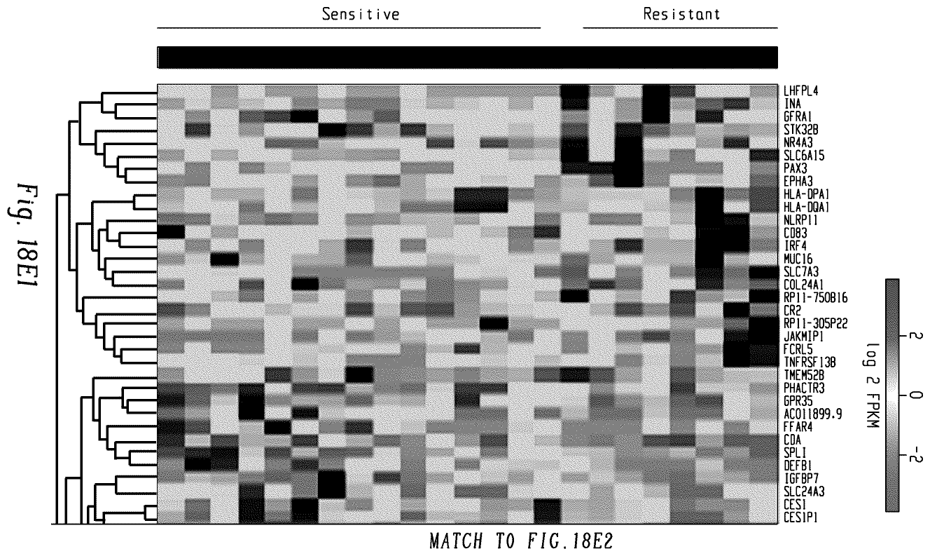

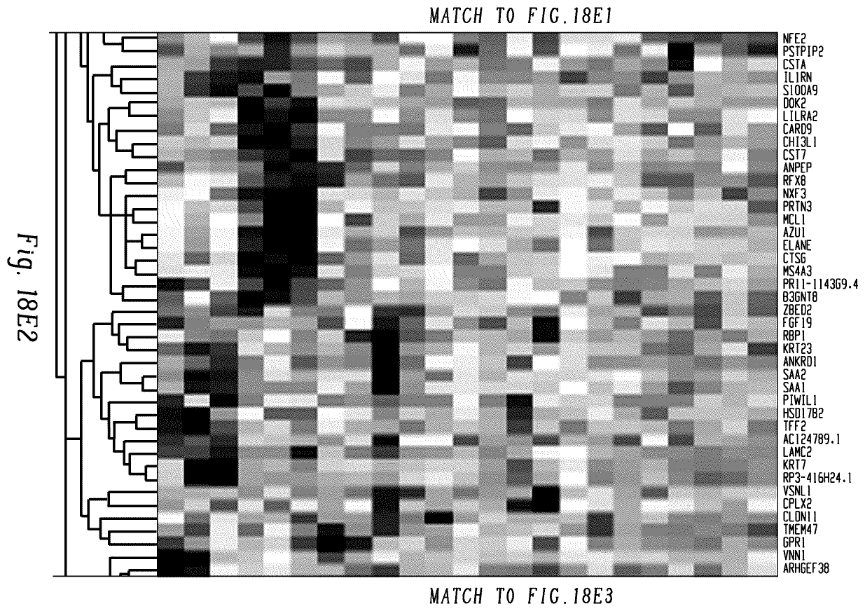

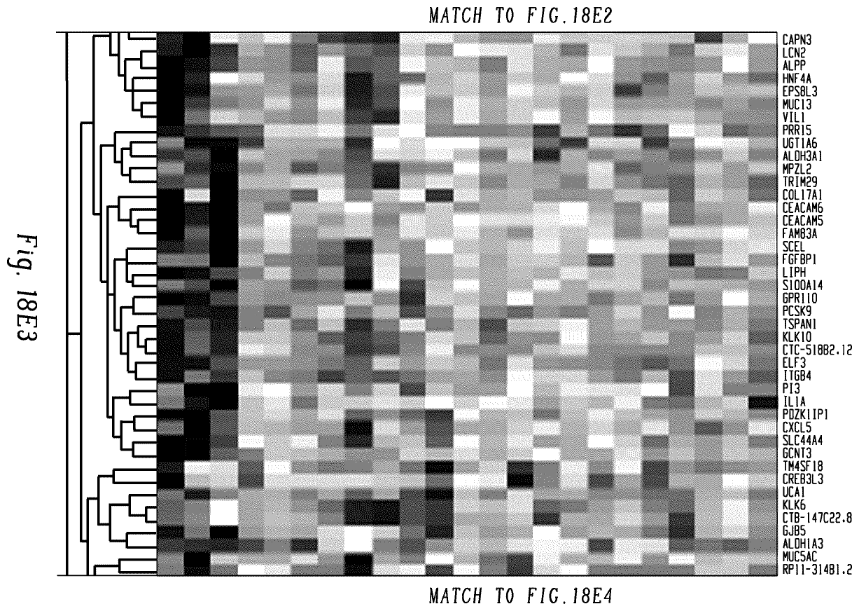

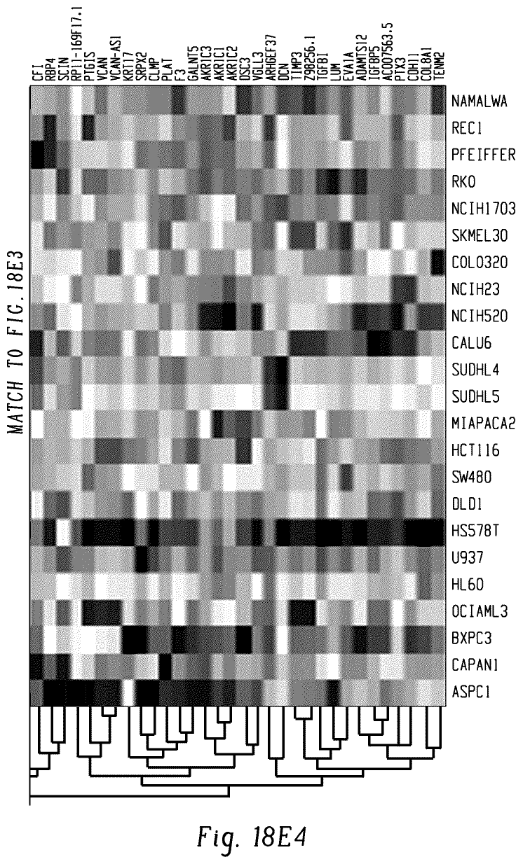

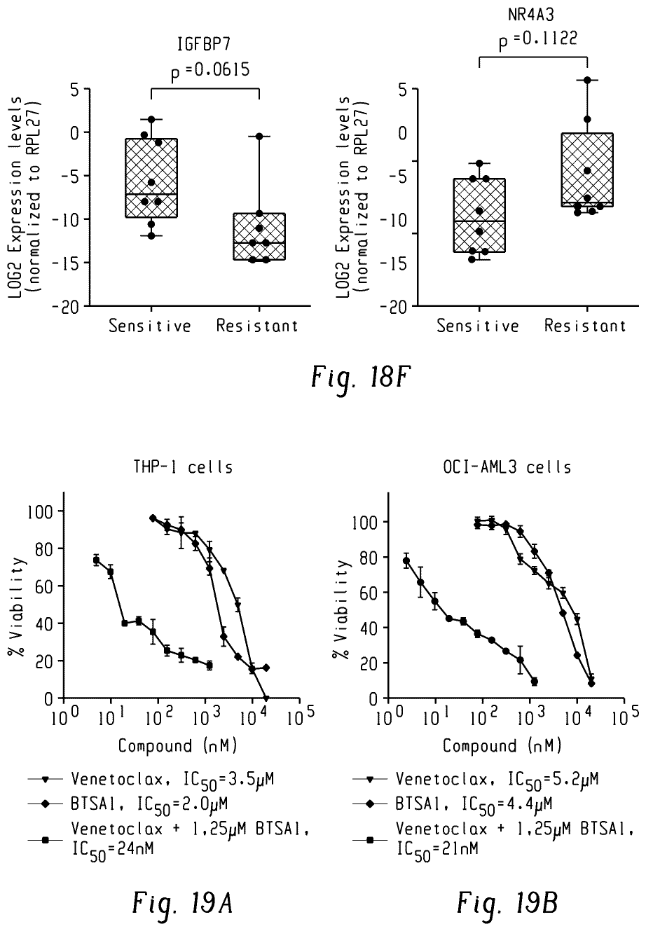

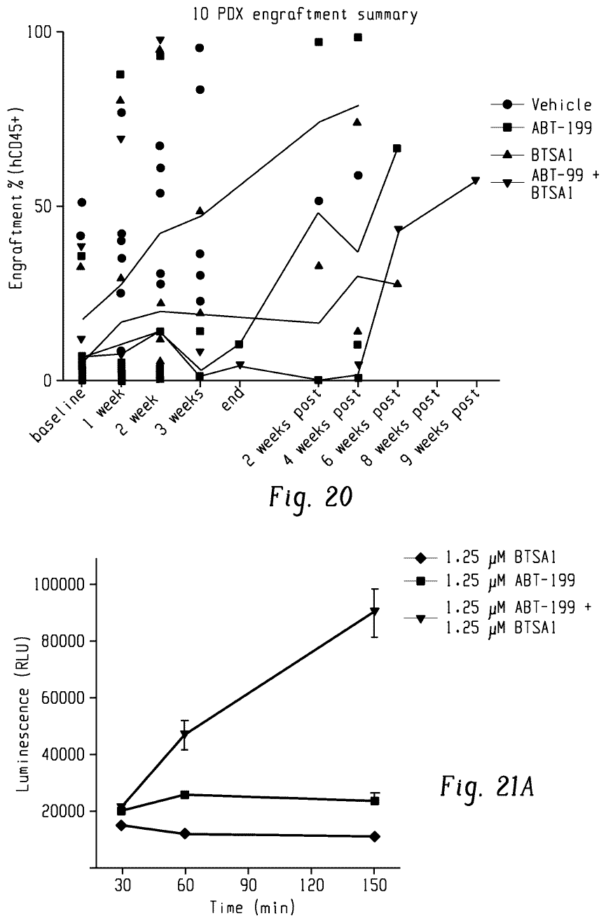

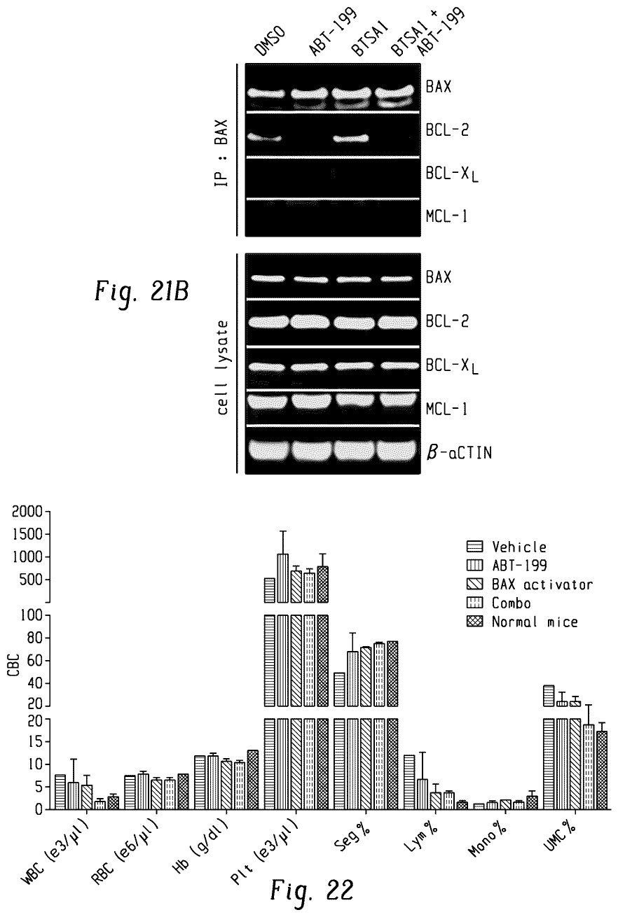

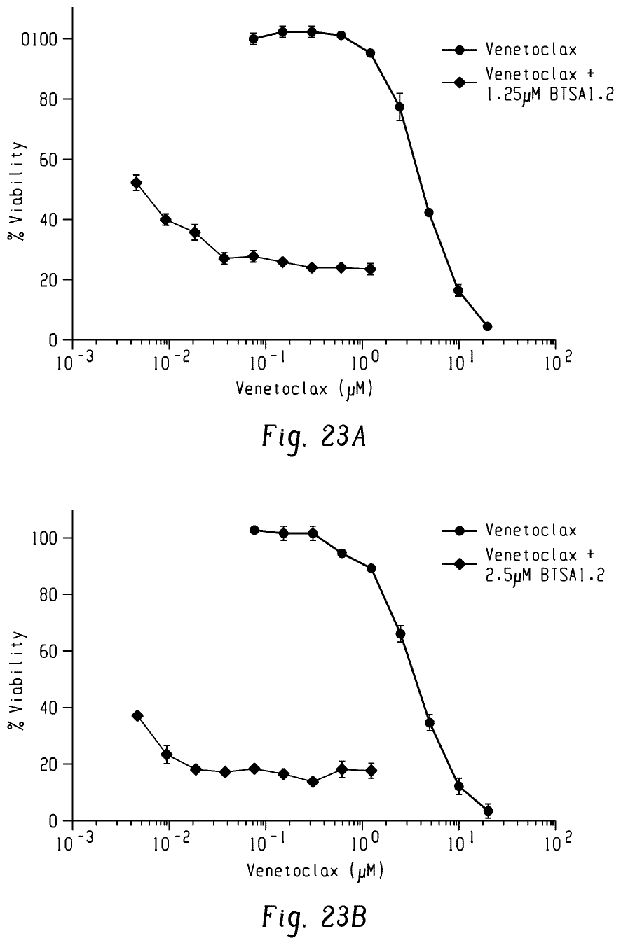

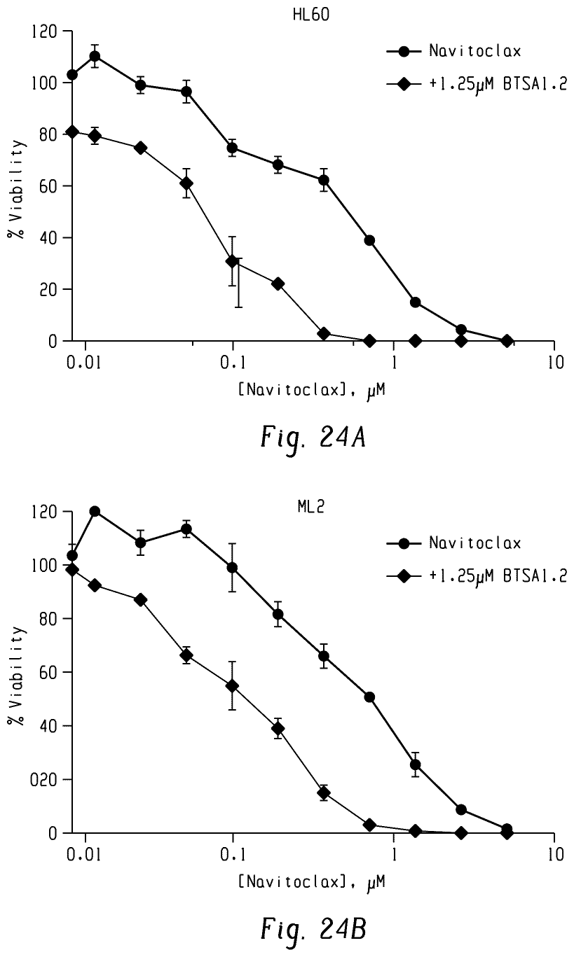

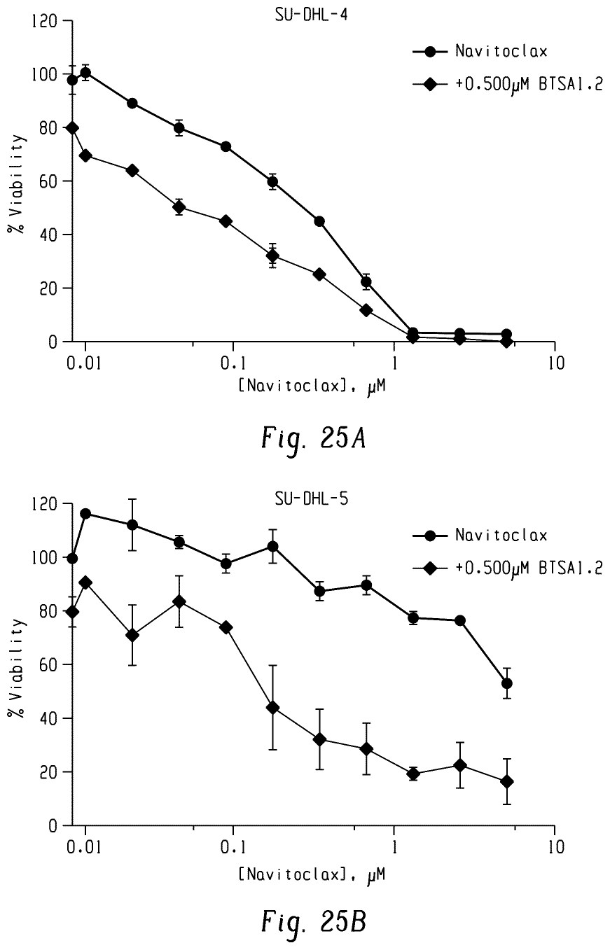

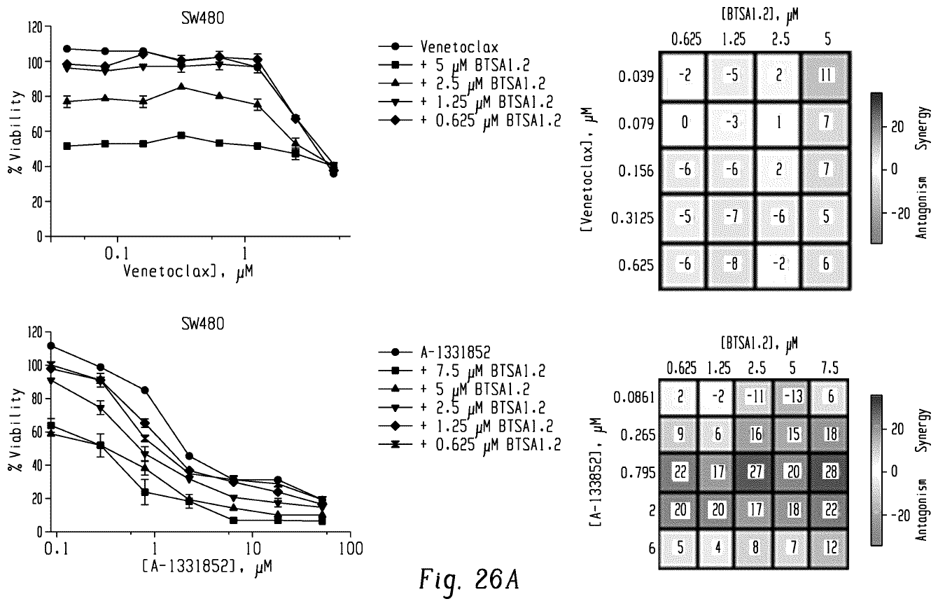

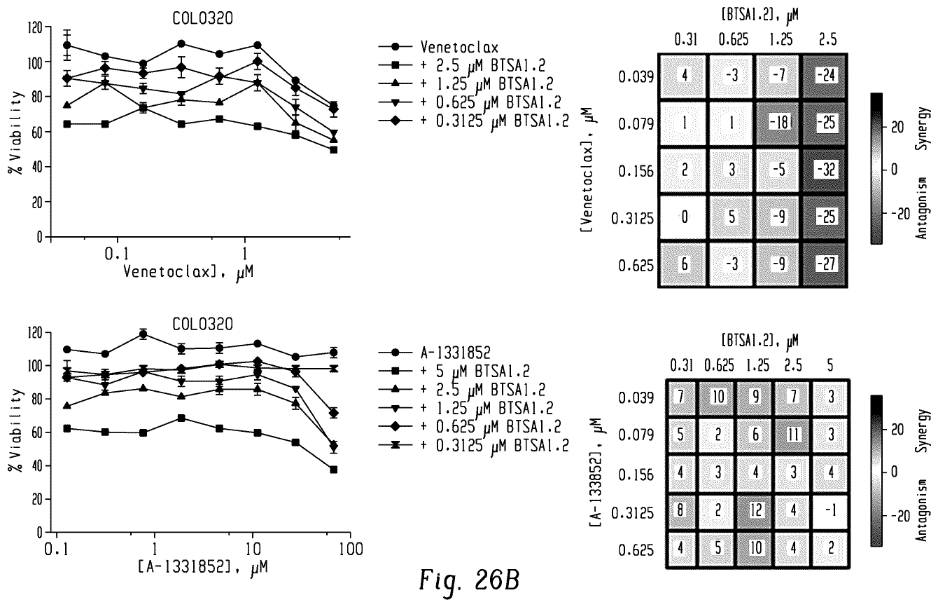

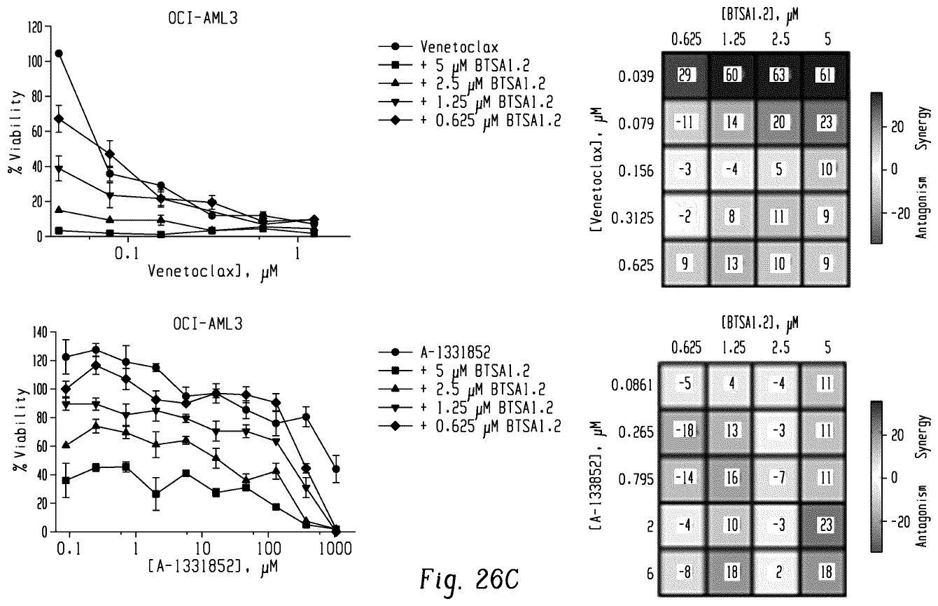

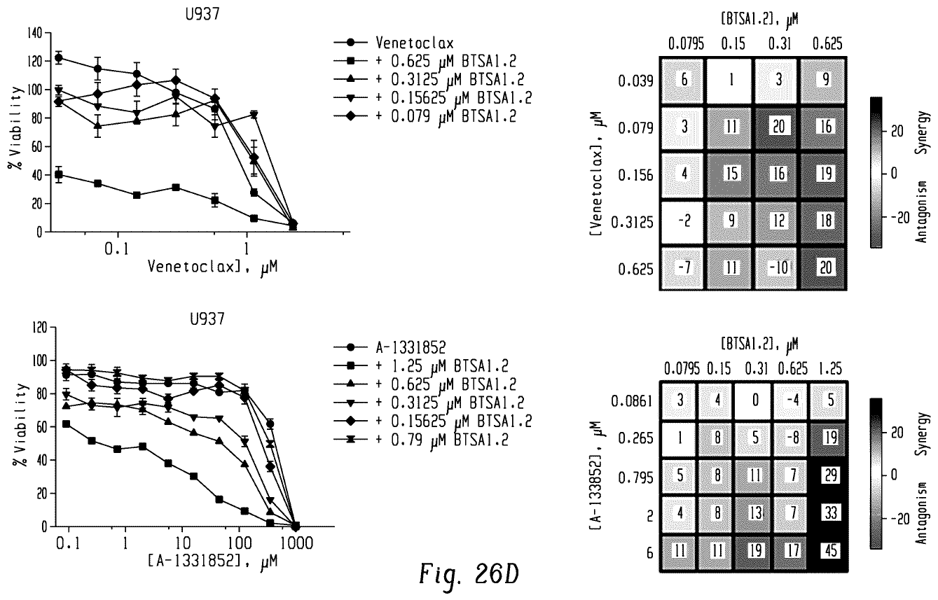

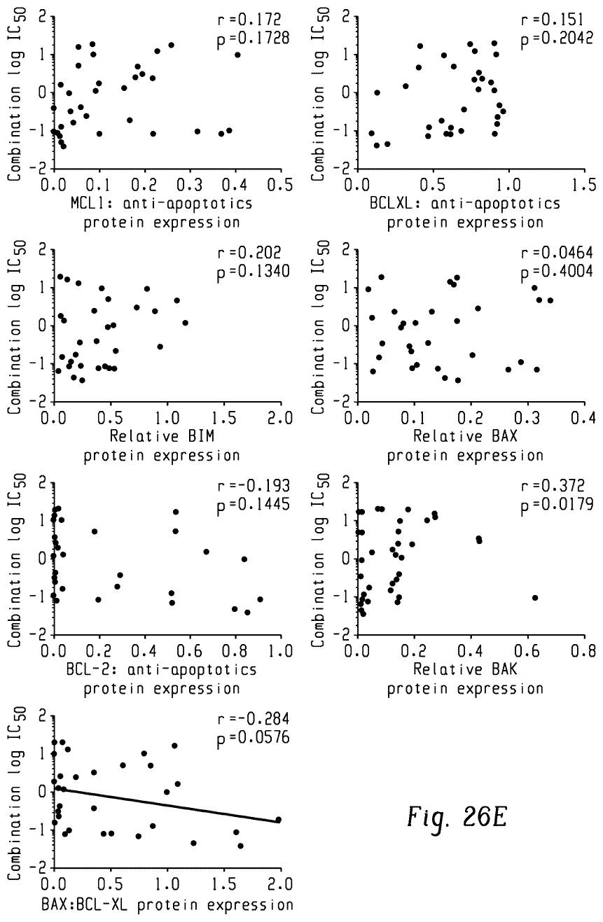

We then conducted a toxicity study for the BTSA1.2 and Navitoclax combination using their respective MTDs, 200 mg/kg/po for BTSA1.2 and 100 mg/kg/po for Navitoclax as previously determined by Tse et al, Cancer Res. 2008 May 1;68(9):3421-8 (FIG. 4A). While body weight, red blood cells counts and organs examined were between the normal parameters, lymphocyte, white blood cell and platelet counts reached levels below normal counts upon a single treatment with Navitoclax (FIGS. 4B-4G), as previously described by Tse et al, Cancer Res. 2008 May 1;68(9):3421-8 and Whitecross et al, Blood. 2009 Feb 26;113(9):1982-91. Upon BTSA1.2 treatment body weight and blood counts were measured in normal levels but reduction in white blood cells and lymphocytes counts was observed after repeated dosing. BTSA1.2 co-administration with Navitoclax was well-tolerated and no additional toxicity was observed in body weight, organs, and bood counts compared to single agent treatment (Fog. 4B-4G). Furthermore, Navitoclax/BTSA1.2 treated mice looked healthy upon treatment and daily monitoring, and normal blood counts were reached after concluding the treatment (FIGS. 17A-17C). The lack of toxicity in BTSA1.2 treated mice alone and in combination with Navitoclax is favorable compared to previous BH3 mimetics and their combinations e.g. combination of BCL-2 inhibitor and MCL-1 inhibitor, which demonstrated additional toxicities in blood counts compared to the single agents. Collectively, the data suggests that the BTSA1.2 and Navitoclax combination is well tolerated and safe to use in vivo.

### BTSA1.2 AND NAVITOCLAX COMBINATION IS EFFICACIOUS IN RESISTANT COLORECTAL XENOGRAFTS

BCL-XL plays a key role in colorectal tumors formation and therapy resistance. However, there is no clinical testing of BH3-mimetics for colorectal tumors as preclinical studies suggest that only BCL-XL inhibition is not sufficient to effectively induce apoptosis. Here, we found that BNc was able to promote apoptosis in colorectal tumors in vitro. To evaluate the therapeutic efficacy of the BTSA1.2 and Navitoclax combination in vivo, we selected to evaluate colorectal SW480 cells in xenograft mouse models, since SW480 cells were resistant to either BTSA1.2 or Navitoclax treatments.

Once xenografts were established, mice were randomly divided into four groups for treatment with vehicle, BTSA1.2, Navitoclax and the combination. Treatments started when tumors reached a volume of ˜200 mm³ using a daily oral administration with a MTD dose (FIG. 5A). While BTSA1.2 or Navitoclax as single agents had no significant efficacy in reducing tumor growth, oral co-administration of BTSA1.2 and Navitoclax was able to significantly suppress tumor growth compared to vehicle or single agent treatment, accounting for the synergistic activity of the two drugs in vivo (FIGS. 5B-D). Importantly, body weights remained constant during the in vivo study period and mice appeared healthy after treatment with the compounds (FIG. 5B).

To further confirm the synergistic efficacy of the two pro-apoptotic drugs in vivo, we treated each group of mouse xenografts for only three days after tumors reaching a volume of ˜400 mm³ and tumors were isolated for assessing several apoptotic markers such as caspase-3 cleavage, PARP cleavage and mitochondrial depolarization (FIG. 5E). Consistent with the tumor growth data, we determined that only tumors treated with the BTSA1.2 and Navitoclax combination exhibited significantly elevated apoptotic markers compared to vehicle or single agent treatments (FIG. 5F-I). Taken together, our data indicated that the BTSA1.2 and Navitoclax combination is synergistically efficacious and well tolerated in vivo.

### FUNCTIONAL MARKERS IDENTIFY SENSITIVE PATIENT COLORECTAL TUMORS TO THE COMBINATION

The data indicated that cell lines categorized as sensitive to the BTSA1.2 and Navitoclax combination (BNc) were characterized as anti-apoptotic BCL-XL dependent or “unprimed” by BH3-profiling, and formed increased levels of BAX:BCL-XL complexes than cell lines resistant to the combination (FIGS. 3B-3E). Since these functional assays distinguished cancer cell lines sensitive and resistant to the BNc, we evaluated if these functional assays could be also used to predict efficacy of the BNc in patient-derived xenografts (PDX) samples (FIG. 6A). Two colorectal patient-derived xenografts (PDX) samples (FIG. 6A), COLO-1 and COLO-2, were analyzed. PDX samples were analyzed by quantitative co-immunoprecipitation of BAX with BCL-XL and results indicated that both PDX had similar levels of BAX:BCL-XL complexes. However, BH3 profiling of the PDX samples designated COLO-1 as BCL-XL dependent while COLO-2 was characterized as “unprimed” for apoptosis (FIGS. 6B-C, FIGS. 18A-18B). As BNc was effective in BCL-XL dependent and unprimed to apoptosis cancer cells, it was predicted that COLO-1 and COLO-2 PDX should be sensitive to the BTSA1.2 and Navitoclax combination. Indeed, consistently with enhanced pro-apoptotic activity, treatment of PDXs ex vivo showed that the BTSA1.2 and Navitoclax combination (BNc) induced increased loss of viability when compared to single agents in both PDX samples (FIG. 6D, FIGS. 18C-14D).

Next, the therapeutic efficacy of the BTSA1.2 and Navitoclax combination was evaluated using an in vivo using mouse PDX model from COLO-1 tumor. After PDX models were established, mice were randomly divided into four groups for treatment with vehicle, BTSA1.2, Navitoclax and the combination, and treatments started when tumors reached a volume of ˜200 mm³ (FIG. 6E). Compounds were administered orally, once daily, using for BTSA1.2 the MTD, while this time a less toxic dose (half the MTD) of Navitoclax was tested as single agent and combination treatment. Treatments continued for up to 18 days or until tumor size reach an ethically unacceptable levels and then mice were monitored to evaluate survival (FIG. 6E). Combination of BTSA1.2 with a less toxic dose of Navitoclax was able to significantly suppress tumor growth and achieve tumor regression more than vehicle, BTSA1.2 or Navitoclax treatment, accounting for the synergistic activity of the two drugs in vivo (FIGS. 6F-6G). Notably, some PDX showed to respond to BTSA1.2 only treatment as their tumor growth was suppressed.

Consistent with the tumor growth data, combination of BTSA1.2 and Navitoclax was also able to significantly increase survival compared to vehicle or single agent treatments after termination of treatment (FIG. 6H). Moreover, PDX tumors treated with the drug combination had increased mitochondrial depolarization, increased apoptotic priming, compared to vehicle treatment PDX, which confirmed the pro-apoptotic efficacy of the combination (FIG. 6I). Of note, body weights remained constant during the in vivo study period and mice appeared healthy after treatment with the compounds (FIG. 6F). Interestingly, tumors of mice treated with single agent BTSA1.2 or Navitoclax had a significant increase of BCL-XL protein levels while MCL-1 levels remained constant (FIG. 6J). This analysis further supports the in vitro data which indicated that BCL-XL upregulation confers resistance to single agent BTSA1.2 or Navitoclax treatment (FIG. 1, 3C, 3D).

In addition, we evaluated COLO-2 PDX in vivo to determine whether PDX sample characterized as unprimed for apoptosis with BAX:BCL-XL complexes are indeed sensitive to the BTSA1.2 and Navitoclax combination in vivo (FIGS. 6B-6C, FIGS. 18C-18D). Similarly to the COLO-1 PDX studies, the combination of BTSA1.2 with a less toxic dose of Navitoclax was able to significantly suppress COLO-2 tumor growth and achieve tumor regression more than vehicle, BTSA1.2 or Navitoclax treatment, accounting for the synergistic activity of the two drugs in vivo (FIGS. 6J-6K).

Taken together, we were able to predict sensitivity to the BTSA1.2 and Navitoclax combination based on BH3-profiling and BAX co-immunoprecipitation with BCL-XL. Our data suggest that BAX:BCL-XL complexes as well as “BCL-XL dependent” and “unprimed for apoptosis” could be useful as sensitivity markers for this combination therapy. Importantly, these studies demonstrated the therapeutic efficacy of the combination in colorectal PDX models using even a lower dose for Navitoclax that has less toxicity on platelet counts.

### GENOMIC MARKERS PREDICT SENSITIVITY OR RESISTANCE TO THE BTSA1.2 AND NAVITOCLAX COMBINATION

Identifying genomic biomarkers for sensitivity or resistance to the drug combination may provide information that could be useful for patient selection and further biological investigation. Having evaluated the BTSA1.2 and Navitoclax combination in a diverse panel of solid tumors and hematologic malignancies (FIG. 2A, 2B) and realizing the significant therapeutic efficacy of the combination in vivo in specific colorectal tumors (FIGS. 5C, 6J, 6K), we were interested to identify genomic markers that can predict tumors sensitive and resistant to the drug combination. We used genomic information and particularly gene expression analysis that is publicly available for several sensitive and resistant cell lines to the drug combination (FIG. 2B). Bioinformatics analysis identified significant differences in gene expression between sensitive and resistant groups and ˜250 hits were identified with high fold change and statistical significance (FIG. 7A, FIG. 18E). We examined the top differentially expressed genes for their potential association with apoptosis, resistance to current treatments and/or poor cancer prognosis using literature and patient database searches and selected several genes for further validation. Genes that were highly expressed in sensitive cell lines MUC13, EPS8L3 and IGFBP7 were predicted as potential markers of sensitivity to the BTSA1.2 and Navitoclax combination. On the other hand, genes such as NR4A3, IRF4 and SLC7A3 were highly expressed in resistant cell lines, suggesting these genes could be used as markers of resistance to the combination (FIG. 7A, FIG. 18E).

To confirm the correlation for these specific genes we selected sensitive and resistant cell lines to the BTSA1.2/Navitoclax combination, and confirmed the higher expression either in sensitive or resistance cell lines by RT-qPCR (FIG. 7B). Further analysis showed that the expression levels of the cell surface receptor gene MUC13 showed significant correlation with BCL-XL gene expression levels (FIG. 7C). Therefore, higher expression of the MUC13 marker correlates with the upregulation of BCL-XL, which also determines sensitivity to the BTSA1.2/Navitoclax combination. Notably, analysis of patient tumor data indicates that colorectal cancers and other solid tumors, such as pancreatic and stomach cancers, have higher levels of MUC13, suggesting that patients with tumors having high MUC13 levels could be the ones to benefit most from treatment with the BTSA1.2 and Navitoclax combination (FIG. 7E). Likewise, uveal melanoma, thyroid and thymoma tumors could be most resistant to the combination treatment (FIG. 7D). Taken together, we were able to identify genomic markers to the BTSA1.2 and Navitoclax combination based on genome-wide bioinformatics, which could be used as sensitivity or resistance markers for this combination therapy.

### DISCUSSION

Numerous studies have established the critical role of BCL-2 family proteins in regulating apoptosis in tumor development, maintenance, and resistance to targeted therapies and chemotherapy. Frequently, upregulation of the major anti-apoptotic members BCL-2, BCL-w, BFL-1, BCL-XL and MC1-1 block pro-apoptotic members and apoptosis. Potent and selective inhibitors of these proteins, termed BH3 mimetics, have been developed. These drugs have demonstrated activity against various hematologic malignancies. Indeed, Venetoclax, a selective BCL-2 inhibitor, has been the first drug approved for a subset of patients with Chronic Lymphocytic Leukemia or Acute Myeloid Leukemia. Despite this success, several studies have shown solid tumors are largely refractory to these drugs when used as single agents. For these more resistant tumors either multiple anti-apoptotic BCL-2 proteins are upregulated and/or pro-apoptotic BH3-only proteins that are required to activate BAX and BAK for induction of apoptosis are kept suppressed.

The development of small molecules that directly activate BAX to induce apoptosis represent a major advance in our ability to promote apoptosis in cancer cells. BAX activators can drive cancer apoptosis or potentiate apoptotic priming and are not dependent on the availability of BH3-only protein activators. Here, we described BTSA1.2, an improved small molecule BAX activator from previously described BTSA1, which has increased potency, oral bioavailability and is well tolerated in vivo. BTSA1.2 against a diverse range of solid tumors and hematologic malignancies demonstrated significant activity in leukemia and lymphoma cell lines, but similar to other BH3 mimetics, the efficacy of BTSA1.2 in the tested solid tumor cell lines was reduced.

Our studies suggest that higher protein levels of BAX correlate with increased pro-apoptotic activity of BTSA1.2. The BTSA1.2 activity in leukemia cells is consistent with the significant efficacy as single agent therapy as demonstrated for BTSA1 in human AML models. This is consistent with the mechanism of BAX activation as increased BAX protein levels can lead to more activated BAX by the small molecule BAX activator, and therefore to increased mitochondrial outer membrane (MOMP) permeabilization and apoptosis induction. Moreover, data also suggest that BCL-XL is a primary regulator of the BAX activation response. BCL-XL and BCL-2 have higher affinity for BAX compared to MCL-1. Therefore, in the absence of BCL-2 expression as evidenced in the majority of solid tumor cell lines, BCL-XL is the primary anti-apoptotic protein to sequester activated BAX. When both BCL-XL and BCL-2 proteins are expressed at similar levels, as evidenced mainly in hematological malignancies, then both proteins can regulate BAX activation. Our testing of Navitoclax against the same diverse cell lines suggested that Navitoclax activity is primarily dependent on the levels of BAX and that targeting only BCL-XL in several cell lines is not enough to promote apoptosis. Furthermore, BH3-profiling highlighted that the majority of cells resistant to either BTSA1.2 or Navitoclax show dependency to BCL-XL or they are unprimed for apoptosis. Thus, our investigation of the mechanisms of apoptotic resistance in a range of malignancies, uncovered two survival mechanisms that limit direct and indirect BAX activation and apoptosis induction.

The combination of the BTSA1.2, and Navitoclax demonstrated synergistic activity in diverse solid tumor and hematologic cell lines that commonly have dependency on BCL-XL inhibition or they are unprimed to apoptosis. Of note, this synergistic activity was not affected by common oncogenic mutations such as TP53 or KRAS which typically limit the efficacy of chemotherapeutics and targeted therapies in cancer. Therefore, the combination of BTSA1.2 and Navitoclax could be applied more broadly to a variety of tumors.

Dissecting this mechanism of combined BAX activation and BCL-XL inhibition in the sensitive cell lines, we found that BAX:BCL-XL complexes are formed without treatment in the cell lines sensitive to the combination. Activation of cytosolic BAX by BTSA1.2 can promote additional BAX:BCL-XL complexes making these cells more primed to anti-apoptotic inhibition by Navitoclax or a BCL-XL selective inhibitor. On the other hand, Navitoclax or a BCL-XL selective inhibitor is capable to break BAX:BCL-XL complexes directly or indirectly using derepressed BH3-only proteins. In these cases, apoptotic activity from BCL-XL inhibition will depend on the levels of activated BAX bound to BCL-XL and the levels of BH3-only proteins bound to BCL-XL that can be derepressed to activate BAX. Therefore, the combined activity of a BAX activator and a BCL-XL inhibitor offers an effective strategy to induce apoptosis, by concurrently increasing the levels of activated BAX and inhibiting sequestration of activated BAX by BCL-XL, to enable increased MOMP and apoptosis induction.

The combination of the BTSA1.2 and Navitoclax also demonstrated synergistic therapeutic efficacy in colorectal tumors while also it was remarkably tolerated in vivo. Indeed, this therapeutic strategy may be highly promising for colorectal tumors considering previous compelling evidence that high expression levels of BCL-XL play key role in colorectal tumors formation and therapy resistance. Despite these evidences, application of Navitoclax in colorectal tumors, including our work here, suggests that BCL-XL inhibition is not sufficient as a single agent treatment and is not able to effectively drive apoptosis. Previous studies have shown Navitoclax to synergize effectively with targeted therapies such as EGFR inhibitors in non-small lung cancer and MEK inhibitors in KRAS mutant cancers and BRAF mutant melanoma. These studies showed that targeting oncogenic driver pathways lead to increased BH3-only proteins, e.g. upregulation of BIM by MEK inhibitors, which enhance priming and efficacy of Navitoclax-mediated BCL-XL inhibition. Currently active clinicals trials are testing the efficacy of these combinations in patients (NCT03222609, NCT02079740, NCT01989585). However, these combination strategies rely on mutation of specific kinases to be effective. As in our studies we found that the mutational background of cancer cells did not affected the synergy between Navitoclax and BTSA1.2, this supports that this combination strategy could be effective in a variety of tumors. Furthermore, Navitoclax is hindered by its thrombocytopenia effect and a combination therapy in clinical trials will require an effective therapeutic window. Therefore, the fact that our combination studies in vivo demonstrate synergistic therapeutic efficacy with a reduced Navitoclax dose and overall a safe profile in tissues and blood counts, is noteworthy. In addition, efforts to develop clinical compounds that target BCL-XL with minimal toxicity on platelets are underway and these could be used alternatively to potentiate the pro-apoptotic activity of BTSA1.2.

Our work identified functional assays and markers to predict sensitivity on concurrent BAX activation and BCL-XL inhibition, based on BAX:BCL-XL complexes and BH3-profiling of cancer cells. Development of diagnostic assays for identification of BAX-containing protein complexes and BH3 profiling from solid tumor biopsies should be established next. Although this remains to be determined, our data on genomic analysis and identification of genes for sensitivity or resistance to the drug combination provide information that may be useful for biomarker selection. Our analysis identified high levels of MUC13 to be a marker of sensitivity to the BTSA1.2 and Navitoclax combination, suggesting that this combination therapy could be beneficial for cancer patients with high levels of MUC13. Interestingly, MUC13 has been proposed as a marker of poor prognosis in colorectal tumors supporting our findings for combined targeting of BAX and BCL-XL in resistant colorectal tumors. Furthermore, from a mechanistic stand-point our bioinformatic analysis provides stimulating data for future studies to analyze the impact and relationship of markers such as MUC13 to regulate the expression and interactions among the BCL-2 protein family and apoptosis induction.

In summary, the data herein advances the understanding of cell death mechanisms in cancer cells and demonstrates a novel therapeutic strategy, which rationally targets pro-apoptotic BAX and anti-apoptotic BCL-XL to overcome apoptosis resistance mechanisms in a range of tumors. Our findings provide preclinical proof-of-concept for the combination of a new BAX activator, BTSA1.2, and Navitoclax, which may provide a broad therapeutic effect in tumors.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A pharmaceutical combination, comprising:
a B-cell lymphoma 2 associated X protein (BAX) activating compound; and
an anti-apoptotic protein inhibiting compound.

2. The pharmaceutical combination of claim 1, wherein the anti-apoptotic protein inhibiting compound is a BCL-XL inhibiting compound, a BCL-2 inhibiting compound, a BCL-w inhibiting compound, a BFL-1 inhibiting compound or a MCL-1 inhibiting compound.

3. The pharmaceutical combination of claim 1, wherein the BAX activating compound is a compound having a structure of BTSA1.2, or a pharmaceutically acceptable salt thereof

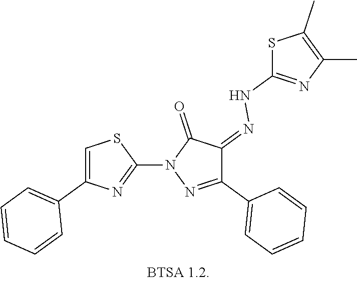 [SMILES: Cc1nc(N/N=C2\C(=O)N(c3nc(-c4ccccc4)cs3)N=C2c2ccccc2)sc1C]

4. (canceled)

5. The pharmaceutical combination of claim 1, wherein the anti-apoptotic protein inhibiting compound is ABT-737, navitoclax, venetoclax, AMG 176, AMG 397, AZD-4320, AZD-0466, AZD-5991, VU661013, 565487, MIK665, sabutoclax, gambogic acid, obatoclax mesylate, APG1252, DT2216 or a combination of any of the foregoing.

6. A pharmaceutical composition comprising the pharmaceutical combination of claim 1, and a pharmaceutically acceptable carrier.

7. A method of treating a cancer in a subject comprising administering to the subject a B-cell lymphoma 2 associated X protein (BAX) activating compound in combination with a B-cell lymphoma-extra large protein (BCL-XL) inhibiting compound, a BCL-2 inhibiting compound, a BCL-w inhibiting compound, a BFL-1 inhibiting compound or a MCL-1 inhibiting compound in an amount effective to treat the cancer in the subject.

8. The method of claim 7, wherein the BAX activating compound is a compound having a structure of BTSA1.2, or a pharmaceutically acceptable salt thereof.

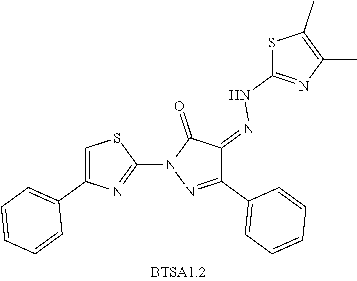 [SMILES: Cc1nc(N/N=C2\C(=O)N(c3nc(-c4ccccc4)cs3)N=C2c2ccccc2)sc1C]

9. The method of claim 7, wherein the anti-apoptotic protein inhibiting compound comprises navitoclax or ventoclax.

10. The method of claim zany of claims 7, wherein the cancer is a hematological cancer or a solid tumor.

11. The method of claim 10, wherein the cancer is breast cancer, prostate cancer, lymphoma, skin cancer, pancreatic cancer, colon cancer, rectal cancer, colorectal cancer, melanoma, malignant melanoma, ovarian cancer, brain or spinal cord cancer, primary brain carcinoma, medulloblastoma, neuroblastoma, glioma, head-neck cancer, glioma, glioblastoma, liver cancer, bladder cancer, stomach cancer, kidney cancer, placental cancer, cancer of the gastrointestinal tract, non-small cell lung cancer (NSCLC), head or neck carcinoma, breast carcinoma, endocrine cancer, eye cancer, genitourinary cancer, cancer of the vulva, ovary, uterus or cervix, hematopoietic cancer, myeloma, leukemia, lymphoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, chronic granulocytic leukemia, acute granulocytic leukemia, acute myelogenous leukemia, chronic myelogenous leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft tissue cancer, soft-tissue sarcoma, osteogenic sarcoma, sarcoma, primary macroglobulinemia, central nervous system cancer and retinoblastoma.

12. (canceled)

13. The method of claim 7, wherein a route of the administering comprises oral, rectal, sublingual, buccal, intravenous, intramuscular, transdermal, cutaneous, subcutaneous, intrathecal, nasal, vaginal, or a combination thereof.

14. (canceled)

15. A method of treating cancer in a subject in need thereof, the method comprising:
obtaining a biological sample comprising cancer cells from the subject;
detecting a level of BAX:anti-apoptotic protein complexes immunoprecipitated from the cancer cells and/or detecting that the cancer cells are anti-apoptotic protein dependent or unprimed to apoptosis; and
administering to the subject the pharmaceutical combination of claim 1 in an amount effective to treat the cancer.

16. The method of claim 15, wherein the anti-apoptotic protein inhibiting compounds is a BCL-XL inhibiting compound, a BCL-2 inhibiting compound, a BCL-w inhibiting compound, a BFL-1 inhibiting compound or a MCL-1 inhibiting compound.

17. The method of claim 15, wherein the BAX:anti-apoptotic protein complexes are formed by co-immunoprecipitating BAX and the anti-apoptotic protein from the cancer cells.

18. The method of claim 17, wherein the BAX: anti-apoptotic protein complex is BAX:BCL-XL and the anti-apoptotic protein is BCL-XL; the BAX: anti-apoptotic protein complex is BAX:BCL-2 and the anti-apoptotic protein is BCL-2; the BAX: anti-apoptotic protein complex is BAX:BCL-w and the anti-apoptotic protein is BCL-w; the BAX:
anti-apoptotic protein complex is BAX:BFL-1 and the anti-apoptotic protein is BFL-1 or the BAX: anti-apoptotic protein complex is BAX:MCL-1 and the anti-apoptotic protein is MCL-1.

19. (canceled)

20. The method of any of claim 16, wherein the detecting that the cancer cells are anti-apoptotic BCL-XL, BCL-2, BCL-w, BFL-1 or MCL-1 dependent or unprimed to apoptosis comprises BH3 profiling.

21. (canceled)

22. A method of treating cancer in a subject in need thereof, the method comprising:
obtaining a biological sample from the subject;
measuring expression level of at least one gene in the biological sample, wherein the gene comprises MUC13, EPS8L3, IGFBP7, or a combination thereof; and
administering to the subject the pharmaceutical combination of claim 1 in an amount effective to treat the cancer.

23. The method of claim 22, wherein the anti-apoptotic protein inhibiting compound is a BCL-XL inhibiting compound, a BCL-2 inhibiting compound, a BCL-w inhibiting compound, a BFL-1 inhibiting compound or a MCL-1 inhibiting compound.

24. The method of claim 23, the method further comprises determining that the expression level of the gene MUC13, EPS8L3, IGFBP7, or a combination thereof in the biological sample is increased as compared to a control sample prior to administering the sample and the anti-apoptotic protein inhibiting compound is a BCL-XL inhibiting compound.

25. (canceled)

26. (canceled)

27. (canceled)

28. (canceled)


SEQUENCE LISTING (26 sequences)

SEQ ID NO: 1 - protein, 20 aa, Artificial sequence
  hBIM peptide
MRPEIWIAQELRRIGDEFNA

SEQ ID NO: 2 - protein, 21 aa, Artificial sequence
  hBID-Y peptide
EDIIRNIARHLAQVGDSMDRY

SEQ ID NO: 3 - protein, 25 aa, Artificial sequence
  mBAD peptide
LWAAQRYGRELRRMSDEFEGSFKGL

SEQ ID NO: 4 - protein, 21 aa, Artificial sequence
  HRK-y peptide
SSAAQLTAARLKALGDELHQY

SEQ ID NO: 5 - protein, 20 aa, Artificial sequence
  mNoxaA peptide
AELPPEFAAQLRKIGDKVYC

SEQ ID NO: 6 - protein, 20 aa, Artificial sequence
  Puma peptide
EQWAREIGAQLRRMADDLNA

SEQ ID NO: 7 - protein, 21 aa, Artificial sequence
  Bmf-Y peptide
HQAEVQIARKLQLIADQFHRY

SEQ ID NO: 8 - protein, 20 aa, Artificial sequence
  Puma2A peptide
EQWAREIGAQARRMAADLNA

SEQ ID NO: 9 - protein, 23 aa, Artificial sequence
  MS1 peptide
RPEIWMTQGLRRLGDEINAYYAR

SEQ ID NO: 10 - protein, 23 aa, Artificial sequence
  FS1 peptide
QWVREIAAGLRLAADNVNAQLER

SEQ ID NO: 11 - DNA, 20 bp, Artificial sequence
  primer
tgcaggtattggtgagtcgg

SEQ ID NO: 12 - DNA, 20 bp, Artificial sequence
  primer
acaaaagtatcccagccgcc

SEQ ID NO: 13 - DNA, 21 bp, Artificial sequence
  primer
cctaccaacccacattctcag

SEQ ID NO: 14 - DNA, 21 bp, Artificial sequence
  primer
tccctaacctatgacttcccc

SEQ ID NO: 15 - DNA, 20 bp, Artificial sequence
  primer
tgccatgcatccaattccca

SEQ ID NO: 16 - DNA, 23 bp, Artificial sequence
  primer
tatagctcggcaccttcaccttt

SEQ ID NO: 17 - DNA, 21 bp, Artificial sequence
  primer
gtgaaaatggttgccaggtga

SEQ ID NO: 18 - DNA, 20 bp, Artificial sequence
  primer
aggcttcggcagaccttatg

SEQ ID NO: 19 - DNA, 21 bp, Artificial sequence
  primer
gtaaccagactgcggatgact

SEQ ID NO: 20 - DNA, 21 bp, Artificial sequence
  primer
agactggaagcaacgcagaaa

SEQ ID NO: 21 - DNA, 20 bp, Artificial sequence
  primer
gctgggcagaaaagattccg

SEQ ID NO: 22 - DNA, 21 bp, Artificial sequence
  primer
cagcagtgtttgacctgatgg

SEQ ID NO: 23 - DNA, 20 bp, Artificial sequence
  primer
catgggcaagaagaagatcg

SEQ ID NO: 24 - DNA, 20 bp, Artificial sequence
  primer
tccaaggggatatccacaga

SEQ ID NO: 25 - DNA, 20 bp, Artificial sequence
  primer
taagactctgcaggggtcca

SEQ ID NO: 26 - DNA, 20 bp, Artificial sequence
  primer
ccgagagccaacaatccagt